(19) United States
(12) Patent Application Publication
(10) Pub. No.: US 2019/0378997 A1
(43) Pub. Date: Dec. 12, 2019

(54) COMPOUND, LIGHT EMITTING MATERIAL AND ORGANIC LIGHT EMITTING ELEMENT

(71) Applicants: KYUSHU UNIVERSITY, NATIONAL UNIVERSITY CORPORATION, Fukuoka-shi, Fukuoka (JP); KING ABDULLAH UNIVERSITY OF SCIENCE AND TECHNOLOGY, Thuwal (SA)

(72) Inventors: Yuma ISHIKAWA, Fukuoka-shi, Fukuoka (JP); Youichi TSUCHIYA, Fukuoka-shi, Fukuoka (JP); Chihaya ADACHI, Fukuoka-shi, Fukuoka (JP); Jean-Luc BREDAS, Thuwal (SA); Xiankai CHEN, Thuwal (SA)

(21) Appl. No.: 16/489,385

(22) PCT Filed: Feb. 28, 2018
(86) PCT No.: PCT/JP2018/007461

§ 371 Date: Aug. 28, 2019

(30) Foreign Application Priority Data
Feb. 28, 2017 (JP) 2017-037588

(51) Int. Cl. H01L 51/00; C07D 471/16; C09K 11/06

(52) U.S. Cl. CPC H01L 51/0072; C07D 471/16; H01L 51/5012; H01L 51/0061; C09K 11/06

(57) ABSTRACT

A compound having a structure, represented by the following general formula is useful as a light emitting material. At least one of R¹ to R⁹ is a substituent; Y¹ to Y³ each are a methylene group, a carbonyl group, a thiocarbonyl group, an imino group, an oxygen atom, a sulfur atom or a sulfonyl group; Z is a nitrogen atom, a boron atom or a phosphine oxide group.

### TECHNICAL FIELD

The present invention relates to a compound, a light-emitting material containing the compound, and to an organic light-emitting device using the compound.

### BACKGROUND ART

Studies for enhancing the light emission efficiency of organic light-emitting devices such as organic electroluminescent devices (organic EL devices) are being made actively. In particular, various kinds of efforts have been made for increasing light emission efficiency by newly developing and combining an electron transfer material, a hole transfer material, a light-emitting material and others to constitute an organic electroluminescent device. Among them, there is known a study relating to an organic electroluminescent device that utilizes a thermal activation type delayed fluorescent material.

A thermal activation type delayed fluorescent material is a compound which, after having transited to an excited triplet state, undergoes reverse intersystem crossing from the excited triplet state to an excited singlet state through absorption of thermal energy, and emits fluorescence when returning back from the excited singlet state to a ground state thereof. Fluorescence through the route is observed later than fluorescence from the excited singlet state directly occurring not via reverse intersystem crossing (ordinary fluorescence), and is therefore referred to as delayed fluorescence. For example, in current excitation of a compound, the occurring probability of the excited singlet state to the excited triplet state is 25%/75%, and therefore improvement of light emission efficiency by the fluorescence alone from the directly occurring excited singlet state is limited. On the other hand, a thermal activation type delayed fluorescent material can effectively utilize also the energy in the excited triplet state occurring in a probability of 75% for fluorescent emission, and can be therefore expected to provide a higher light emission efficiency.

Typical thermal activation type delayed fluorescent materials heretofore known in the art include those having a structure of a donor site (D site) and an acceptor site (A site) bonding to each other (D-A type structure) (for example, NPLs 1 to 4). It is recognized that structuring twisting the donor site (D) and the acceptor site (A) to each other is important for realizing high luminescent efficiency.

### CITATION LIST

### Non-Patent Literature

### NPL 1: Nature. 2012, 492, 234

NPL 2: Chem. Commun. 2012, 48, 11392
NPL 3: Chem. Commun. 2013, 49, 10385
NPL 4: Nat. Photon. 2014, 8, 326

### SUMMARY OF INVENTION

### Technical Problem

On the other hand, regarding compounds not having structural twisting between a donor site (D) and an acceptor site (A) therein and compounds in which a donor site (D) and an acceptor site (A) do not bond to each other, studies for realizing excellent emission properties are not as yet sufficiently carried out, Given the situation, the present inventors have begun to take up molecular planning of light-emitting materials from a novel viewpoint and have made assiduous studies for the purpose of providing a novel light-emitting material.

### Solution to Problem

As a result of earnest investigations made for attaining the above-mentioned object, the inventors have completed a novel invention described below,

[1] A compound having a structure represented by the following general formula (1):

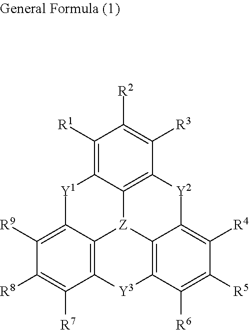

wherein R¹ to R⁹ each independently represent a hydrogen atom or a substituent, and at least one of R¹ to R⁹ is a substituent; Y¹ to Y³ each independently represent a substituted or unsubstituted methylene group (C(R¹⁰)(R¹¹) where R¹⁰ and R¹¹ each independently represent a hydrogen atom or a substituent), a carbonyl group (C═O), a thiocarbonyl group (C═S), a substituted or unsubstituted imino group ((R¹²) where R¹² represents a hydrogen atom or a substituent), an oxygen atom, a sulfur atom, or a. sulfonyl group (SO₂); Z represents a nitrogen atom, a boron atom, or a phosphine oxide group (P═O).
[2] The compound according to [1], wherein Z in the general formula (1) is a nitrogen atom.
[3] The compound according to [1] or [2], wherein Y¹ to Y³ in the general formula (1) each are a carbonyl group or a dialkylmethylene group.
[4] The compound according to any one of [1] to [3], wherein. R² in the general formula (1) is a substituent.
[5] The compound according to [4], wherein R² in the general formula (1) is a donor group.
[6] The compound according to [4], wherein R² in the general formula (1) is an acceptor group.
[7] The compound according to any one of [4] to [6], wherein a hydrogen bond can be formed between R¹ and R² in the general formula (1).
[8] The compound according to [7], wherein a hydrogen bond can be formed also between R² and R³.
[9] The compound according to any one of [1] to [8], having a line-symmetrical axis in the molecule.

[10] The compound according to any one of [1] to [9], represented by the following general formula (2):

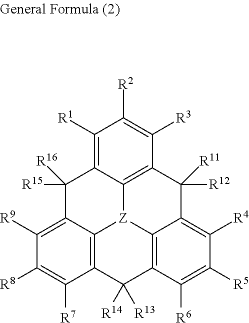

wherein R¹ to R⁹ each independently represent a hydrogen atom or a substituent, and at least one of R¹ to R⁹ is a substituent; R¹¹ to R¹⁶ each independently represent a substituent; Z represents a nitrogen atom, a boron atom, or a phosphine oxide group (P═O).
[11] The compound according to [10], wherein R² in the general formula (2) is a substituted or unsubstituted heteroaryl group.
[12] The compound according to [11], wherein the heteroaryl group contains a nitrogen atom as a ring skeleton-constituting atom.
[13] The compound according to [12], wherein at least one ring skeleton-constituting atom adjacent to the atom participating in bonding of the heteroaryl group is a nitrogen atom.
[14] The compound according to [13], wherein R¹ in the general formula (2) is a hydrogen atom.
[15] The compound according to [12], wherein all ring skeleton-constituting atoms adjacent to the atom participating in bonding of the heteroaryl group are nitrogen atoms.
[16] The compound according to [15], wherein the heteroaryl group is a substituted or unsubstituted triazinyl group.
[17] The compound according to [16], wherein the heteroaryl group is a substituted or unsubstituted diaryltriazinyl group.
[18] The compound according to any one of [15] to [17], wherein R¹ and R³ in the general formula (2) are hydrogen atoms.
[19] The compound according to any one of [1, ] to [18], wherein at least one of R⁴ to R⁶ and at least one of R⁷ to R⁹ in the general formula (2) each are a group containing a diarylamino structure or a carbazole ring.
[20] The compound according to [19], wherein R⁵ and R⁸ in the general formula (2) each are a group containing a diarylamino structure or a carbazole ring.
[21] The compound according to any one of [11] to [20], wherein at least one of R⁴ to R⁶ and at least one of R⁷ to R⁹ in the general formula (2) each are a group having a structure represented by the following general formula (4):

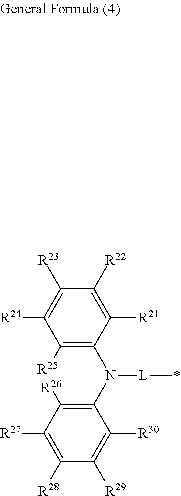

wherein R²¹ to R³⁰ each independently represent a hydrogen atom or a substituent; R²⁵ and R²⁶ may bond to each other to form a single bond or a linking group; L represents a single bond or a substituted or unsubstituted arylene group; * represents a bonding position.
[22] The compound according to [21], wherein R²⁵ and R²⁶ in the general formula (4) do not bond to each other.
[23] The compound according to [21] or [22], wherein at least one of R²³ and R²⁵ in the general formula (4) is a substituent.
[24] The compound according to any one of [21] to [23], wherein L in the general formula (4) is a single bond.
[25] The compound according to any one of [10] to [24], wherein R¹¹ to R¹⁶ in the general formula (2) each are independently a substituted or unsubstituted alkyl group.
[26] The compound according to [25], wherein R¹¹ to R¹⁶ in the general formula (2) are methyl groups.
[27] The compound according to any one of [1] to [9], represented by the following general formula (3):

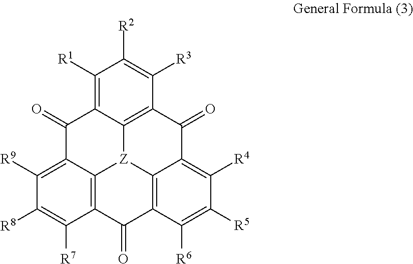

wherein R¹ to R⁹ each independently represent a hydrogen atom or a substituent, at least one of R¹ to R⁹ is a substituent; Z represents a nitrogen atom, a boron atom or a phosphine oxide group (P═O).
[28] The compound according to [27], wherein at least one of R¹ to R⁹ in the general formula (3) is a donor group.
[29] The compound according to [27] or [28], wherein R² in the general formula (3) is a group containing a diarylamino structure or a carbazole ring.
[30] The compound according to any one of [27] to [29], wherein at least one of R¹ to R⁹ in the general formula (3) is a group having a structure represented by the following general formula (4):

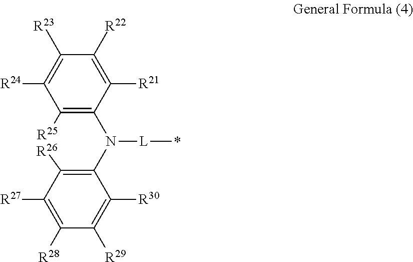

wherein R²¹ to R³⁰ each independently represent a hydrogen atom or a substituent; R²⁵ and R²⁶ may bond to each other to form a single bond or a linking group; L represents a single bond or a substituted or unsubstituted arylene group; * represents a bonding position.
[31] The compound according to [30], wherein R²⁵ and R²⁶ in the general formula (4) do not bond to each other.
[32] The compound according to [30], wherein R²⁵ and R²⁶ in the general formula (4) bond to each other to form a single bond.
[33] The compound according to any one of [30] to [32], wherein L in the general formula (4) is a substituted or unsubstituted phenylene group.
[34] The compound according to any one of [30] to [32], wherein L in the general formula (4) is a single bond.
[35] The compound according to any one of [30] to [34], wherein R² in the general formula (3) is a group represented by the general formula (4).
[36] The compound according to any one of [1] to [35], which emits delayed fluorescence.
[37] A light-emitting material containing a compound having a structure represented by the general formula (1).
[38] An organic light-emitting device containing a compound having a structure represented by the general formula. (1).
[39] The organic light-emitting device according to [38], wherein tile device is an organic electroluminescent device.
[40] The organic light-emitting device according to [38] or [39], which emits delayed fluorescence.

### BRIEF DESCRIPTION OF DRAWING

FIG. 1 is a schematic cross sectional view showing an example of a layer structure of an organic electroluminescent device.

### DESCRIPTION OF EMBODIMENTS

The contents of the invention will be described in detail below. The constitutional elements may be described below with reference to representative embodiments and specific examples of the invention, but the invention is not limited to the embodiments and the examples. In the description herein, a numerical range expressed as “to” means a range that includes the upper limit and/or the lower limit. In the invention, the hydrogen atom that is present in the compound used in the invention is not particularly limited in isotope species, and for example, all the hydrogen atoms in the molecule may be ¹H, and all or a part of them may be ²H (deuterium. (D)).

The entire disclosure of Japanese Patent Application 2017-37588 is incorporated herein by reference as a part of the present application.

### Compound represented by General Formula (1)

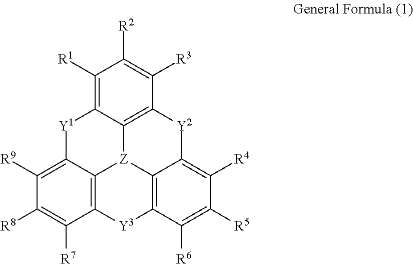

The compound having a structure represented by the general formula (1) of the present invention is described.

In the general formula (1), R¹ to R⁹ each independently represent a hydrogen atom or a substituent. Examples of the substituents that R¹ to R⁹ can take include a hydroxy group, a halogen atom, a cyano group, an alkyl group having 1 to 20 carbon atoms, an alkoxy group having 1 to 20 carbon atoms, an alkylthio group having 1 to 20 carbon atoms, an alkyl-substituted amino group having 1 to 20 carbon atoms, an aryl-substituted amino group having 12 to 40 carbon atoms, an acyl group having 2 to 20 carbon atoms, an aryl group having 6 to 40 carbon atoms, a heteroaryl group having 3 to 40 carbon atoms, a substituted or unsubstituted carbazolyl group having 12 to 40 carbon atoms, an alkenyl group having 2 to 10 carbon atoms, an alkynyl group having 2 to 10 carbon atoms, an alkoxycarbontyl group having 2 to 10 carbon atoms, an alkylsulfonyl group having 1 to 10 carbon atoms, a haloalkyl group having 1 to 10 carbon atoms, an amide group, an alkylamide group having 2 to 10 carbon atoms, a trialkylsilyl group having 3 to 20 carbon atoms, a trialkylsilylalkyl group having 4 to 20 carbon atoms, a trialkylsilylalkenyl group having 5 to 20 carbon atoms, a trialkylsilylalkynyl group having 5 to 20 carbon atoms, and a nitro group. Of such specific examples, those further substitutable with a substituent may be substituted. More preferred substituents include a halogen atom, a cyano group, a substituted or unsubstituted alkyl group having 1 to 20 carbon atoms, an alkoxy group having 1 to 20 carbon atoms, a substituted or unsubstituted aryl group having 6 to 40 carbon atoms, a substituted or unsubstituted heteroaryl group having 3 to 40 carbon atoms, a substituted or unsubstituted dialkylamino group having 1 to 10 carbon atoms, a substituted or unsubstituted diarylamino group having 12 to 40 carbon atoms, and a substituted or unsubstituted carhazolyl group having 12 to 40 carbon atoms. Even more preferred substituents include a fluorine atom, a chlorine atom, a cyano group, a substituted or unsubstituted alkyl group having 1 to 10 carbon atoms, a substituted or unsubstituted alkoxy group having 1 to 10 carbon atoms, a substituted or unsubstituted dialkylamino group having 1 to 10 carbon atoms, a substituted or unsubstituted diarylamino group having 12 to 40 carbon atoms, a substituted or unsubstituted aryl group having 6 to 15 carbon atoms, and a substituted or unsubstituted heteroaryl group having 3 to 12 carbon atoms,

At least one of R¹ to R⁹ in the general formula (1) is a substituent. Above all, preferably, one to three selected from R², R⁵ and R⁸ each are a substituent. Also preferably, at least one substituent is a donor group or an acceptor group.

In this description, “donor group” means a group having a Hammett's σₚ+value of less than 0. Examples of the “donor group” that may be employed in this description are those having a Hammett's σp+value of less than −0.15, those having the value of less than −0.3, those having the value of −0.45 or less, and those having the value of −0.6 or less. Examples of the “donor group” that may be employed in this description include those having a Hammett's σ+value of −2 or more and those having the value of −1 or more.

In this description, “acceptor group” means a group having a Hammett's σₚ+value of more than 0. Examples of the “acceptor group” employable in this description include those having a Hammett's σₚ+value of 0.15 or more, those having the value of 0.3 or more, those having the value of 0.45 or more, and those having the value of 0.6 or more. Examples of the “acceptor group” employable in this description include those having a Hammett's σₚ+value of 2 or less, and those having the value of 1 or less.

“Hammett's σₚ+value” In this description is one propounded by L. P. Hammett, and is one to quantify the influence of a substituent on the reaction rate or the equilibrium of a para-substituted benzene derivative. Specifically, the value is a constant (σₚ) peculiar to the substituent in the following equation that is established between a substituent and a reaction rate constant or an equilibrium constant in a para-substituted benzene derivative;

log(k/k₀)=ρσₚ or log(K/K₀)=ρσₚ

In the above equations, k represents a rate constant of a benzene derivative not having a substituent; k₀ represents a rate constant of a benzene derivative substituted with a substituent; K represents an equilibrium constant of a benzene derivative not having a substituent; K₀ represents an equilibrium constant of a benzene derivative substituted with a substituent; ρ represents a reaction constant to be determined by the kind and the condition of reaction. Regarding the description relating to the Hammett's σₚ value and the numerical value of each substituent, reference may be made to J. A. Dean's “Lange's Handbook of Chemistry, 13th Ed.”, 1985, pp. 3-132 to 3-137, McGraw-Hill.

In the general formula (1), Y¹ to Y³ each independently represent a substituted or unsubstituted methylene group (C(R¹⁰)(R¹¹) where R¹⁰ and R¹¹ each independently represent a hydrogen atom or a substituent), a carbonyl group (C═O), a thiocarbonyl group (C═S), a substituted or unsubstituted imino group (N(R¹²) where R¹² represents a hydrogen atom or a substituent), an oxygen atom, a sulfur atom, or a sulfonyl group (SO₂). Y¹ to Y³ may be the same or different, but preferably all Y¹ to Y³ are the same. Preferably, R¹⁰ and R¹¹ in the methylene group C(R¹⁰(R¹¹) which Y¹ to Y³ may take each independently represent a substituent, more preferably a substituted or unsubstituted alkyl group, even more preferably a substituted or unsubstituted alkyl group having 1 to 3 carbon atoms. Preferably, R¹² in the imino group N(R¹²) which Y¹ to Y³ may take each independently represent a substituent, more preferably a substituted or unsubstituted alkyl group, even more preferably a substituted or unsubstituted alkyl group having 1 to 3 carbon atoms.

Z in the general formula (1) represents a nitrogen atom, a boron atom, or a phosphine oxide group (P═O).

The compound represented by the general formula (1) may have a symmetrical structure. For example, a compound having a line-symmetrical axis is preferably employed. A compound having a rotationally symmetrical structure with the center atom Z as a central axis is also preferably employed.

The compound represented by the general formula (1) is also preferably one capable of forming a hydrogen bond between R¹ and R². The compound represented by the general formula (1) is also preferably one capable of forming a hydrogen bond between. R¹ and R² and additionally capable of forming a hydrogen bond between R² and R³. Also preferably, such a hydrogen bond can be formed between R⁴ and R⁵, between R⁵ and R⁶, between R⁷ and R⁸, and between R⁸ and R⁹. A hydrogen bond may be formed, for example, between a hydrogen atom and a nitrogen atom, and specifically, a case where a hydrogen bond is formed between a hydrogen atom and a nitrogen atom that is a ring skeleton-constituting atom of a heteroaryl group can be exemplified.

As the compound represented by the general formula (1), preferably employed is a compound represented by the general formula (2).

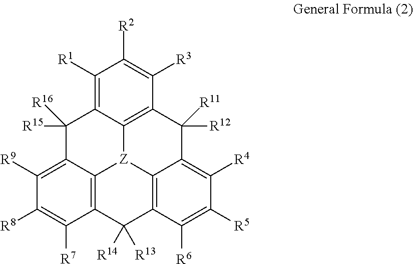

In the general formula (2), R¹ to R⁹ each independently represent a hydrogen atom or a substituent, and at least one of R¹ to R⁹ is a substituent. R¹¹ to R¹⁶ each independently represent a substituent. Z represents a nitrogen atom, a boron atom, or a phosphine oxide group (P═O). For the description, the preferred ranges and the specific examples of the substituent that R¹ to R⁹ in the general formula (2) may take, reference may be made to the corresponding description relating to the general formula (1).

R² in the general formula (2) is preferably a substituted or unsubstituted heteroaryl group, more preferably one containing a nitrogen atom as a ring skeleton-constituting atom, and even more preferably one in which both the ring skeleton-constituting atoms adjacent to the atom participating to bonding to the heteroaryl group are nitrogen atoms. A specific example of the heteroaryl group is a substituted or unsubstituted triazinyl group, and a preferred example thereof is a substituted or unsubstituted diaryltriazinyl group. When both the ring skeleton-constituting atoms adjacent to the atom participating to bonding to the heteroaryl group are nitrogen atoms and when R¹ or R³ is a hydrogen atom, a hydrogen bond can be formed.

When R² in the general formula (2) is a substituted or unsubstituted heteroaryl group, all R¹, and R³ to R⁹ may be hydrogen atoms, or at least one of them may be a substituent, but preferably, at least one of R⁴ to R⁶, and R⁷ to R⁹ is a group containing a diarylamino structure or a carbazole ring, more preferably at least one of R⁴ to R⁶, and at least one of R⁷ to R⁹ each are a group containing a diarylamino structure or a carbazole ring, and even more preferably R⁵ and R⁸ each are a group containing a diarylamino structure or a carbazole ring. For the description and the preferred range of the group containing a diarylamino structure or a carbazole ring, reference may be made to the description and the preferred ranges of the group containing a diarylamino structure and the group containing a carbazole ring that R¹ to R⁹ in the general formula (3) may take. When the group containing a diarylamino structure in the general formula (2) is a group represented by the general formula (4), preferably, R²⁵ and R²⁶ do not bond to each other, also preferably, at least one of R²³ and R²⁸ is a substituent, also preferably L is a single bond, and more preferably, these preferred configurations are all combined. The substituent in R²³ and R²⁸ is preferably a substituted or unsubstituted alkoxy group having 1 to 10 carbon atoms.

Preferably, R¹¹ to R¹⁶ in the general formula (2) each are independently a substituted or unsubstituted alkyl group, more preferably a substituted or unsubstituted alkyl group having 1 to 6 carbon atoms, even more preferably a substituted or unsubstituted alkyl group having 1 to 3 carbon atoms, For example, there are mentioned a methyl group and an ethyl group.

As the compound represented by the general formula (1), also preferably employable is a compound represented by the general formula (3).

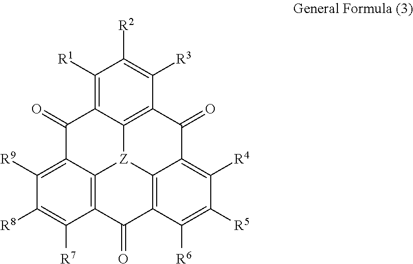

In the general formula (3), R¹ to R⁹ each independently represent a hydrogen atom or a substituent, at least one of R¹ to R⁹ is a substituent, Z represents a nitrogen atom, a boron atom or a phosphine oxide group (P═O). For the description and the preferred ranges of the substituents that R¹ to R⁹ may take, reference may be made to the corresponding description relating to the general formula (1). At least one of R¹ to R⁹ in the general formula (3) is preferably a donor group.

At least one of R¹ to R⁹ in the general formula (3) is preferably a group containing a diarylamino structure or a group containing a carbazole ring, and more preferably, R² is a group containing a diarylamino structure or a group containing a carbazole ring. Here, “diarylamino structure” means both a diarylamino group and a heteroaromatic ring structure of a diarylamino group where both the aryl groups bond to each other via a single bond or a linking group to form a hetero ring. The aromatic ring constituting each aryl group of the diarylamino structure may be a monocycle, or a condensed ring formed of 2 or more aromatic rings condensing together, or a linked ring formed of 2 or more aromatic rings linking to each other. In the case where 2 or more aromatic rings link to each other, they may link linearly or may link in a branched state, The carbon number of the aromatic ring constituting each aryl group of the diarylamino structure is preferably 6 to 22, more preferably 6 to 18, even more preferably 6 to 14, still more preferably 6 to 10. Specific examples of the aryl group include a phenyl group, a naphthyl group, and a biphenyl group; and a phenyl group is preferred. For the description and the preferred range of the diarylamino structure having a substituent, reference may be made to the description and the preferred ranges of the substituents that R²¹ to R³⁰ in the general formula (4) may take. For the description and the preferred range of the linking group that links aryl groups in the case where the diarylamino structure is the above-mentioned heteroaromatic ring structure, reference may be made to the description and the preferred range of the linking group in the case where R²⁵ and R²⁶ in the following general formula (4) link to each other to form a linking group. Of the group containing a diarylamino structure, the case where each aryl group of the diarylamino structure is a phenyl group and the phenyl groups link to each other via a single bond, the diarylamino structure-containing group corresponds to the above-mentioned carbazol ring-containing group.

In the group containing a diarylamino structure, the nitrogen atom to which each aryl group of the diarylamino structure bonds may bond to the benzene ring in the general formula (3) via a single bond or may link thereto via a linking group. Specifically, the group containing a diarylamino structure may contain a linking group that links the diarylamino structure to the benzene ring. The linking group that links the diarylamino structure to the benzene ring is, though not specifically limited thereto, preferably a substituted or unsubstituted arylene group. For the description, the preferred range and the specific examples of the substituted or unsubstituted arylene group, reference may be made to the description, the preferred range and the specific examples of the substituted or unsubstituted arylene group of L in the following general formula (4).

Especially, the group containing a diarylamino group is preferably a group having a structure represented by the general formula (4).

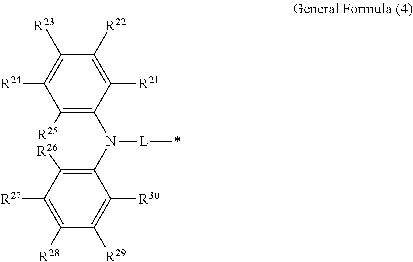

In the general formula (4), R²¹ to R³⁰ each independently represent a hydrogen atom or a substituent. The number of the substituents is not specifically limited, and all R²¹ to R³⁰ may be unsubstituted (that is, hydrogen atoms). In the case where 2 or more of R²¹ to R³⁰ are substituents, the plural substituents may be the same as or different from each other.

Examples of the substituent that R²¹ to R³⁰ may take include a hydroxy group, a halogen atom, an alkyl group having 1 to 20 carbon atoms, an alkoxy group having 1 to 20 carbon atoms, an alkylthio group having 1 to 20 carbon atoms, an alkyl-substituted amino group having 1 to 20 carbon atoms, an aryl-substituted amino group having 12 to 40 carbon atoms, an aryl group having 6 to 40 carbon atoms, a heteroaryl group having 3 to 40 carbon atoms, an alkenyl group having 2 to 10 carbon atoms, an alkynyl group having 2 to 10 carbon atoms, an alkylamide group having 2 to 20 carbon atoms, an arylamide group having 7 to 21 carbon atoms, and a trialkylsilyl group having 3 to 20 carbon atoms. Of these substituents, those further substitutable with any substituent may be substituted. More preferred substituents are an alkyl group having 1 to 20 carbon atoms, an alkoxy group having 1 to 20 carbon atoms, an alkylthio group having 1 to 20 carbon atoms, an alkyl-substituted amino group having 1 to 20 carbon atoms, an aryl-substituted amino group having 12 to 40 carbon atoms, an aryl group having 6 to 40 carbon atoms, and a heteroaryl group having 3 to 40 carbon atoms.

R²⁵ and R²⁶ may bond to each other to form a single bond or a linking group. Among the groups represented by the general formula (4), preferred are those where R²⁵ and R²⁶ do not bond, those where R²⁵ and R²⁶ bond to each other to form a single bond, or those where R²⁵ and R²⁶ bond to each other to form a linking group in which the linking chain length is one atom; and more preferred are those where R²⁵ and R²⁶ do not bond, or those where R²⁵ and R²⁶ bond to each other to form a single bond. In the case where R²⁵ and R²⁶ bond to each other to form a linking group in which the linking chain length is one atom, the cyclic structure to be formed as a result of bonding of R²⁵ and R²⁶ to each other is a 6-membered ring. Specific examples of the linking group to be formed by R²⁵ and R²⁶ bonding to each other include linking groups represented by —O—, —S—, —N(R⁹¹)— of —C(R⁹²)(R⁹³)—. In these, R⁹¹ to R⁹³ each independently represent a hydrogen atom or a substituent. Examples of the substituent that R⁹¹ may take include an alkyl group having 1 to 20 carbon atoms, an aryl group having 6 to 40 carbon atoms, and a heteroaryl group having 3 to 40 carbon atoms. Examples of the substituent that R⁹² and R⁹³ may take each independently represent a hydroxy group, a halogen atom, an alkyl group having 1 to 20 carbon atoms, an alkoxy group having 1 to 20 carbo atoms, an alkylthio group having 1 to 20 carbon atoms, an alkyl-substituted amino group having 1 to 20 carbon atoms, an aryl-substituted amino group having 12 to 40 carbon atoms, an aryl group having 6 to 40 carbon atoms, a heteroaryl group having 3 to 40 carbon atoms, an alkenyl group having 2 to 10 carbon atoms, an alkynyl group having 2 to 10 carbon atoms, an alkylamide group having 2 to 20 carbon atoms, an arylamide group having 7 to 21 carbon atoms, and a trialkylsilyl group having 3 to 20 carbon atoms.

L represents a single bond or a substituted or unsubstituted arylene group, and * represents a bonding position. For the description and the preferred range of the aromatic ring constituting the arylene group of L, reference may be made to the description and the preferred range of the aromatic ring constituting each aryl of the above-mentioned diarylamino structure; and for the description, the preferred range and the specific examples of the substituent in the case where the arylene group has a substituent, reference may be made to the description and the preferred range of the substituent that R²¹ to R³⁰ may take as mentioned above. Specific examples of the substituted or unsubstituted arylene group of L include a substituted or unsubstituted phenylene group, a substituted or unsubstituted naphthyiene group, and a substituted or unsubstituted biphenyl-diyl group; and a substituted or unsubstituted phenylene group is preferred.

In the following, specific examples of the compound represented by the general formula (1) are exemplified. However, the compound represented by the general formula (1) for use in the present invention should not be limitatively interpreted by these specific examples.

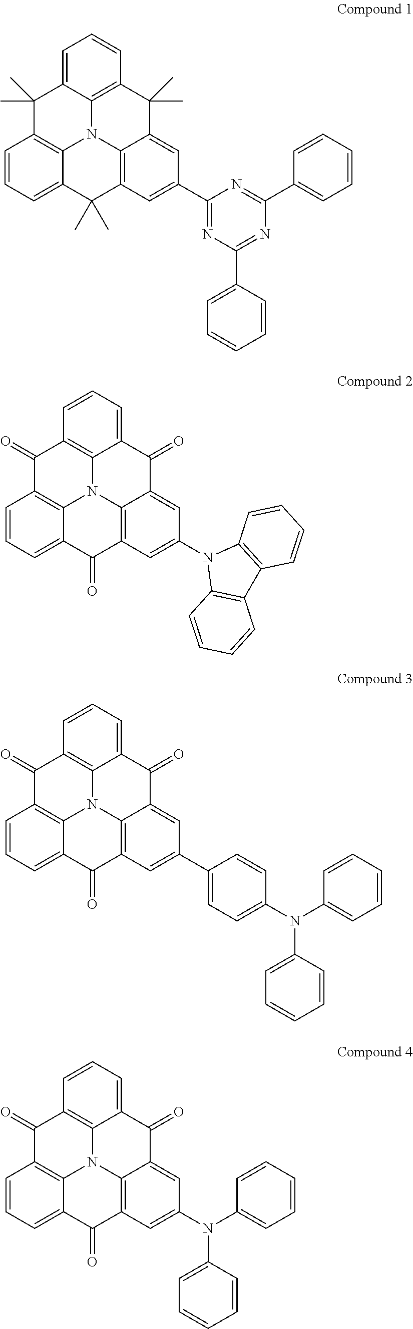

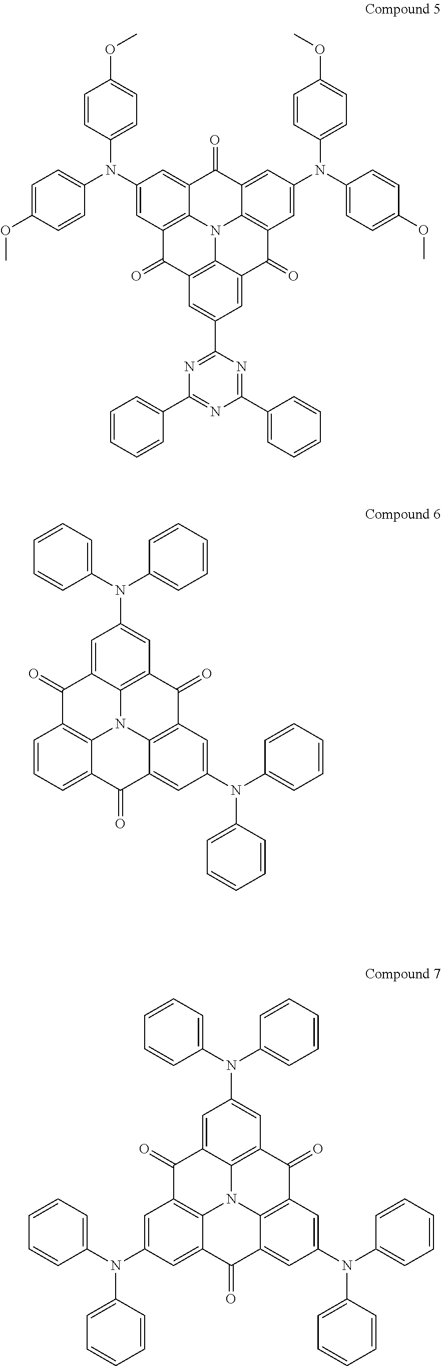

The compound represented by the general formula (1) is preferably such that the difference ΔE_{ST} between the lowest excited singlet energy level E_{S1} and the lowest excited triplet energy level E_{T1} thereof in an m-CBP-doped state is 0.4 eV or less, more preferably 0.2 eV or less, even more preferably 0.1 eV or less. For the method of measuring ΔE_{ST}, reference may be made to the section of Examples.

The compound represented by the general formula (1) can emit delayed fluorescence. Accordingly, the present invention includes an invention of a delayed fluorescent material having a structure represented by the general formula (1).

The molecular weight of the compound represented by the general formula (1) is, for example, in the case where an organic layer containing the compound represented by the general formula (1) is intended to be formed according to a vapor deposition method and used in devices, preferably 1500 or less, more preferably 1200 or less, even more preferably 1000 or less, and further more preferably 800 or less. The lower limit of the molecular weight is the smallest molecular weight that the general formula (1) can take.

Irrespective of the molecular weight thereof, the compound represented by the general formula (1) may be formed into a film according to a coating method. When a coating method is employed, even a compound having a relatively large molecular weight can be formed into a film.

The compound capable of emitting delayed fluorescence and capable of undergoing intramolecular proton transfer may be a polymer produced through polymerization of a polymerizable monomer capable of emitting delayed fluorescence and capable of undergoing intramolecular proton transfer.

For example, it is considered that a polymerizable group is previously introduced into a structure represented by the general formula (1) and the polymerizable group is polymerized to give a polymer, and the polymer is used as a light-emitting material. Specifically, a monomer containing a polymerizable functional group in any of R¹ to R⁹ or Y¹ to Y³ in the general formula (1) is prepared, and this is homo-polymerized or copolymerized with any other monomer to give a polymer having a recurring unit, and the polymer can be used as a material for organic light-emitting devices. Alternatively, compounds each having a structure represented by the general formula (1) are coupled to give a dimer or a trimer, and it can be used as a material for organic light-emitting devices.

Examples of the polymer having a recurring unit containing a structure represented by the general formula (1) include polymers containing a structure represented by the following general formula (11) or (12).

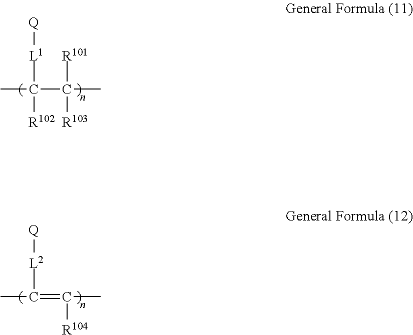

In the general formula (11) or (12), Q represents a group containing a structure represented by the general formula (1), and L¹ and L² each represent a linking group. The carbon number of the linking group is preferably 0 to 20, more preferably 1 to 15, even more preferably 2 to 10. Preferably, the linking group has a structure represented by —X¹¹-L¹¹-. Here, X¹¹ represents an oxygen atom or a sulfur atom and is preferably an oxygen atom. L¹¹ represents a linking group, and is preferably a substituted or unsubstituted alkylene group, or a substituted or unsubstituted arylene group, more preferably a substituted or unsubstituted alkylene group having 1 to 10 carbon atoms, or a substituted or unsubstituted phenylene group.

In the general formula (11) or (12), R¹⁰¹ , R¹⁰², R¹⁰³ and R¹⁰⁴ each independently represent a substituent. Preferably, the substituent is a substituted or unsubstituted alkyl group having 1 to 6 carbon atoms, a substituted or unsubstituted alkoxy group having 1 to 6 carbon atoms, or a halogen atom, more preferably an unsubstituted alkyl group having 1 to 3 carbon atoms, an unsubstituted alkoxy group having 1 to 3 carbon atoms, a fluorine atom, or a chlorine atom, and even more preferably an unsubstituted alkyl group having 1 to 3 carbon atoms, or an unsubstituted alkoxy group having 1 to 3 carbon atoms.

The linking group represented by L¹ and L² may bond to any of R¹ to R⁹ or Y¹ to Y³ in the structure of the general formula (1) constituting Q. Two or more linking groups may bond to one Q to form a crosslinked structure or a network structure,

Specific examples of the structure of the repeating unit include structures represented by the following formulae (13) to (16).

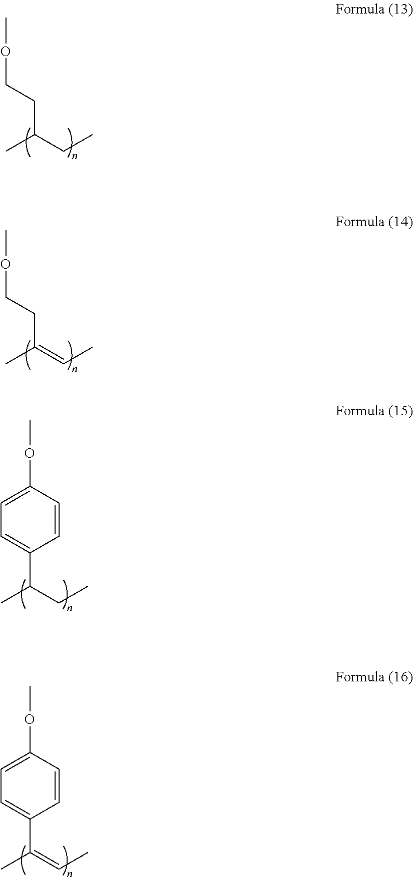

The polymer having the repeating unit containing the structure represented by any of the formulae (13) to (16) may be synthesized in such a manner that a hydroxyl group is introduced to any of R¹ to R⁹ or Y¹ to Y³ in the structure represented by the general formula (1), and the hydroxyl group as a linker is reacted with the following compound to introduce a polymerizable group thereto, followed by polymerizing the polymerizable group.

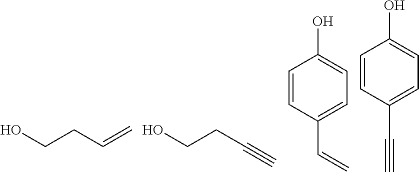

The polymer containing the structure represented by the general formula (1) in the molecule may be a polymer containing only a repeating unit having the structure represented by the general formula (1), or a polymer further containing a repeating unit having another structure. The repeating unit having the structure represented by the general formula (1) contained in the polymer may be only one kind or two or more kinds. Examples of the repeating unit that does not have the structure represented by the general formula (1) include a repeating unit derived from a monomer that is used for ordinary copolymerization. Examples of the repeating unit include a repeating unit derived from a monomer having an ethylenic unsaturated bond, such as ethylene and styrene.

### Synthesis Method for Compound Represented by General Formula (1)

A method for synthesizing the compound represented by the general formula (1) is not specifically limited. For example, for synthesis of the compound represented by the general formula (1) where R² is a substituent, a compound of the general formula (1) where R² is a hydrogen atom and a compound represented by R²—X are reacted, or a compound of the general formula (1) where R is a halogen atom and a compound represented by R²—H are reacted. Here, X is a halogen atom. The halogen atom is preferably a chlorine atom, a bromine atom or an iodine atom. For the details of the reaction, reference may be made to Synthesis Examples described later. The compound represented by the general formula (1) may be synthesized by combining the other known synthesis reactions.

### Organic Light-Emitting Device

The organic light-emitting material of the present invention uses a compound capable of emitting delayed fluorescence and capable of undergoing intramolecular proton transfer. The compound capable of emitting delayed fluorescence and capable of undergoing intramolecular proton transfer exhibits a sufficiently high quantum yield enough for practical use and can be effectively used as a light-emitting material in the organic light-emitting device. In addition, the compound capable of emitting delayed fluorescence and capable of undergoing intramolecular proton transfer can also be used as a host or an assist dopant for the organic light emitting device. For example, the organic light-emitting device using a compound capable of emitting delayed fluorescence and capable of undergoing intramolecular proton transfer as a light-emitting material is characterized by having a high emission efficiency since the compound therein functions as a delayed fluorescent material. The principle will be described below with reference to an organic electroluminescent device taken as an example.

In an organic electroluminescent device, carriers are injected from an anode and a cathode to a light-emitting material to form an excited state for the light-emitting material, with which light is emitted. In the case of a carrier injection type organic electroluminescent device, in general, excitons that are excited to the excited singlet state are 25% of the total excitons generated, and the remaining 75% thereof are excited to the excited triplet state. Accordingly, the use of phosphorescence, which is light emission from the excited triplet state, provides a high energy utilization. However, the excited triplet state has a long lifetime and thus causes saturation of the excited state and deactivation of energy through mutual action with the excitons in the excited triplet state, and therefore the quantum yield of phosphorescence may generally be often not high. A delayed fluorescent material emits fluorescent light through the mechanism that the energy of excitons transits to the excited triplet state through intersystem crossing or the like, and then transits to the excited singlet state through reverse intersystem crossing due to triplet-triplet annihilation or absorption of thermal energy, thereby emitting fluorescent light, It is considered that among the materials, a thermal activation type delayed fluorescent material emitting light through absorption of thermal energy is particularly useful for an organic electroluminescent device. In the case where a delayed fluorescent material is used in an organic electroluminescent device, the excitons in the excited singlet state normally emit fluorescent light. On the other hand, the excitons in the excited triplet state emit fluorescent light through intersystem crossing to the excited singlet state by absorbing outdoor air or the heat generated by the device. At this time, the light emitted through reverse intersystem crossing from the excited triplet state to the excited singlet state has the same wavelength as fluorescent light since it is light emission from the excited singlet state, but has a longer lifetime (light emission lifetime) than the normal fluorescent light, and thus the light is observed as fluorescent light that is delayed from the normal fluorescent light. The light may be defined as delayed fluorescent light. The use of the thermal activation type exciton transition mechanism may raise the proportion of the compound in the excited singlet state, which is generally formed in a proportion only of 25%, to 25% or more through the absorption of the thermal energy after the carrier injection. A compound that emits strong fluorescent light and delayed fluorescent light at a low temperature of lower than 100° C. undergoes the intersystem crossing from the excited triplet state to the excited singlet state sufficiently with the heat of the device, thereby emitting delayed fluorescent light, and thus the use of the compound may drastically enhance the light emission efficiency.

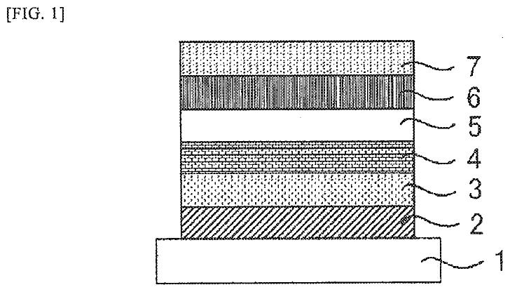

Using a compound capable of emitting delayed fluorescence and capable of undergoing intramolecular proton transfer as a light-emitting material in a light-emitting layer, excellent organic light-emitting devices such as organic photoluminescent device (organic PL device) and an organic electroluminescent device (organic EL device) can be provided. An organic photoluminescent device has a structure where at least a light-emitting layer is formed on a substrate. An organic electroluminescent device has a structure including at least an anode, a cathode and an organic layer formed between the anode and the cathode. The organic layer contains at least a light-emitting layer, and may be formed of a light-emitting layer alone, or may has one or more other organic layers in addition to a light-emitting layer. The other organic layers include a hole transport layer, a hole injection layer, an electron blocking layer, a hole blocking layer, an electron injection layer, an electron transport layer, and an exciton blocking layer. The hole transport layer may be a hole injection transport layer having a hole injection function, and the electron transport layer may be an electron injection transport layer having an electron injection function. A configuration example of an organic electroluminescent device is shown in FIG. 1. In FIG. 1, 1 is a substrate, 2 is an anode, 3 is a hole injection layer, 4 is a hole transport layer, 5 is a light-emitting layer, 6 is an. electron transport layer, and 7 is a cathode.

In the following, the constituent members and the layers of the organic electroluminescent device are described. The description of the substrate and the light-emitting layer given below may apply to the substrate and the light-emitting layer of an organic photoluminescent device.

### (Substrate)

The organic electroluminescent device of the invention is preferably supported by a substrate. The substrate is not particularly limited and may be those that have been commonly used in an organic electroluminescent device, and examples thereof used include those formed of glass, transparent plastics, quartz and silicon,

### (Anode)

The anode of the organic electroluminescent device used is preferably formed of, as an electrode material, a metal, an alloy, or an electroconductive compound each having a large work function (4 eV or more), or a mixture thereof. Specific examples of the electrode material include a metal, such as Au, and an electroconductive transparent material, such as CuI, indium tin oxide (ITO), SnO₂ and ZnO. A material that is amorphous and is capable of forming a transparent electroconductive film, such as IDIXO (In₂O₃—ZnO), may also be used. The anode may be formed in such a manner that the electrode material is formed into a thin film by such a method as vapor deposition or sputtering, and the film is patterned into a desired pattern by a photolithography method, or in the case where the pattern may not require high accuracy (for example, approximately 100 μm or more), the pattern may be formed with a mask having a desired shape on vapor deposition or sputtering of the electrode material. In alternative, in the case where a material capable of being coated, such as an organic electroconductive compound, is used, a wet film forming method, such as a printing method and a coating method, may be used. In the case where emitted light is to be taken out through the anode, the anode preferably has a transmittance of more than 10%, and the anode preferably has a sheet resistance of several hundred ohm per square or less. The thickness of the anode may be generally selected from a range of from 10 to 1,000 nm, and preferably from 10 to 200 nm, while depending on the material used.

### (Cathode)

The cathode is preferably formed of as an electrode material a metal (which is referred to as an electron injection metal), an alloy, or an electroconductive compound, having a small work function (4 eV or less), or a mixture thereof. Specific examples of the electrode material include sodium, a sodium-potassium alloy, magnesium, lithium, a magnesium-cupper mixture, a magnesium-silver mixture, a magnesium-aluminum mixture, a magnesium-indium mixture, an aluminum-aluminum oxide (Al₂O₃) mixture, indium, a lithium-aluminum mixture, and a rare earth metal. Among these, a mixture of an electron injection metal and a second metal that is a stable metal having a larger work function than the electron injection metal, for example, a magnesium-silver mixture, a magnesium-aluminum mixture, a magnesium-indium mixture, an aluminum-aluminum oxide (Al₂O₃) mixture, a lithium-aluminum mixture, and aluminum, is preferred from the standpoint of the electron injection property and the durability against oxidation and the like. The cathode may be produced by forming the electrode material into a thin film by such a method as vapor deposition or sputtering. The cathode preferably has a sheet resistance of several hundred ohm per square or less, and the thickness thereof may be generally selected from a range of from 10 nm to 5 μm, and preferably from 50 to 200 nm. For transmitting the emitted light, any one of the anode and the cathode of the organic electroluminescent device is preferably transparent or translucent, thereby enhancing the light emission luminance.

The cathode may be formed with the electroconductive transparent materials described for the anode, thereby forming a transparent or translucent cathode, and by applying the cathode, a device having an anode and a cathode, both of which have transmittance, may be produced.

### (Light-emitting Layer)

The light-emitting layer is a layer in which holes and electrons injected from an anode and a cathode are recombined to give excitons for light emission. A light-emitting material may be used singly in the light-emitting layer, but preferably, the layer contains a light-emitting layer and a host material. As the light-emitting material, one or more selected from a group of compounds capable of emitting delayed fluorescence and capable of undergoing intramolecular proton transfer can be used. In order that the organic electroluminescent device and the organic photoluminescent device of the present invention can express a high light emission efficiency, it is important to confine the singlet exciton and the triplet exciton formed in the light-emitting material to the light-emitting material. Accordingly, preferably, a host material is used in addition to the light-emitting material in the light-emitting layer. As the host material, an organic compound, of which at least any one of the excited singlet energy and the excited triplet energy is higher than that of the light-emitting material, may be used. As a result, the singlet exciton and the triplet exciton formed in the light-emitting material can be confined to the molecule of the light-emitting material to sufficiently derive the light emission efficiency thereof. Needless-to-say, there may be a case where a high light emission efficiency could be attained even though the singlet exciton and the triplet exciton could not be sufficiently confined, and therefore, any host material capable of realizing a high light emission efficiency can be used in the present invention with no specific limitation. In the organic light-emitting device or the organic electroluminescent device of the present invention, light emission occurs from the light-emitting material (compound capable of emitting delayed fluorescence and capable of undergoing intramolecular proton transfer) contained in the light-emitting layer. The light emission contains both of fluorescent emission and delayed fluorescent emission. In addition, a part of light emission may be partially from a host material.

In the case where a hots material is used, the content of the compound serving as a light-emitting material, that is, the compound capable of emitting delayed fluorescence and capable of undergoing intramolecular proton transfer in the light-emitting layer is preferably 0.1% by weight or more, more preferably 1% by weight or more, and is preferably 50% by weight or less, more preferably 20% by weigh tor less, even more preferably 10% by weight or less.

The host material in the light-emitting layer is preferably an organic compound having hole transport competence and electron transport competence, capable of preventing prolongation of emission wavelength and having a high glass transition temperature.

### (Injection Layer)

The injection layer is a layer that is provided between the electrode and the organic layer, for decreasing the driving voltage and enhancing the light emission luminance, and includes a hole injection layer and an electron injection layer, which may be provided between the anode and the light-emitting layer or the hole transport layer and between the cathode and the light emitting layer or the electron transport layer. The injection layer may be provided depending on necessity.

### (Blocking Layer)

The blocking layer is a layer that is capable of inhibiting charges (electrons or holes) and/or excitons present in the light-emitting layer from being diffused outside the light-emitting layer. The electron blocking layer may be disposed between the light-emitting layer and the hole transport layer, and inhibits electrons from passing through the light-emitting layer toward the hole transport layer. Similarly, the hole blocking layer may be disposed between the light-emitting layer and the electron transport layer, and inhibits holes from passing through the light-emitting layer toward the electron transport layer. The blocking layer may also be used for inhibiting excitons from being diffused outside the light-emitting layer. Thus, the electron blocking layer and the hole blocking layer each may also have a function as an exciton blocking layer. The term “the electron blocking layer” or “the exciton blocking layer” referred to herein is intended to include a layer that has both the functions of an electron blocking layer and an exciton blocking layer by one layer.

### (Hole Blocking Layer)

The hole blocking layer has the function of an electron transport layer in a broad sense. The hole blocking layer has a function of inhibiting holes from reaching the electron transport layer while transporting electrons, and thereby enhances the recombination probability of electrons and holes in the light-emitting layer. As the material for the hole blocking layer, the material for the electron transport layer to be mentioned below may be used optionally.

### (Electron Blocking Layer)

The electron blocking layer has the function of transporting holes in a broad sense. The electron blocking layer has a function of inhibiting electrons from reaching the hole transport layer while transporting holes, and thereby enhances the recombination probability of electrons and holes in the light-emitting layer.

### (Exciton Blocking Layer)

The exciton blocking layer is a layer for inhibiting excitons generated through recombination of holes and electrons in the light-emitting layer from being diffused to the charge transporting layer, and the use of the layer inserted enables effective confinement of excitons in the light-emitting layer, and thereby enhances the light emission efficiency of the device. The exciton blocking layer may be inserted adjacent to the light-emitting layer on any of the side of the anode and the side of the cathode, and on both the sides. Specifically, in the case where the exciton blocking layer is present on the side of the anode, the layer may be inserted between the hole transport layer and the light-emitting layer and adjacent to the light-emitting layer, and in the case where the layer is inserted on the side of the cathode, the layer may be inserted between the light-emitting layer and the cathode and adjacent to the light-emitting layer. Between the anode and the exciton blocking layer that is adjacent to the light-emitting layer on the side of the anode, a hole injection layer, an electron blocking layer and the like may be provided, and between the cathode and the exciton blocking layer that is adjacent to the light-emitting layer on the side of the cathode, an electron injection layer, an electron transport layer, a hole blocking layer and the like may be provided. In the case where the blocking layer is provided, preferably, at least one of the excited singlet energy and the excited triplet energy of the material used as the blocking layer is higher than the excited singlet energy and the excited triplet energy of the light-emitting layer, respectively, of the light-emitting material.

### (Hole Transport Layer)

The hole transport layer is formed of a hole transport material having a function of transporting holes, and the hole transport layer may be provided as a single layer or plural layers.

The hole transport material has one of injection or transporting property of holes and blocking property of electrons, and may be any of an organic material and an inorganic material. Examples of known hole transport materials that may be used herein include a triazole derivative, an oxadiazole derivative, an imidazole derivative, a carbazole derivative, an indolocarbazole derivative, a polyarylalkane derivative, a pyrazoline derivative, a pyrazolone derivative, a phenylenediamine derivative, an arylamine derivative, an amino-substituted chalcone derivative, an oxazole derivative, a styrylanthracene derivative, a fluorenone derivative, a hydrazone derivative, a stilbene derivative, a silazane derivative, an aniline copolymer and an electroconductive polymer oligomer, particularly a thiophene oligomer. Among these, a porphyrin compound, an aromatic tertiary amine compound and a styrylamine compound are preferably used, and an aromatic tertiary amine compound is more preferably used.

### (Electron Transport Layer)

The electron transport layer is formed of a material having a function of transporting electrons, and the electron transport layer may be a single layer or may be formed of plural layers.

The electron transport material (often also acting as a hole blocking material) may have a function of transmitting the electrons injected from a cathode to a light-emitting layer. The electron transport layer usable here includes, for example, nitro-substituted fluorene derivatives, diphenylquinone derivatives, thiopyran dioxide derivatives, carbodiimides, fluorenylidenemethane derivatives, anthraquinodimethane and anthrone derivatives, oxadiazole derivatives, etc. Further, thiadiazole derivatives derived from the above-mentioned oxadiazole derivatives by substituting the oxygen atom in the oxadiazole ring with a sulfur atom, and quinoxaline derivatives having a quinoxaline ring known as an electron-attractive group are also usable as the electron transport material. Further, polymer materials prepared by introducing these materials into the polymer chain, or having these material in the polymer main chain are also usable.

In producing the organic electroluminescent device, the compound capable of emitting delayed fluorescence and capable of undergoing intramolecular proton transfer may be used not only in the light-emitting layer but also in any other layer than the light-emitting layer. In so doing, the compound capable of emitting delayed fluorescence and capable of undergoing intramolecular proton transfer used in the light-emitting layer and the compound capable of emitting delayed fluorescence and capable of undergoing intramolecular proton transfer used in the other layer than the light-emitting layer may be the same as or different from each other. For example, the compound capable of emitting delayed fluorescence and capable of undergoing intramolecular proton transfer may be used in the above-mentioned injection layer, the blocking layer, the hole blocking layer, the electron blocking layer, the exciton blocking layer, the hole transport layer, and the electron transport layer. The method for forming these layers is not specifically limited, and the layers may be formed according to any of a dry process or a wet process.

Preferred materials for use for the organic electroluminescent device are concretely exemplified below. However, the materials for use in the present invention are not limitatively interpreted by the following exemplary compounds. Compounds, even though exemplified as materials having a specific function, can also be used as other materials having any other function.

First, preferred compounds for use as a host material in a light-emitting layer are mentioned below.

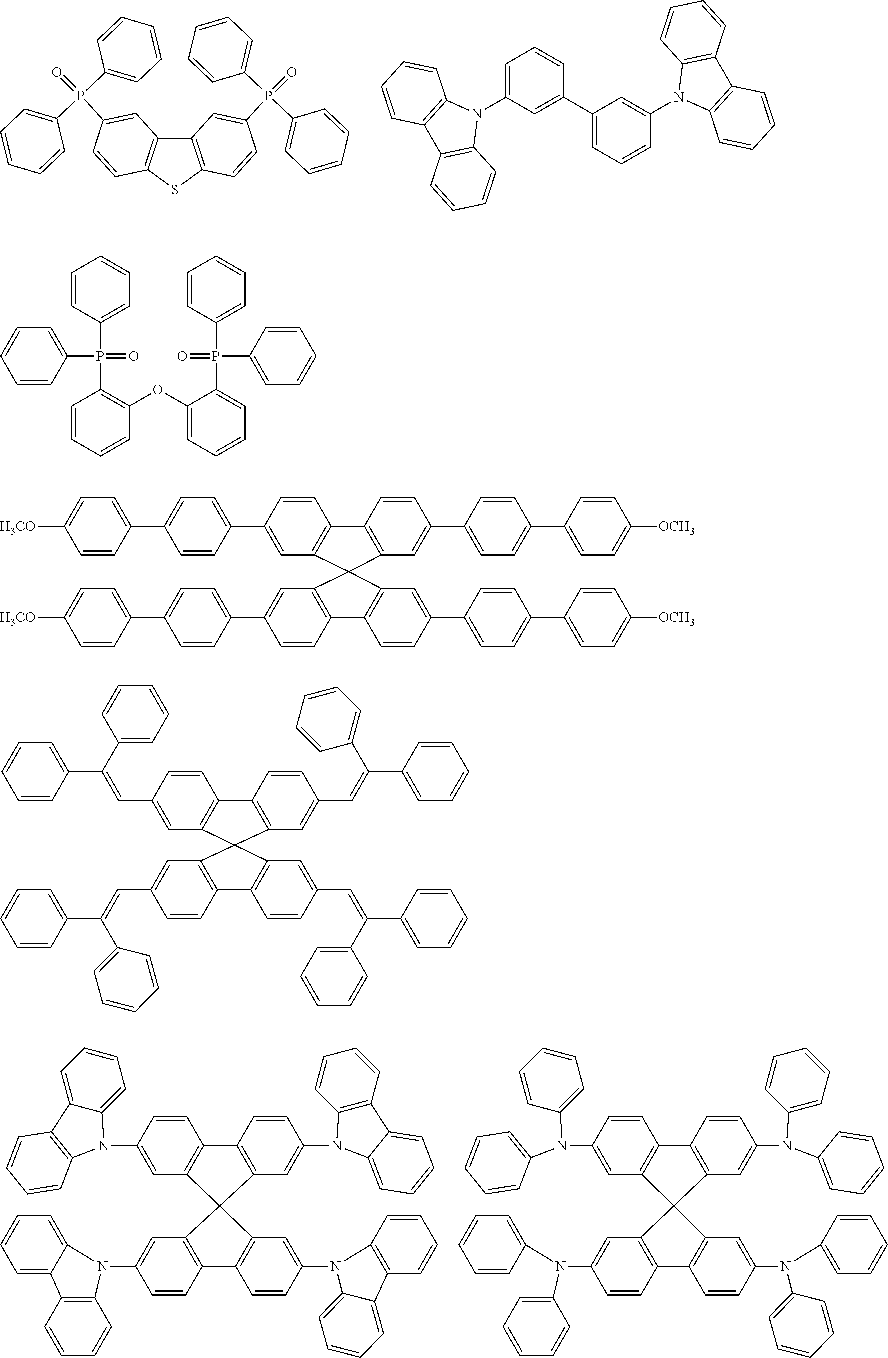

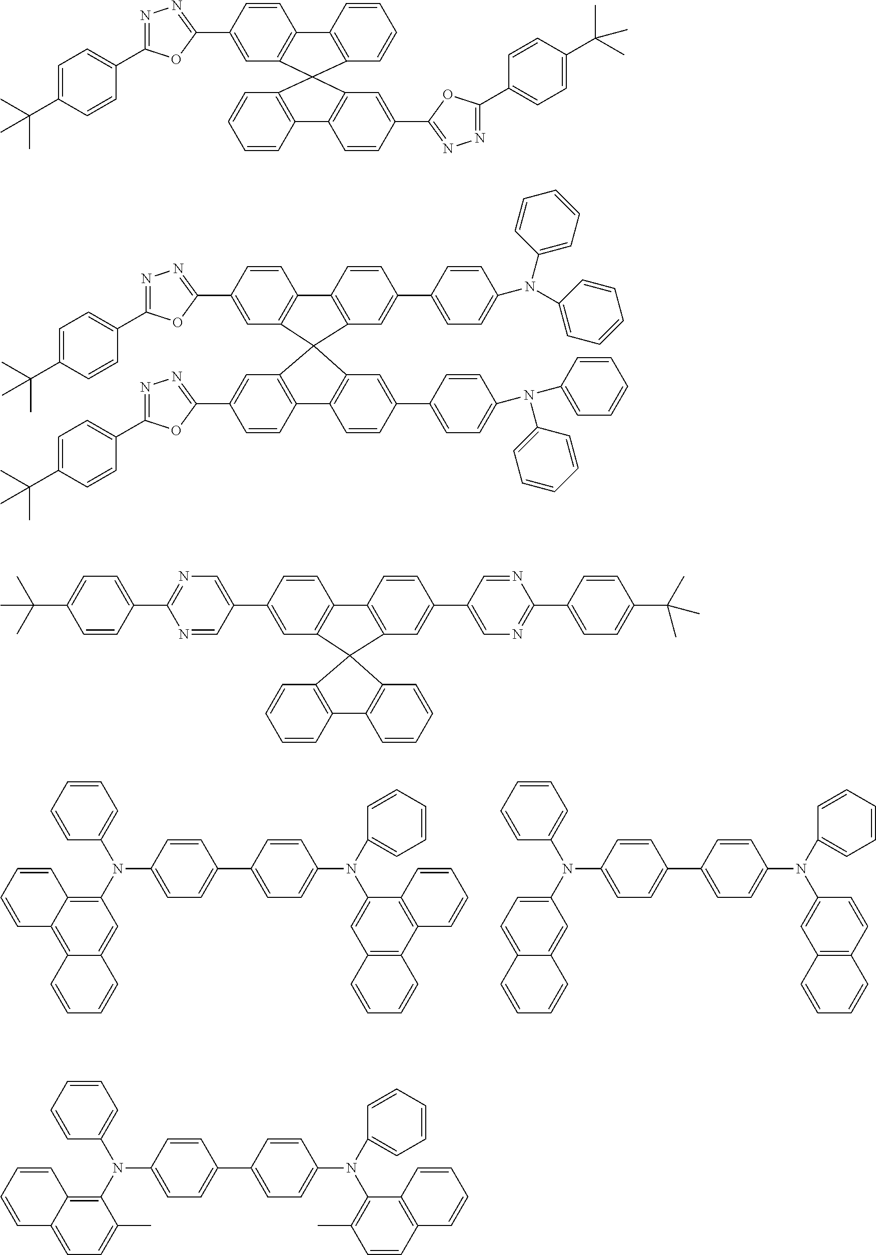

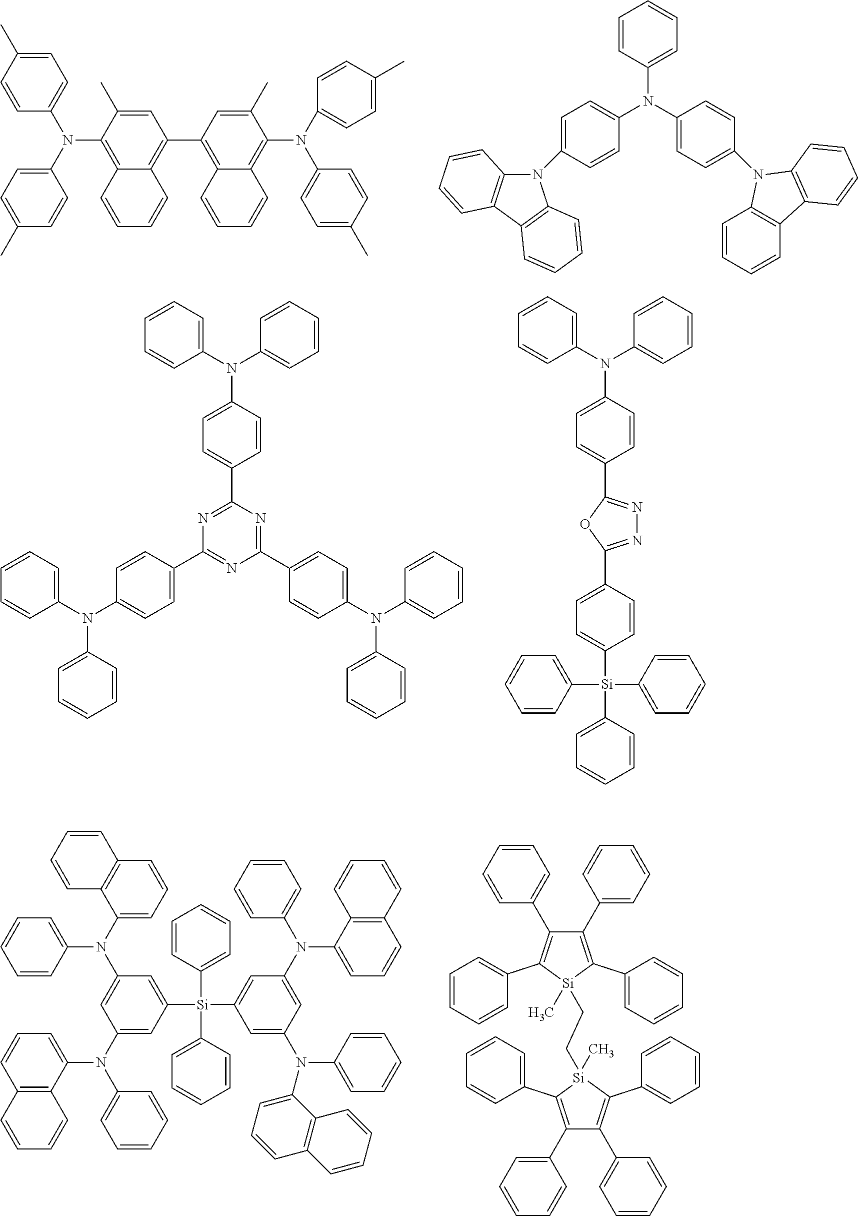

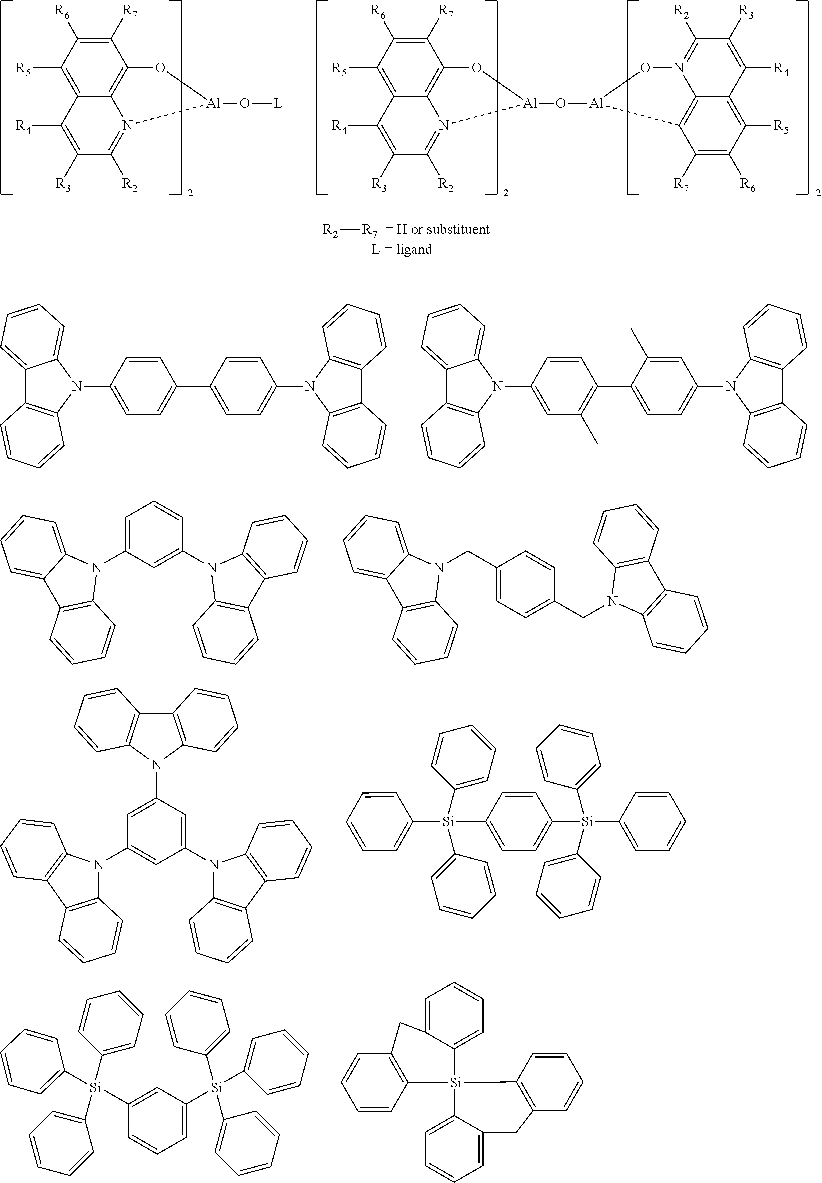

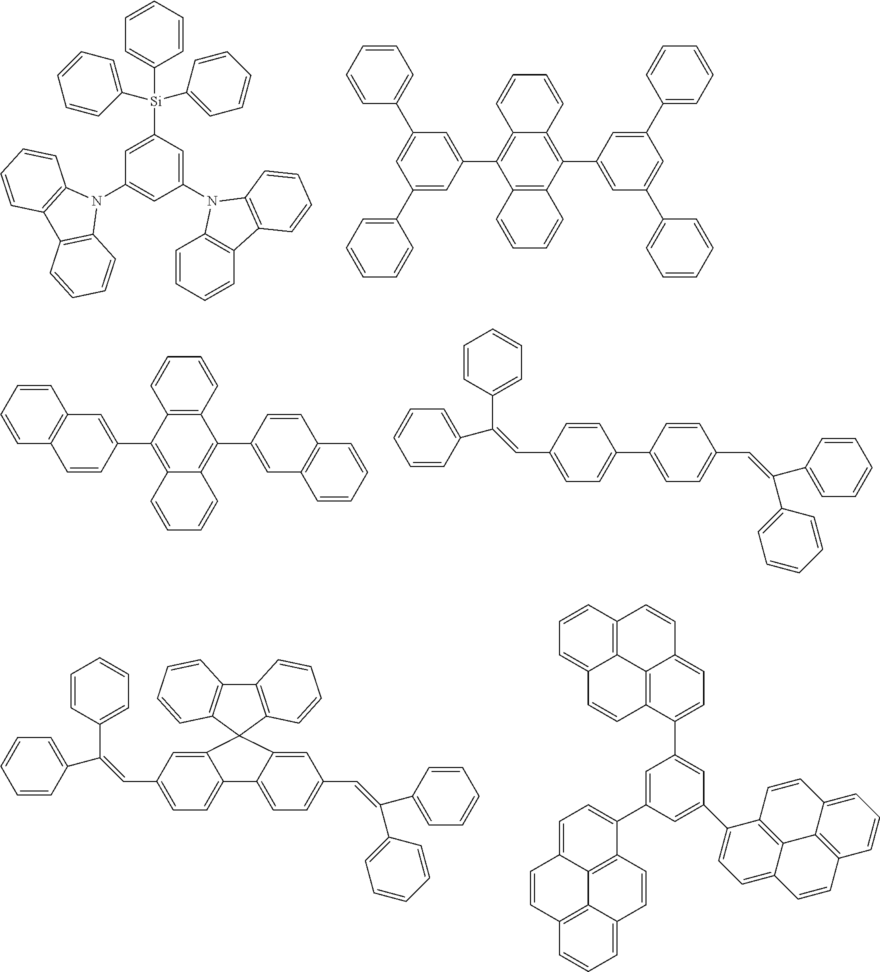

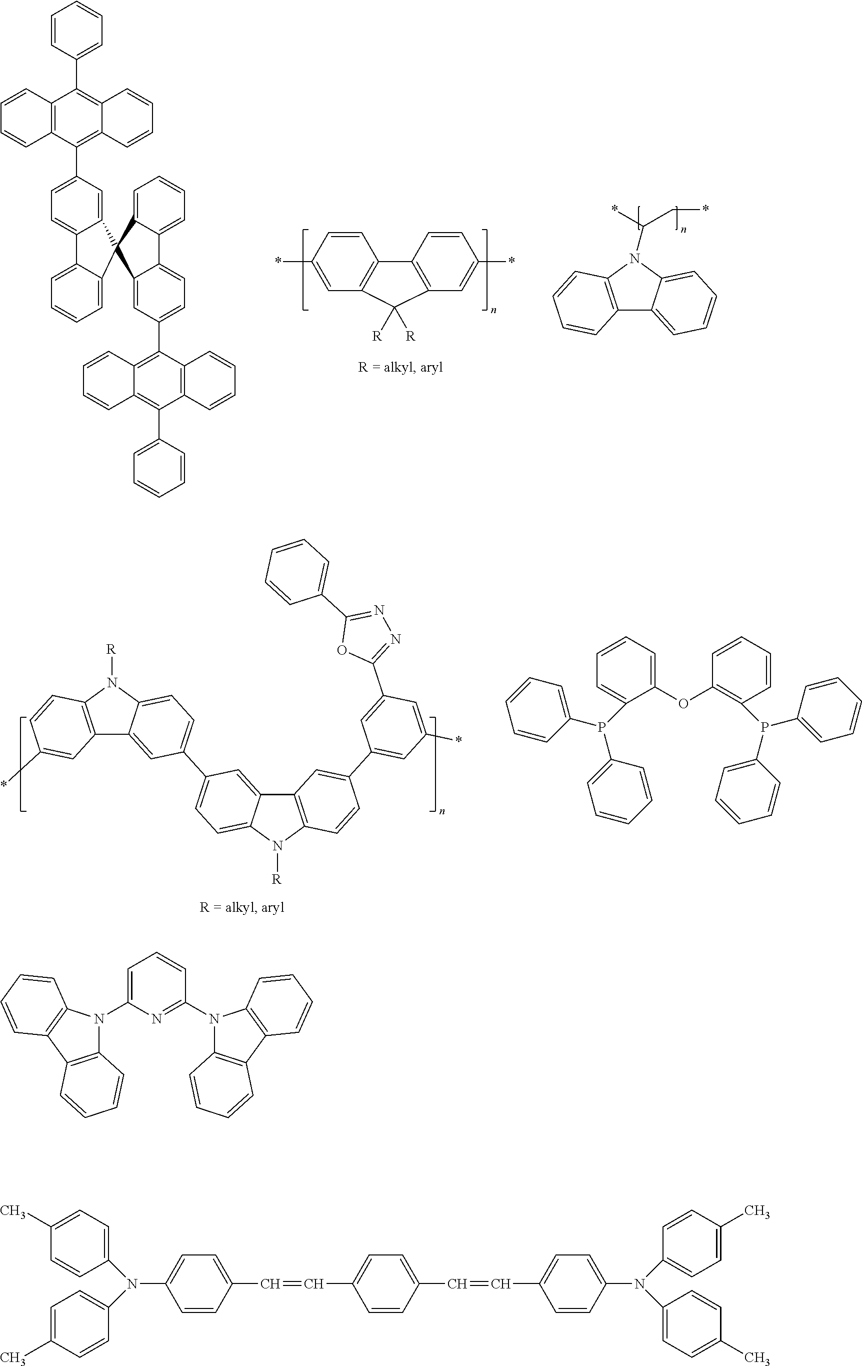

Next, preferred compounds for use as a hole injection material are mentioned below.

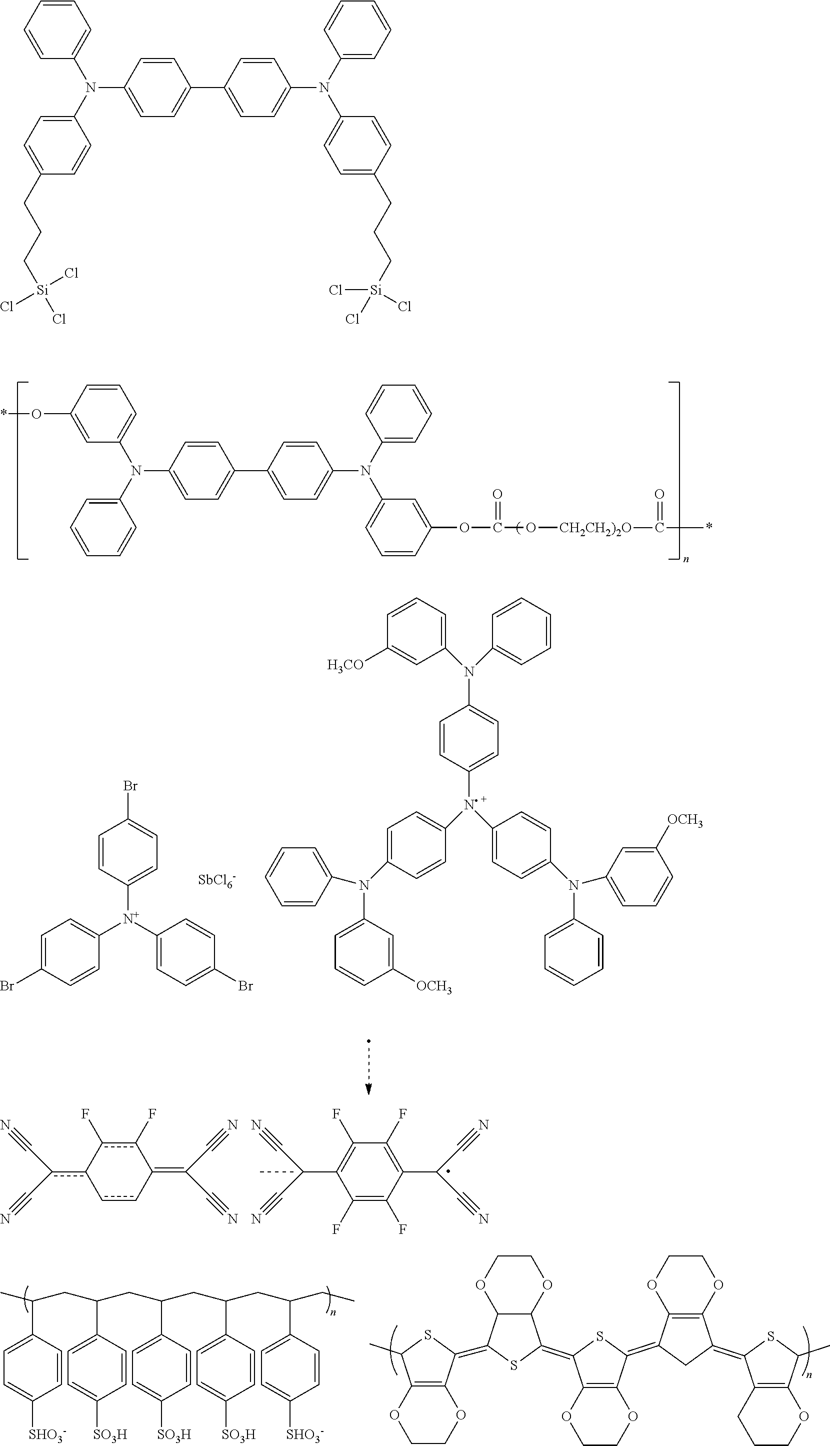

Next, preferred compounds for use as a hole transport material are mentioned below.

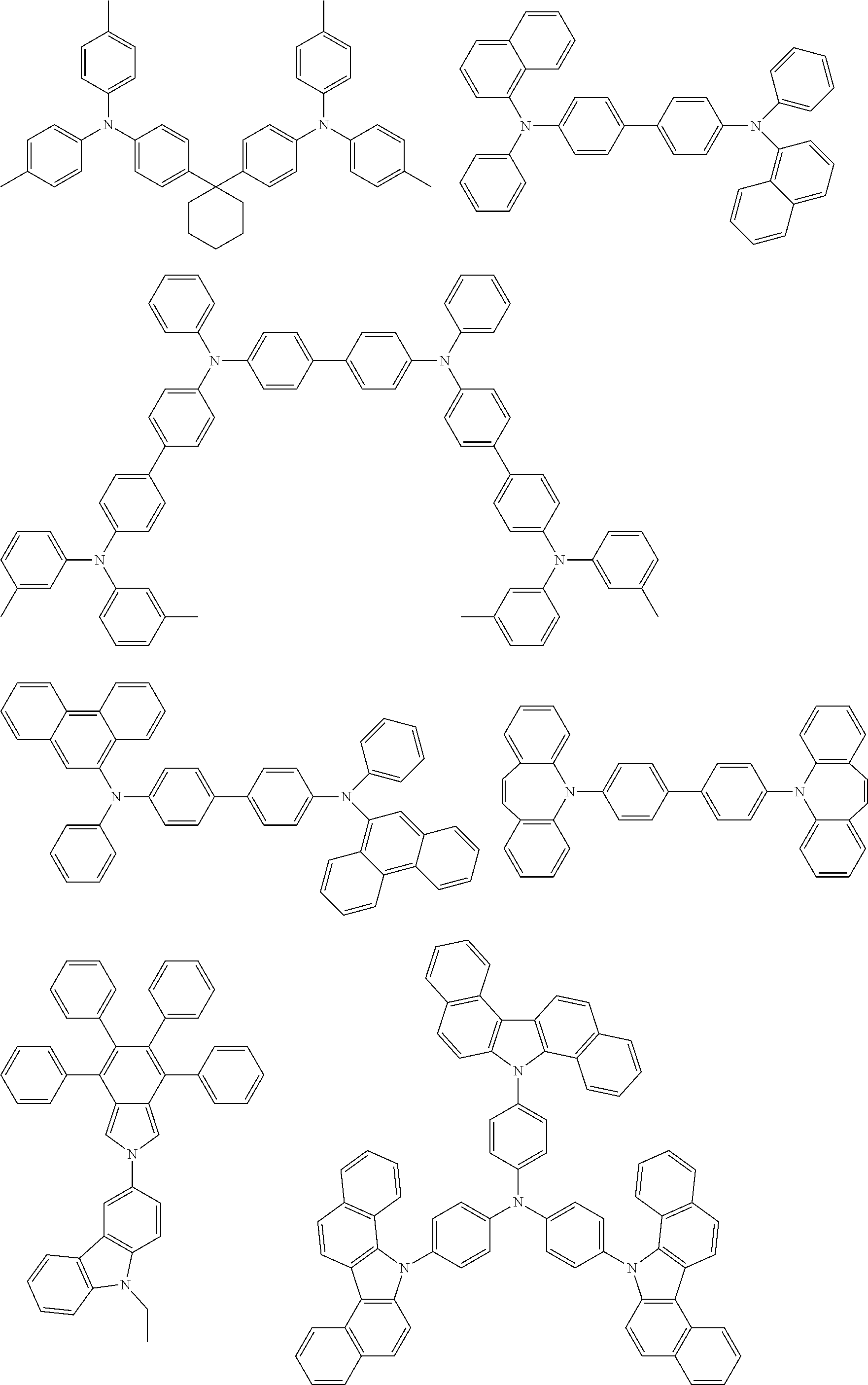

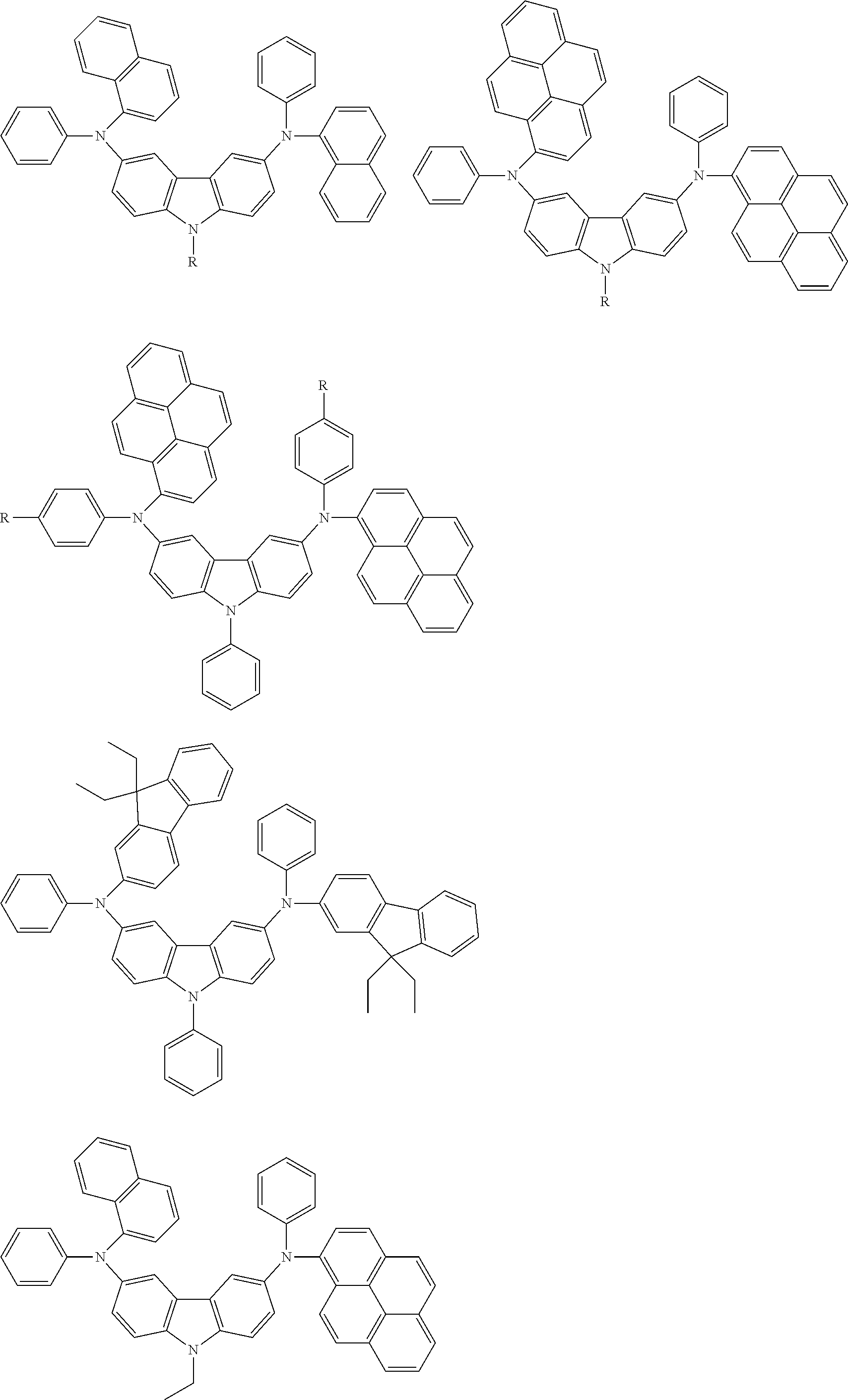

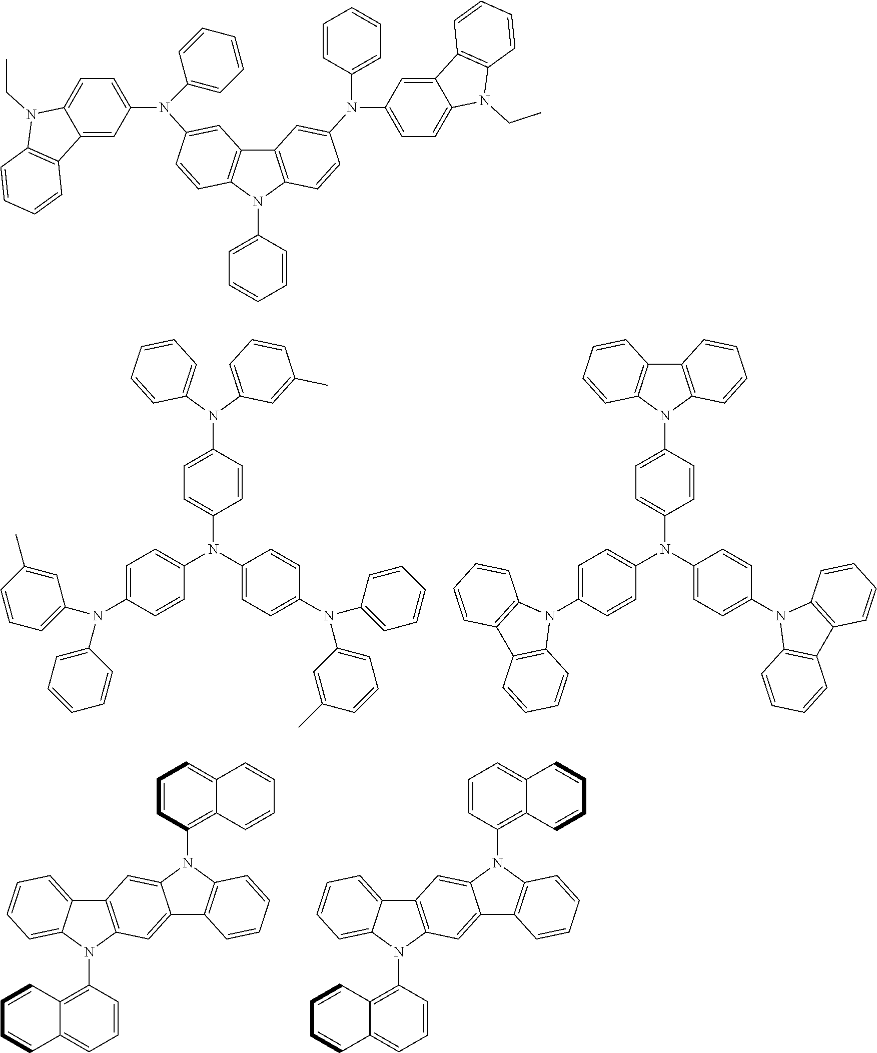

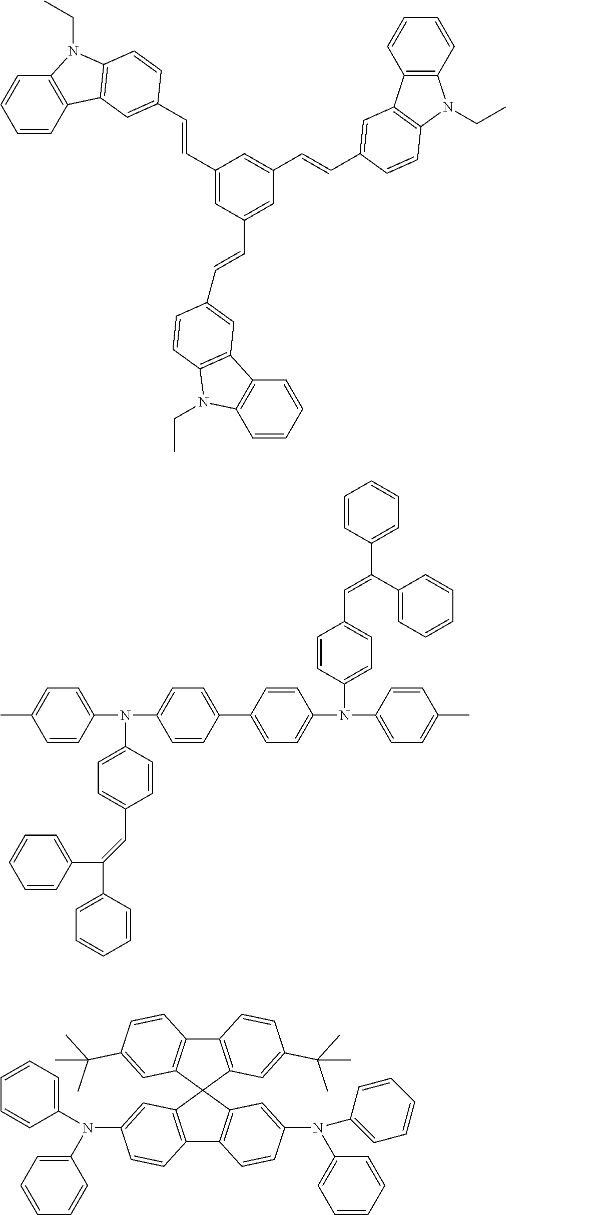

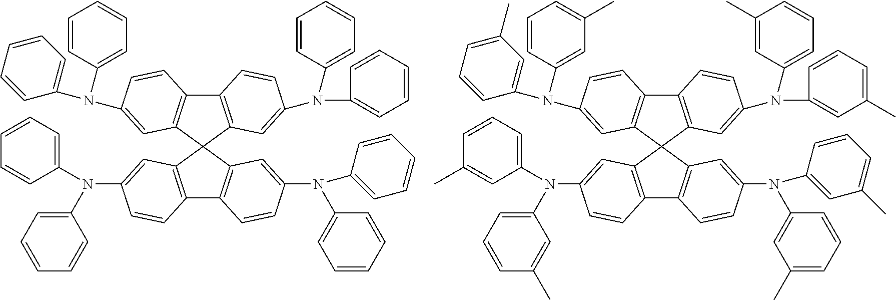

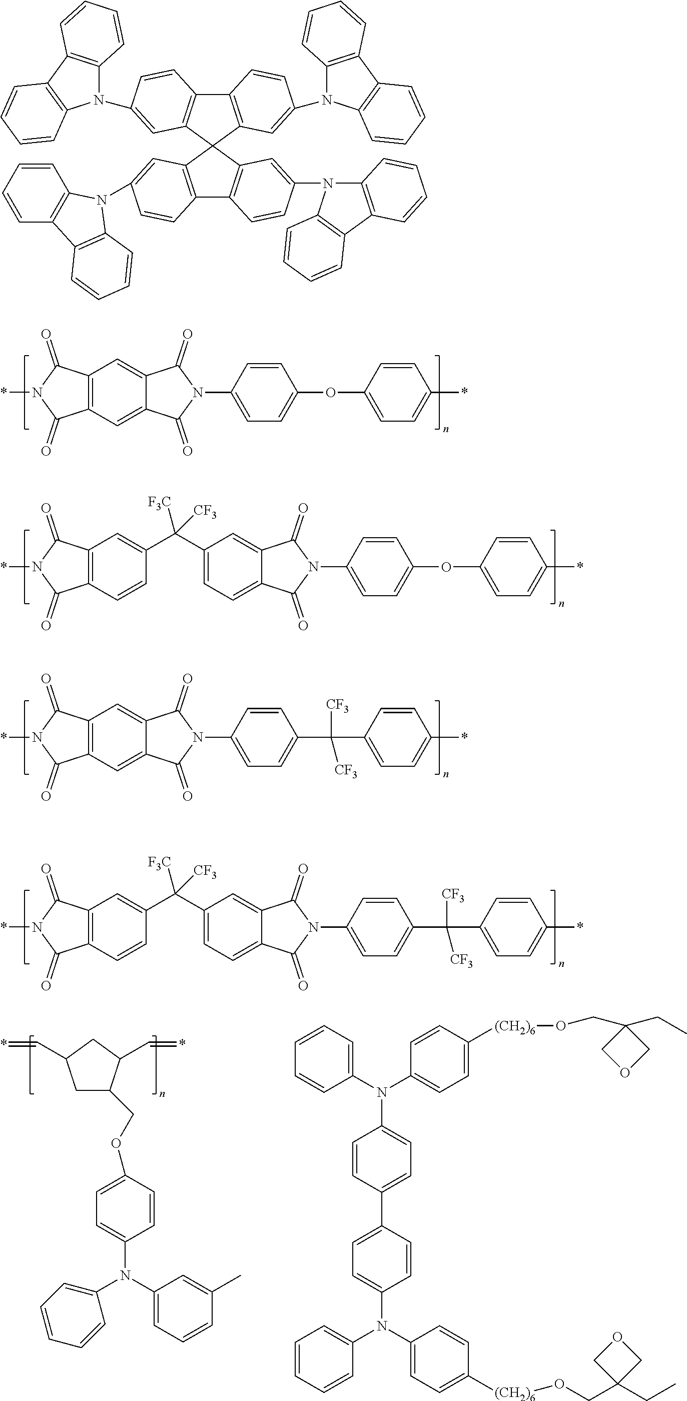

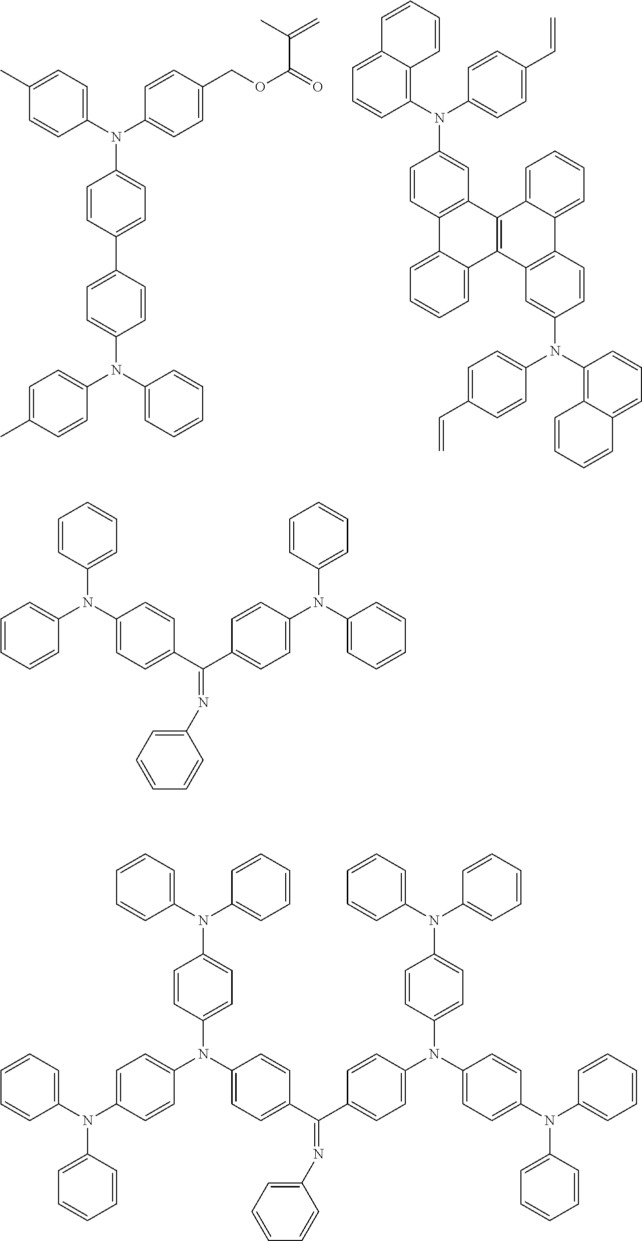

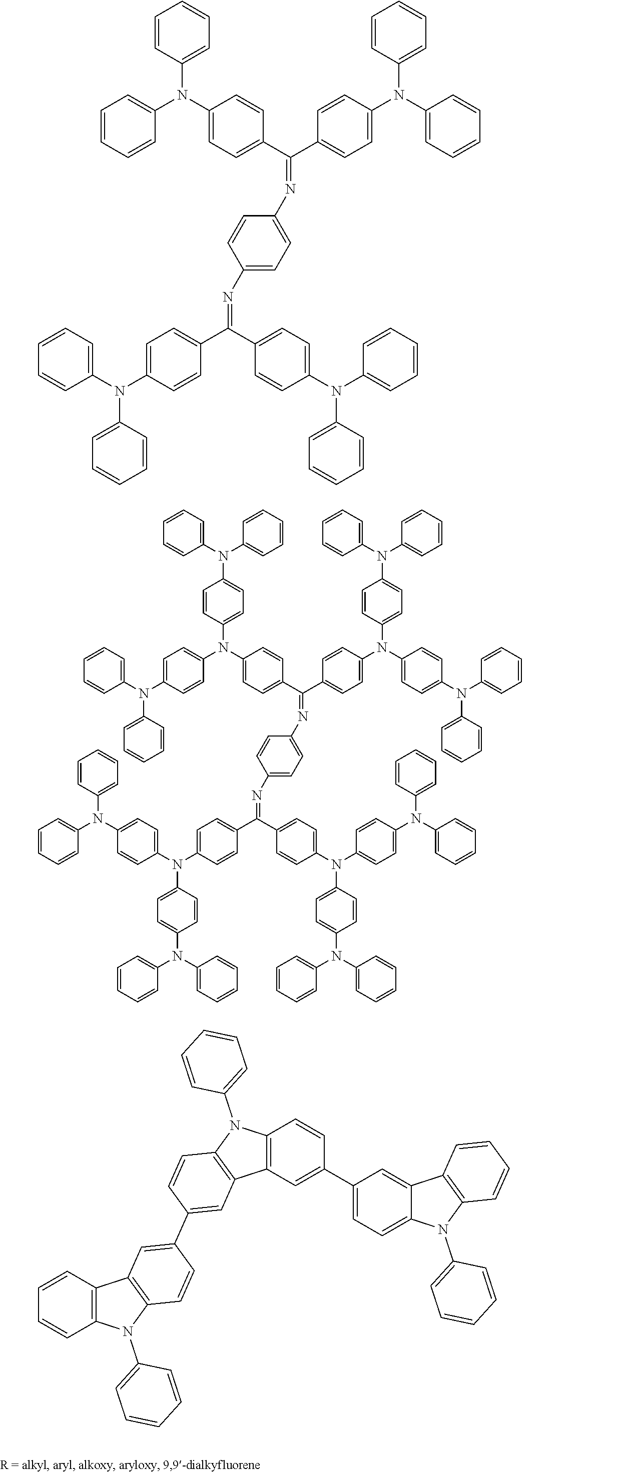

Next, preferred compounds for use as an electron blocking material are mentioned below.

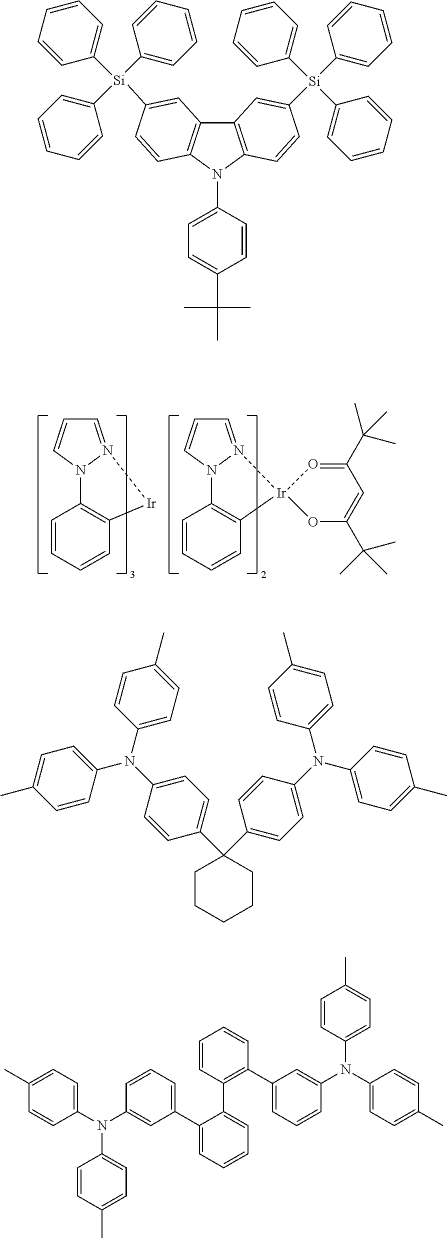

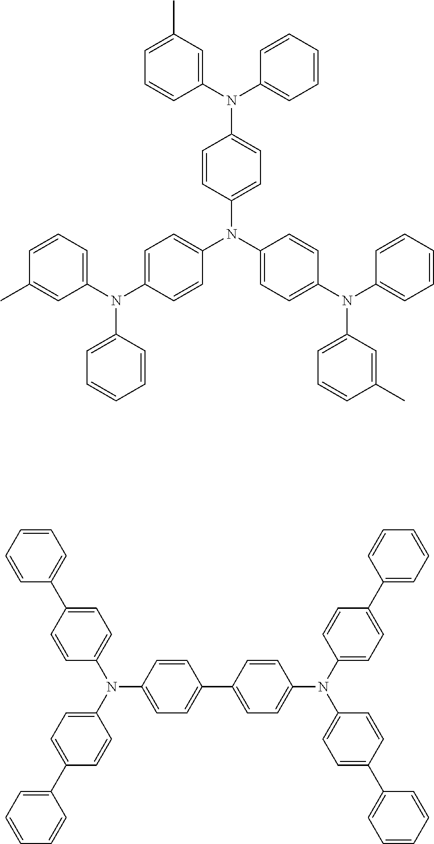 [SMILES: Cc1cccc(N(c2ccccc2)c2ccc(N(c3ccc(N(c4ccccc4)c4cccc(C)c4)cc3)c3ccc(N(c4ccccc4)c4cccc(C)c4)cc3)cc2)c1 ; c1ccc(-c2ccc(N(c3ccc(-c4ccccc4)cc3)c3ccc(-c4ccc(N(c5ccc(-c6ccccc6)cc5)c5ccc(-c6ccccc6)cc5)cc4)cc3)cc2)cc1]

Next, preferred compounds for use as a hole blocking material are mentioned below.

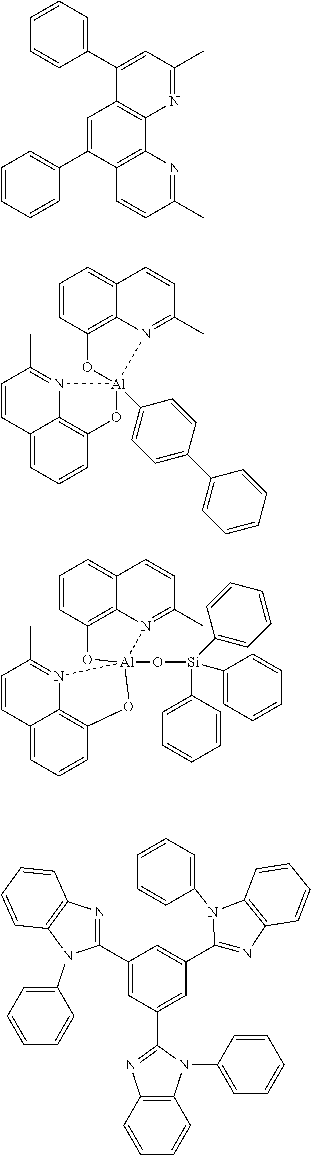

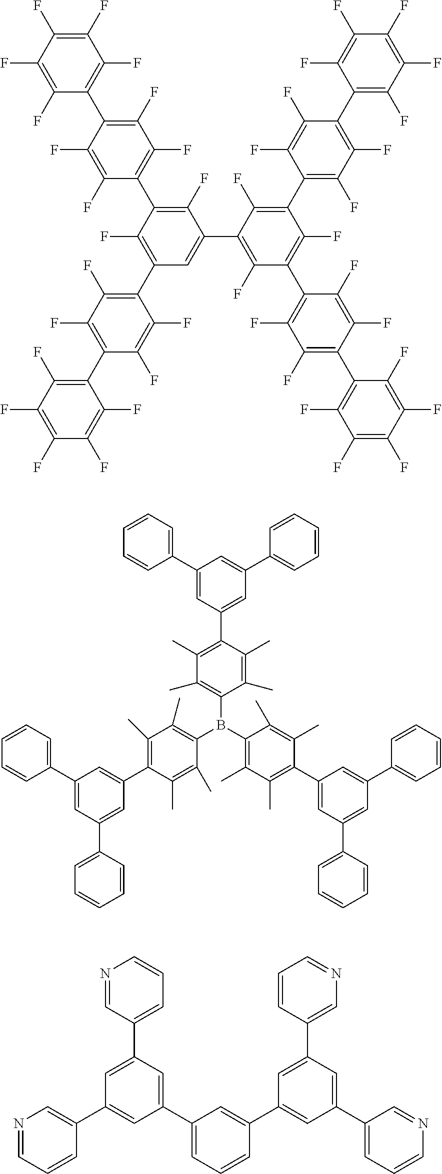 [SMILES: Fc1c(F)c(F)c(-c2c(F)c(F)c(-c3cc(-c4c(F)c(-c5c(F)c(F)c(-c6c(F)c(F)c(F)c(F)c6F)c(F)c5F)c(F)c(-c5c(F)c(F)c(-c6c(F)c(F)c(F)c(F)c6F)c(F)c5F)c4F)c(F)c(-c4c(F)c(F)c(-c5c(F)c(F)c(F)c(F)c5F)c(F)c4F)c3F)c(F)c2F)c(F)c1F ; Cc1c(C)c(-c2cc(-c3ccccc3)cc(-c3ccccc3)c2)c(C)c(C)c1B(c1c(C)c(C)c(-c2cc(-c3ccccc3)cc(-c3ccccc3)c2)c(C)c1C)c1c(C)c(C)c(-c2cc(-c3ccccc3)cc(-c3ccccc3)c2)c(C)c1C ; c1cncc(-c2cc(-c3cccnc3)cc(-c3cccc(-c4cc(-c5cccnc5)cc(-c5cccnc5)c4)c3)c2)c1]

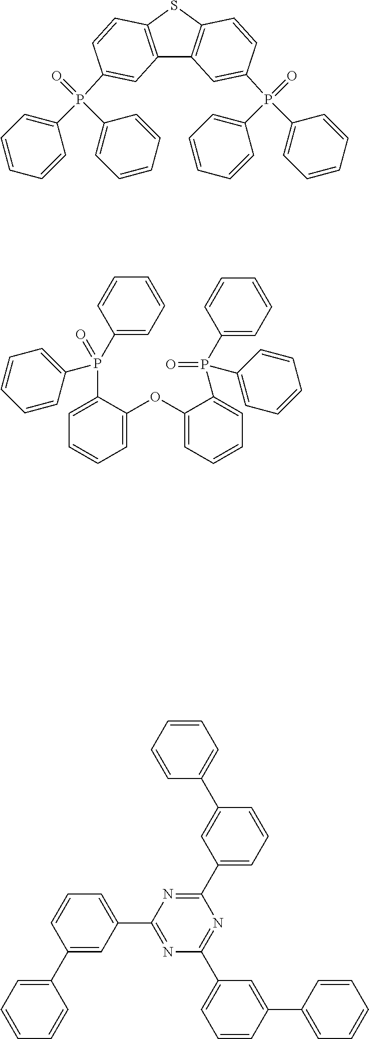 [SMILES: O=P(c1ccccc1)(c1ccccc1)c1ccc2sc3ccc(P(=O)(c4ccccc4)c4ccccc4)cc3c2c1 ; O=P(c1ccccc1)(c1ccccc1)c1ccccc1Oc1ccccc1P(=O)(c1ccccc1)c1ccccc1 ; c1ccc(-c2cccc(-c3nc(-c4cccc(-c5ccccc5)c4)nc(-c4cccc(-c5ccccc5)c4)n3)c2)cc1]

Next, preferred compounds for use as an electron transport material are mentioned below.

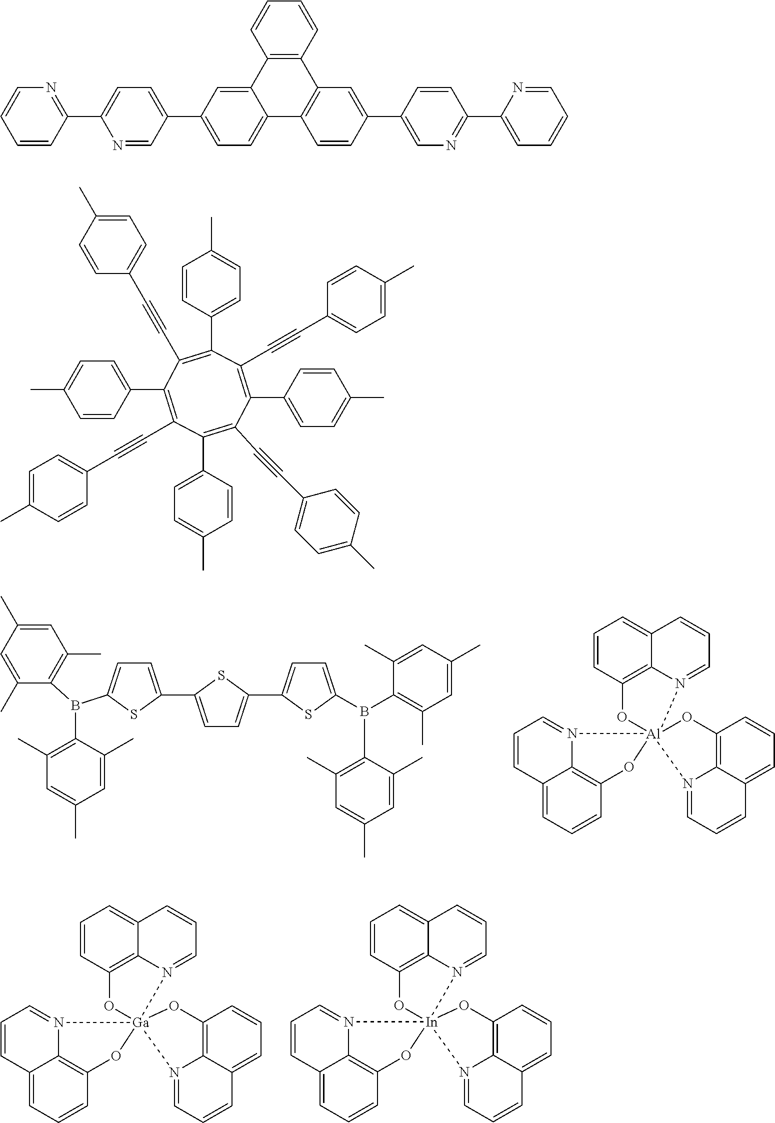

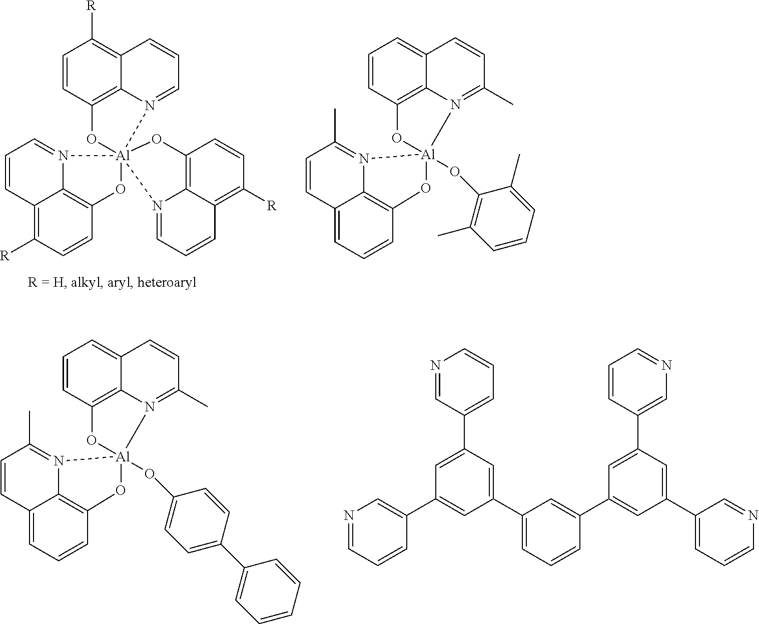

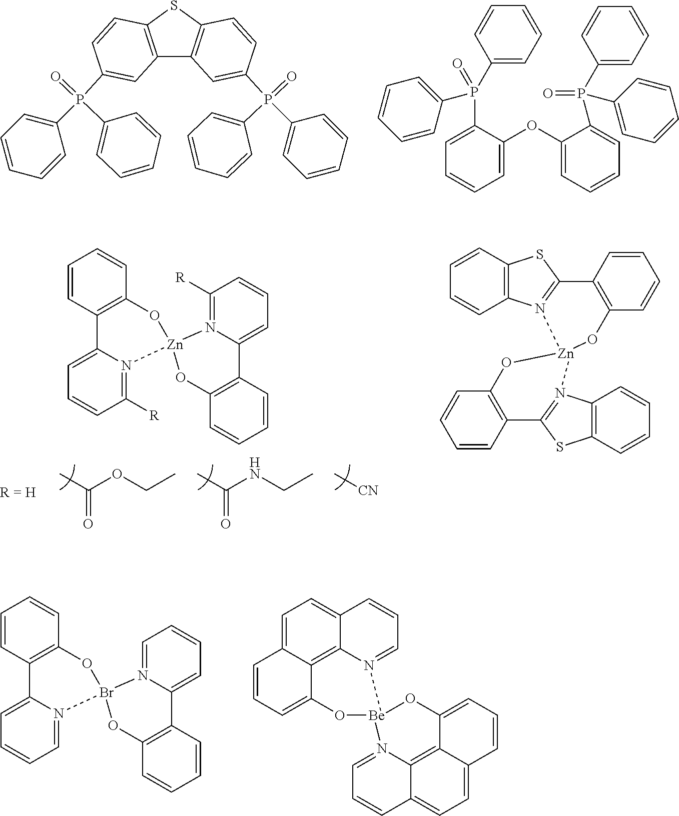

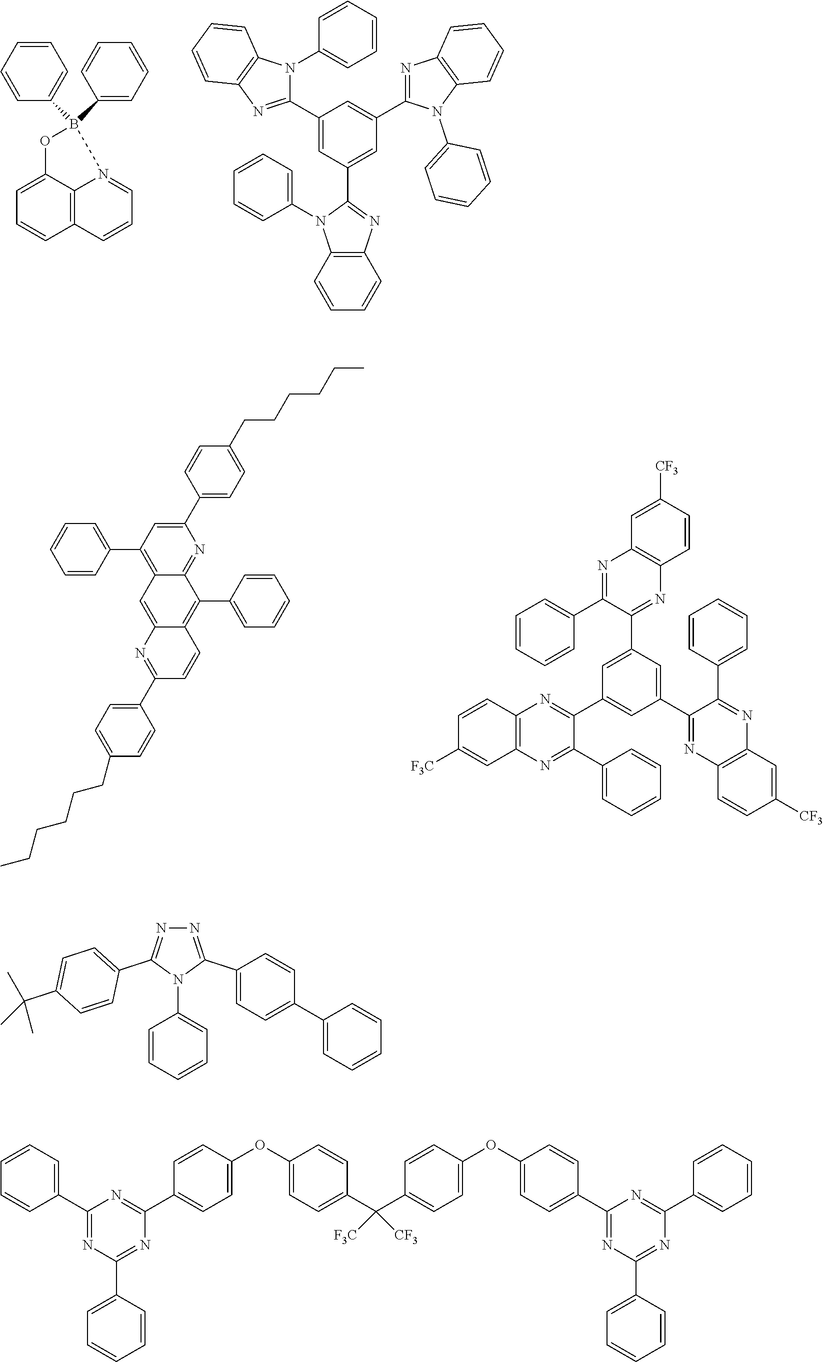

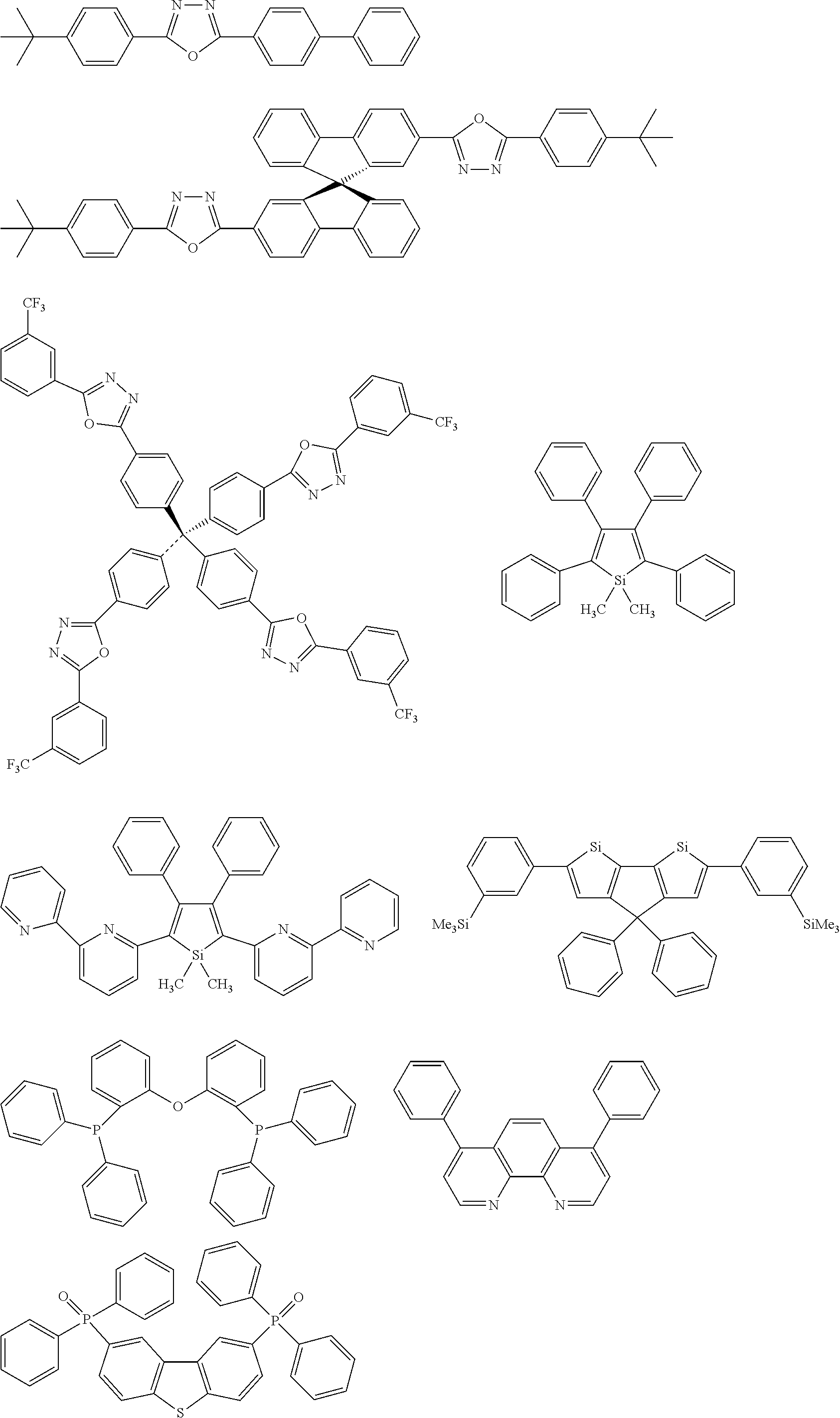

Next, preferred compounds for use as an electron injection material are mentioned below.

LiF, CsF

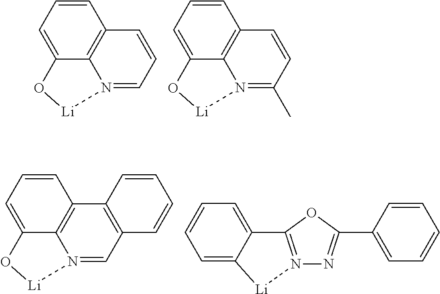

Further, preferred compounds for use as additional materials are mentioned below. For example, these are considered to be added as a stabilization material.

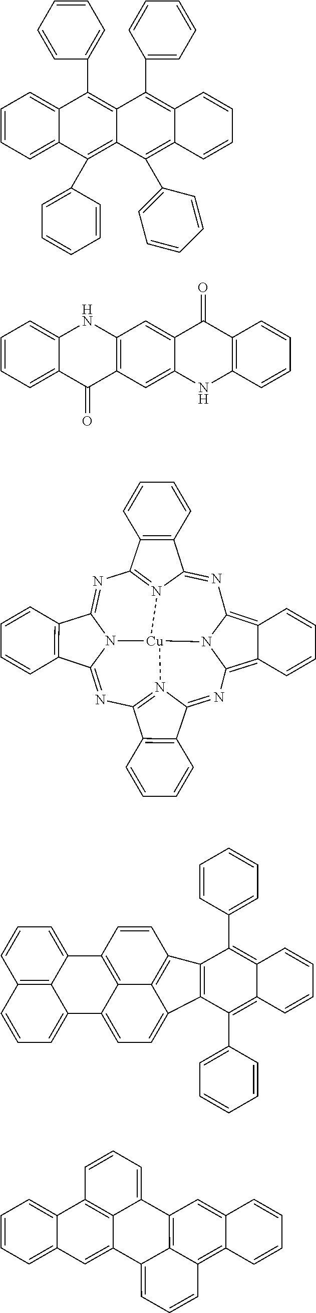

The organic electroluminescent device thus produced by the aforementioned method emits light on application of an electric field between the anode and the cathode of the device. In this case, when the light emission is caused by the excited singlet energy, light having a wavelength that corresponds to the energy level thereof may be confirmed as fluorescent light and delayed fluorescent light. When the light emission is caused by the excited triplet energy, light having a wavelength that corresponds to the energy level thereof may be confirmed as phosphorescent light. The normal fluorescent light has a shorter light emission lifetime than the delayed fluorescent light, and thus the light emission lifetime may be distinguished between the fluorescent light and the delayed fluorescent light.

On the other hand, the phosphorescent light may substantially not be observed with a normal organic compound such as the compound of the present invention at room temperature since the excited triplet energy is converted to heat or the like due to the instability thereof, and is immediately deactivated with a short lifetime. The excited triplet energy of the normal organic compound may be measured by observing light emission under an extremely low temperature condition.

The organic electroluminescent device of the invention may be applied to any of a single device, a structure with plural devices disposed in an array, and a structure having anodes and cathodes disposed in an X-Y matrix. According to the present invention using a compound capable of emitting delayed fluorescence and capable of undergoing intramolecular proton transfer in a light-emitting layer, an organic light-emitting device having a markedly improved light emission efficiency can be obtained. The organic light-emitting device such as the organic electroluminescent device of the present invention may be applied to a further wide range of purposes. For example, an organic electroluminescent display apparatus may be produced with the organic electroluminescent device of the invention, and for the details thereof, reference may be made to S. Tokito, C. Adachi and H. Murata, “Yuki EL Display” (Organic EL Display) (Ohmsha, Ltd.). In particular, the organic eiectroluminescent device of the invention may be applied to organic electroluminescent illumination and backlight which are highly demanded.

In addition, the organic light-emitting device of the present invention may also be an organic light-emitting transistor. An organic light-emitting transistor is, for example, so configured that a gate electrode is layered on an active layer serving also as a light-emitting layer, via a gate insulation layer, and a source electrode and a drain electrode are connected to the active layer. Using the compound capable of emitting delayed fluorescence and capable of undergoing intramolecular proton transfer as the active layer of such an organic light-emitting transistor, an organic light-emitting transistor excellent both in carrier mobility and emission performance can be realized.

### EXAMPLES

The features of the present invention will be described more specifically with reference to Synthesis Examples and Examples given below. The materials, processes, procedures and the like shown below may be appropriately modified unless they deviate from the substance of the invention. Accordingly, the scope of the invention is not construed as being limited to the specific examples shown below. The light emission characteristics were evaluated using an ultraviolet/visible light/near-infrared spectrophotometer (by PerkinElmer Corp., LAMBDA950-PKA.), a fluorescence spectrophotometer (by Horiba, Ltd., FluoroMax-4), a multichannel spectroscope (by Hamamatsu Photonics K.K., PMA-12C10027-01), a photoexcitation absolute luminescent quantum efficiency measuring system (by Hamamatsu Photonics K.K., C9920PMMA-11), a fluorescent lifetime measuring device (by Hamamatsu Photonics K.K., C11367-25), and a streak camera (by Hamamatsu Photonics K.K., U8167-1), In Examples, fluorescence having an emission lifetime of 100 ns or less is judged as instantaneous fluorescence, and fluorescence having an emission lifetime 0.1 μs or more is judged as delayed fluorescence.

The HOMO (highest occupied molecular orbital) energy level and the LUMO (lowest unoccupied molecular orbital) energy level were measured using an electrochemical analyzer (by BAS Corp.) in cyclic voltammetry and differential pulse voltammetry using a 0.1 mM ferrocene solution as an external standard.

A difference ΔE_{ST} between a lowest excited singlet energy level (E_{S1}) and a lowest excited triplet energy level (E_{T1}) was measured as follows.

### Difference ΔE_{ST} between Lowest Excited Singlet Energy Level (E_{S1}) and Lowest Excited Triplet Energy Level (E_{T1}) in Toluene or PMMA

For the difference ΔE_{ST} between a lowest excited singlet energy level (E_{S1}) of a compound and a lowest excited triplet energy level (E_{T1}) thereof in toluene or PMMA, a lowest excited singlet energy level (E_{S1}) of a compound and a lowest excited triplet energy level (E_{T1}) thereof were calculated according to the methods mentioned below, and the difference was determined as ΔE_{ST}=E_{S1}−E_{T1}.

(1) Lowest excited singlet energy level (E_{S1})

A toluene solution containing the compound to be analyzed (concentration: 1×10⁻⁵M), or a PMMA film containing the compound to be analyzed, as formed on a silicon substrate (compound concentration: 0.1 mol %, thickness 100 nm) were prepared as measurement samples, and the fluorescent spectrum of each sample was measured at room temperature (300 K). Specifically, the emission immediately after excitation light incidence up to 100 nanoseconds after the light incidence was integrated to draw a fluorescent spectrum on a graph where the vertical axis indicates the emission intensity and the horizontal axis indicates the wavelength. A tangent line was drawn to the rising of the emission spectrum on the short wavelength side, and the wavelength value λedge [nm] at the intersection between the tangent line and the horizontal axis was read. The wavelength value was converted into an energy value according to the following conversion expression to calculate E_{S1} .

Conversion Expression: E_{S1} [eV]=1239.85/λedge

(2) Lowest excited triplet energy level (E_{T1})

The same sample as that for measurement of the lowest excited singlet energy level (E_{S1}) was cooled to 5 [K], and the sample for phosphorescence measurement was irradiated with excitation light (337 nm), and using a streak camera, the phosphorescence intensity thereof was measured. The emission in 1 millisecond after excitation light incidence up to 10 milliseconds after the light incidence was integrated to draw a phosphorescent spectrum on a graph where the vertical axis indicates the emission intensity and the horizontal axis indicates the wavelength. A tangent line was drawn to the rising of the phosphorescent spectrum on the short wavelength side, and the wavelength value λedge [nm] at the intersection between the tangent line and the horizontal axis was read. The wavelength, value was converted into an energy value according to the following conversion expression to calculate E_{T1}.

Conversion Expression: E_{T1} [eV]=1239.85/λedge

The tangent line to the rising of the phosphorescent spectrum on the short wavelength side was drawn as follows. While moving on the spectral curve from the short wavelength side of the phosphorescent spectrum toward the maximum value on the shortest wavelength side among the maximum values of the spectrum, a tangent line at each point on the curve toward tile long wavelength side was taken into consideration. With rising thereof (that is, with increase in the vertical axis), the inclination of the tangent line increases. The tangent line drawn at the point at which the inclination value has a maximum value was referred to as the tangent line to the rising on the short wavelength side of the phosphorescent spectrum.

The maximum point having a peak intensity of 10% or less of the maximum peak intensity of the spectrum was not included in the maximum value on the above-mentioned shortest wavelength side, and the tangent line drawn at the point which is closest to the maximum value on the shortest wavelength side and at which the inclination value has a maximum value was referred to as the tangent lint to the rising on the short wavelength side of the phosphorescent spectrum.

The sample in the form of a toluene solution was measured using a phosphorescence measuring device at 77 [K].

### Difference ΔE_{T1} between Lowest Excited Singlet Energy Level E_{Si} and Lowest Excited Triplet Energy Level E_{T1} in mCBP

The difference ΔE_{ST} between the lowest excited singlet energy level E_{S1} and the lowest excited triplet energy level E_{T1} of a compound in mCBP was determined from the inclination a on a correlation diagram of T⁻¹ and In (k_{RISC}) measured in an experiment in which ΔE_{S1} is the activation energy Eₐ in the Arrhenius equation.

Specifically, when both sides of the Arrhenium equation expressed by the following equation (1) are converted into natural logarithms, then the following expression (2) is given.

### Arrhenium Equation:

$$\begin{matrix}k = A exp ( - \frac{{E}_{a}}{RT} ) & ( 1 ) \\ ln k = ln A exp ( - \frac{{E}_{a}}{RT} ) = ln A + ln exp ( - \frac{{E}_{a}}{RT} ) = - \frac{{E}_{a}}{R} {T}^{- 1} + ln A & ( 2 )\end{matrix}$$

In the expressions (1) and (2), k represents a reaction rate constant. A represents a frequency factor, Eₐ represents an activation energy, R represents a gas constant, and T represents an absolute temperature. Here, the gas constant with one molecule is 0.86173312×10⁻⁴ (eVK⁻¹).

From the equation (2), it is known that T⁻¹ and In k can be expressed as a linear function with an inclination −Eₐ/R. Accordingly, where the inclination of the correlation diagram of T⁻¹ and In k is a, the activation energy Eₐ can be approximately expressed by the following equation (4).

Eₐ=−a×0.8617×10⁻⁴ (4)

Here, in reverse intersystem crossing from an excited singlet state to an excited triplet state, ΔE_{ST} can be considered to be an activation energy (height of potential barrier), and therefore the following expression (5) is established where Eₐ in the expression (4) is replaced with ΔE_{ST}.

ΔE_{ST}=−a×0.8617×10⁻⁴ (5)

In the present Examples, a co-evaporation film of a subject compound and mCBP was so formed that the concentration of the subject compound therein could be 3% by weight, and the transient decay curve of emission thereof was measured at a different temperature in a range of 10 to 300 K to provide a correlation diagram between T⁻¹ and in (k_{RISC}) (where k_{RISC} is a rate constant of reverse intersystem crossing). From the inclination a on the correlation diagram, ΔE_{ST} was derived according to the equation (5).

### (Synthesis Example 1) Synthesis of Compound 1

3-(4,4,5,5-tetramethyl-1,3,2-dioxaboroooran-2-yl)-1,1,5,5,9,9-hexamethyl-13-azatriangulene (197 mg, 0.40 mmol) and 2-bromo-4,6-diphenyl-1,3,5-triazine (140 mg, 0.45 mmol) were dissolved in 30 mL of toluene in a nitrogen atmosphere. Further, 55 mL of toluene was added thereto and an aqueous 2 M sodium carbonate solution (15 mL) containing 3 drops of methyltrioctylammonium chloride and tetrakis(triphenylphosphine palladium(0) (56 mg, 2 mol %) were added, and stirred at 65° C. in a nitrogen atmosphere for 3 days. The aqueous layer was washed three times with toluene, the collected organic layer was dried with magnesium sulfate, and the solvent was evaporated away under reduced pressure. The resultant crude product was purified through silica gel column chromatography using a mixed solvent of n-hexane/dichloroethane=3/1 as an eluent to give a pale yellow solid of a compound 1. ¹H-NMR (CDCl₃, 400 MHz, δ): 1.69 (s, 6H), 1.777 (s, 12H), 7.20 (t, 2H, J=7.5 Hz), 7.43 (dd, 2H , J=7.5, 1.5), 7.47 (dd, 2H, J=7.5, 1.5), 7.61 (m, 6H), 8.80 (m, 6H), MS(ASAP): m/z 598 ([M+H]⁺).

### (Synthesis Example 2) Synthesis of Compound 2

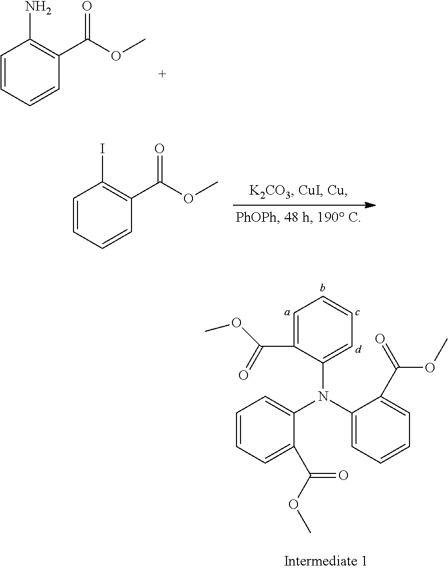

A mixture of methyl anthranilate (41.1 mL, 0.318 mol), methyl 2-iodo-benzoate (13 mL, 0.909 mol), potassium carbonate (100 g, 0727 mol), copper(I) iodide (5.89 g, 0.0309 mol) and copper (4.04 g, 0.0636 mol) was put into 370 mL of diphenyl ether, and stirred at 190° C. in a nitrogen atmosphere for 76 hours. The reaction liquid was filtered through Celite to remove solids, and then processed for liquid-liquid separation using chloroform. The resultant organic layer was dried with sodium sulfate, sodium sulfate was removed through filtration, and then the solvent was evaporated away from the organic layer with an evaporator to give a pale beige solid of an intermediate 1. The resultant solid was recrystallized with ethyl acetate to give a pale yellow solid of the intermediate 1 in a yield amount of 98.7 g and a yield of 74%.

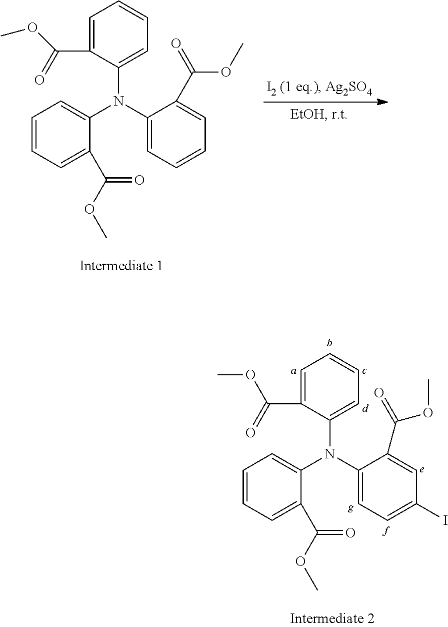

A mixture of the intermediate 1 (1.00 g, 2.39 mmol) and silver(I) sulfate (0.750 g, 2.39 mmol) was suspended in ethanol (50 mL), and iodine (0.608 g, 2.39 mmol) dissolved in ethanol (100 mL) was gradually and dropwise added thereto with cooling with ice, and then stirred at room temperature for 15 hours. The reaction liquid was filtered through Celite to remove solids, and the solvent was evaporated away with an evaporator. The precipitated solid was purified through silica gel column chromatography using dichloromethane as an eluent to give a beige powdery solid of an intermediate 2 in a yield amount of 0.450 g and a yield of 35%.

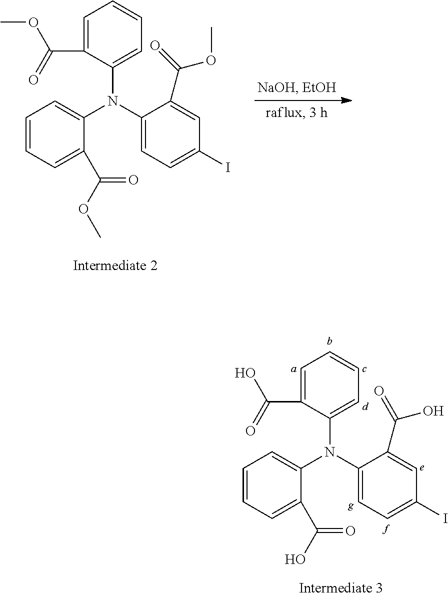 [SMILES: COC(=O)c1ccccc1N(c1ccccc1C(=O)OC)c1ccc(I)cc1C(=O)OC ; O=C(O)c1ccccc1N(c1ccccc1C(=O)O)c1ccc(I)cc1C(=O)O]

A mixture prepared by adding sodium hydroxide (0.829 g, 20.7 mmol) to the intermediate 2 (0.801 g, 1.47 mmol) was put into a mixed solution (6.5 mL) of ethanol/water=1/1, and heated under reflux for 3 hours, and then hydrochloric acid was drop vise added thereto to adjust the pH of the reaction solution to about 1. The precipitated solid was collected through filtration under reduced pressure, and dried under reduced pressure to give a white powder of an intermediate 3 in a yield amount of 0.659 g and a yield of 89%.

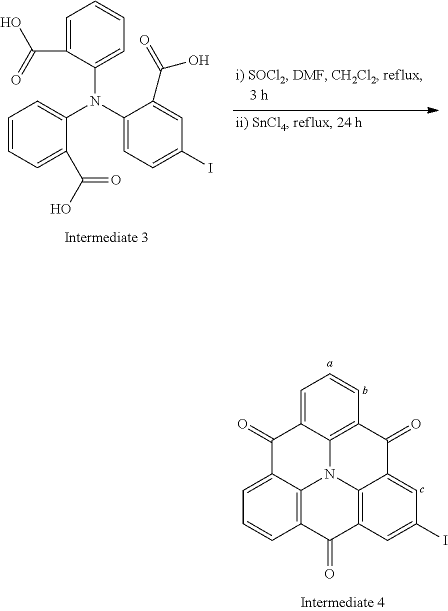 [SMILES: O=C(O)c1ccccc1N(c1ccccc1C(=O)O)c1ccc(I)cc1C(=O)O ; O=c1c2cccc3c(=O)c4cc(I)cc5c(=O)c6cccc1c6n(c23)c45]

The intermediate 3 (0.600 g, 1.19 mmol), thionyl chloride (2.57 mL, 35.7 mmol) and N,N-dimethylformamide (0.30 mL) were put into dewatered dichloromethane (45 mL), and heated under reflux for 3 hours in a nitrogen atmosphere, then tin(IV) tetrachloride (2.52 mL, 21.4 mmol) was added thereto and further heated under reflux for 18 hours. After the reaction, an aqueous sodium hydroxide solution was gradually and dropwise added to the reaction liquid with stirring, and stirred at room temperature for 1 hour, The resultant solid was collected through suction filtration, dried under reduced pressure, and recrystallized with nitrobenzene. Accordingly, a yellow solid of an intermediate 4 was obtained in a yield amount of 0.287 g and a yield of 54%.

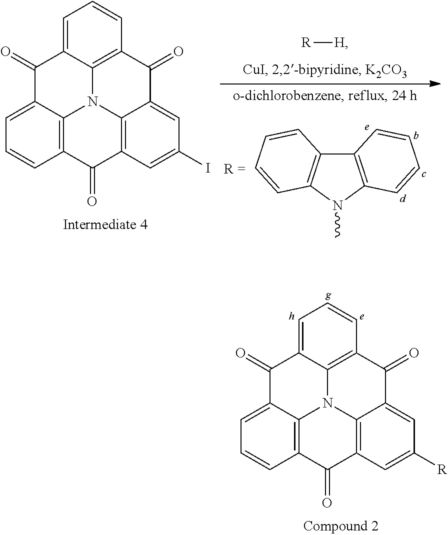

A mixture of the intermediate 4 (0.200 g, 0.445 mmol), 9H-carbazole (0.089 g, 0.534 mmol), copper(I) iodide (0.0169 g, 0.089 mmol), 2,2-bipyridine (0.0138 g, 0.089 mmol) and potassium carbonate (0.741 g, 4.45 mmol) was put into o-dichlorobenzene (45 mL), and heated under reflux for 44 hours in a nitrogen atmosphere. After the reaction, the reaction solution was cooled to around room temperature, and diluted with dichloromethane added thereto, and the solids were removed through filtration under reduced pressure. The solvent was evaporated away under reduced pressure from the resultant filtrate, and the precipitated solid was roughly purified through silica gel column chromatography using chloroform as an eluent. The roughly purified product was further purified through sublimation to give the intended compound 2 as an orange solvent in a yield amount of 0.0301 g and a yield of 14%.

¹H-NMR (500 MFTz, CDCl₃): δ=9.29 (s, 2H, ArHₑ), 9.11 (dd, 2H, J=7.5 Hz, 2.0 Hz, ArH_{f}), 9.08 (dd, 2H, J=7.5 Hz, 2.0 Hz, ArHₕ), 8.19 (d, 2H, J=8.0 Hz, 2.0 Hz, ArHₐ), 7.91 (dd, 2H, J=7.5 Hz, 7.5 Hz, ArH_{g}), 7.56 (d, 2H, 8.0 Hz, ArH_{d}), 7.47 (ddd, 2H, J=7.0 Hz, 7.0 Hz, 1.0 Hz, ArH_{c}), 7.37 (ddd, 2H, J=7.5 Hz, 7.0 Hz, ArH_{b}), MS (ASAP) m/z 488.41 [M⁺−H].

### (Synthesis Example 3) Synthesis of Compound 3

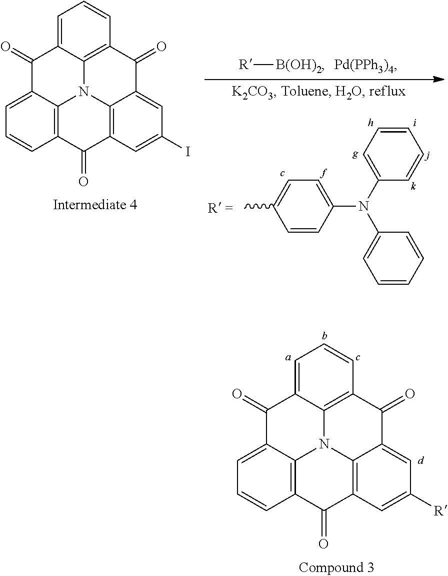

Toluene (5 mL) was added to a mixture of the intermediate 4 (0.0906 g, 0.200 mmol), triphenylamineboronic acid (0.0589 g, 0.200 mmol), and tetrakis(triphenylphosphine)palladium(0) (0.0106 g, 0.0095 mmol), and then potassium carbonate (1.42 g, 1.03 mmol) and pure water (5 mL) were added thereto and heated under reflux for 30 hours in a nitrogen atmosphere. After the reaction, the reaction solution was processed for liquid-liquid separation using chloroform, and the resultant organic layer was dried with magnesium sulfate, then magnesium sulfate was removed through filtration, and the solvent was removed with an evaporator. The precipitated solid was roughly purified through silica gel column chromatography using chloroform as an eluent. The resultant roughly purified product was further purified through sublimation to give a red orange solid of a compound 3 in a yield amount of 0.0237 g and a yield of 21%.

¹H-NMR (500 MHz, CDCl₃): δ=9.25 (s, 2H, ArH_{d}), 9.07 (ddd, 4H, J=6.0 Hz, 6.0 Hz, 1.5 Hz, ArH_{a,c}), 7.87 (dd, 2H, J=7.5 Hz, 7.5 Hz, ArH_{b}), 7.77 (d, 2H, J=9.0 Hz, ArH_{c}), 7.32 (dd, 4H, J=8.0 Hz, 7.5 Hz, ArH_{h,j}), 7.23 (d, 2H, J=8.5 Hz, ArH_{f}) 7.19 (d, 2H, J=8.0 Hz, ArH_{g,k}), 7.10 (dd, 2H, J=7.5 Hz, 7.5 Hz, ArHᵢ), MS (ASAP) m/z 566.18 [M⁺].

### (Synthesis Example 4) Synthesis of compound 4

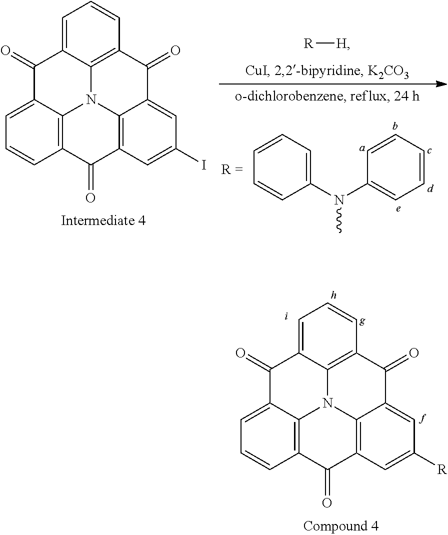

A mixture of the intermediate 4 (0.201 g, 0.445 mmol), diphenylamine (0.0898 g, 0.534 mmol), copper(1) iodide (0.0171 g, 0.089 mmol), 2,2-bipyridine (0.0133 g, 0.089 mmol) and potassium carbonate (0.742 g, 4.45 mmol) were put into o-dichlorobenzene (45 mL), and heated under reflux for 44 hours in a nitrogen atmosphere. After the reaction, the reaction solution was cooled to around room temperature, diluted with dichloromethane added thereto, and the solids were removed through filtration under reduced pressure. The solvent was evaporated away under reduced pressure from the resultant filtrate, and the precipitated solid was roughly purified through silica gel column chromatography using chloroform as an eluent. The resultant roughly purified product was further purified through silica gel permeation chromatography to give a red solid of a compound 4 in a yield amount of 0.0020 g and a yield of 0,9%,

¹H-NMR (500 MHz, CDCl₃): δ=9.03 (dd, 2H, J=7.5 Hz, 2.0 Hz, ArH_{g}), 8.98 (dd, 2H, J=7.5 Hz; 1.5 Hz, ArHᵢ), 8.66 (s, 2H, ArH_{f}), 7.82 (dd, 2H, J=8.0 Hz, 8.0 Hz, ArHₕ), 7.37 (dd, 4H, J=8.0 Hz, 7.5 Hz, ArH_{b,d}), 7.21 (d, 4H, 8.0 Hz, ArH_{a,e}), 7.18 (dd, 2H, J=7.5 Hz, ArH_{c}), MS (ASAP) m/z 490 [M⁺−H].

### (Synthesis Example 5) Synthesis of Compound 5

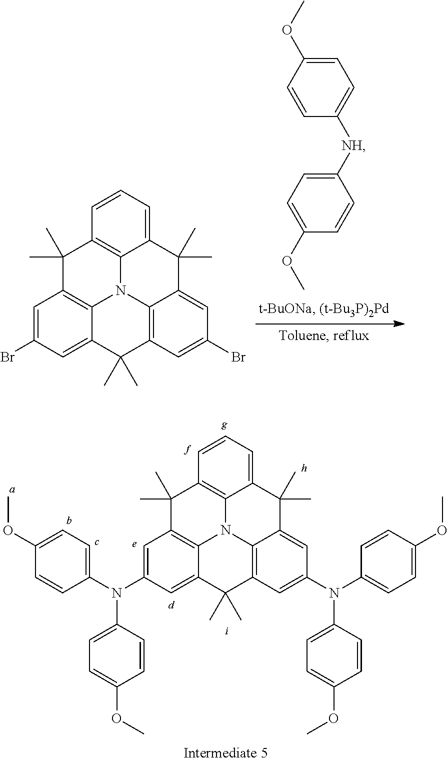 [SMILES: CC1(C)c2cccc3c2N2c4c1cc(Br)cc4C(C)(C)c1cc(Br)cc(c12)C3(C)C ; COc1ccc(Nc2ccc(OC)cc2)cc1 ; COc1ccc(N(c2ccc(OC)cc2)c2cc3c4c(c2)C(C)(C)c2cc(N(c5ccc(OC)cc5)c5ccc(OC)cc5)cc5c2N4c2c(cccc2C5(C)C)C3(C)C)cc1]

In a nitrogen stream, 0.7 g (1.34 mmol) of 2,6-dibromo-4,4,8,8,12,12-hexamethyl-8,12-dihydro-4H-benzo[1,9]quinolidine[3,4,5,6,7-defg]acridine, 0.92 g (4.02 mmol) of N,N-di(4-anisoly)amine, 0.48 g (5.00 mmol) of sodium t-butoxide and 15 mg (0.03 mmol) of bis(tri-t-butylphosphine)palladium(0) were heated under reflux in 50 mL of dewatered toluene for 48 hours, and the resultant reaction solution was filtered through Celite, then processed for liquid-liquid separation, dried on an anhydrous sodium sulfate, and the solvent was evaporated away to give a yellow reaction mixture. The intended product was purified through column chromatography (hexane/ethyl acetate) to give a yellow powder of an intermediate 5 in a yield amount of 0.9 g (1.10 mmol) and a yield of 82%.

¹H-NMR (500 MHz, toluene d-8): δ=7.34 (d, 2H, J=2.5 Hz, ArH_{dore}), 7.28 (d, 2H, J=2.5 Hz, ArH_{dore}), 7.21 (d, 1H, J=8.0 Hz, ArH_{f}), 7.17 (d, 8H, J=9.0 Hz, ArH_{c}), 6.98 (s, 1H, ArH_{g}), 6.74 (d, 8H, J=9.0 Hz, ArH_{f}), 3.36 (s, 12H, ArHₐ), 1.51 (s, 12H, ArHₕ), 1.45 (s, 6H, ArHᵢ).

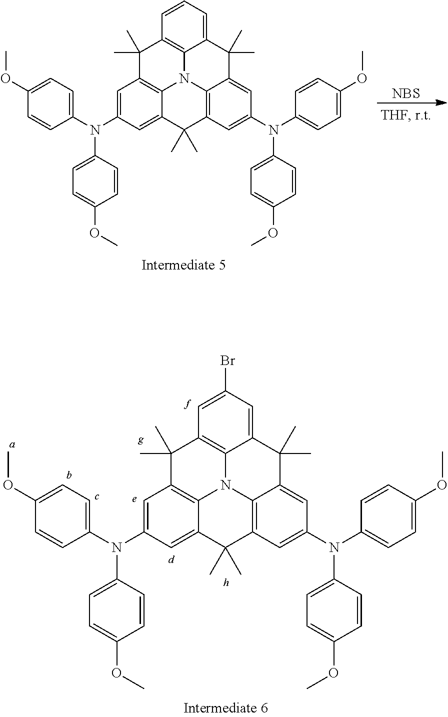

In a nitrogen stream, the intermediate 5 (410 mg, 0.5 mmol) was dissolved in tetrahydrofuran (20 mL), and with cooling with ice, N-bromosuccinimide (108 mg, 0.6 mmol) dissolved in tetrahydrofuran (10 mL) was gradually and dropwise added thereto. After stirred for 1 hour, this was further stirred overnight at room temperature. A small amount of an aqueous sodium thiosulfate solution was added to the resultant reaction solution, then processed for liquid-liquid separation, dried on anhydrous sodium sulfate, and the solvent was evaporated away to give a yellow reaction mixture. The intended product was roughly purified through column chromatography (hexane/ethyl acetate=3/1), dissolved in diethyl ether, then methanol was added thereto, and the resultant precipitate was filtered to give a yellow powder of an intermediate 6 in a yield amount of 350 mg (0.39 mmol) and a yield of 78%.

¹H-NMR (500 MHz, toluene d-8): δ=7.47 (s, 2H, ArH_{f}), 7.27 (s, 4H, ArH_{d,e}), 7.16 (d, 8H, J=8.0 Hz, ArH_{c}), 6.75 (d, 8H, J=9.0 Hz, ArH_{b}), 3.36 (s, 12H, ArHₐ), 1.44 (s, 6H, ArHₕ), 1.38 (s, 12H, ArH_{g})

### MS (ASAP) m/z 897.31 [M⁺].

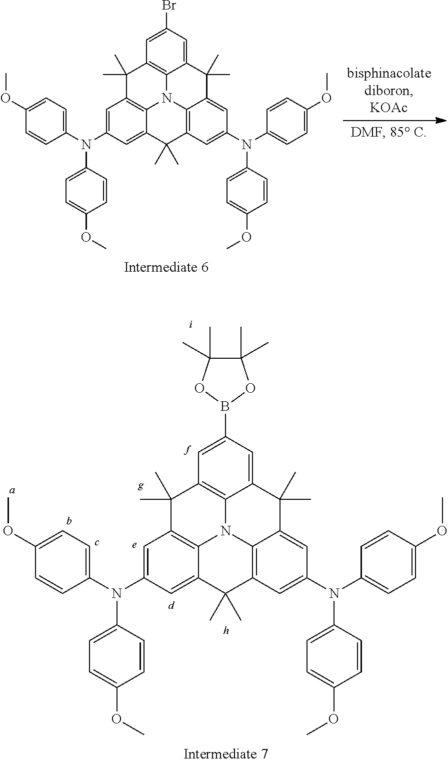 [SMILES: COc1ccc(N(c2ccc(OC)cc2)c2cc3c4c(c2)C(C)(C)c2cc(N(c5ccc(OC)cc5)c5ccc(OC)cc5)cc5c2N4c2c(cc(Br)cc2C5(C)C)C3(C)C)cc1 ; COc1ccc(N(c2ccc(OC)cc2)c2cc3c4c(c2)C(C)(C)c2cc(N(c5ccc(OC)cc5)c5ccc(OC)cc5)cc5c2N4c2c(cc(B4OC(C)(C)C(C)(C)O4)cc2C5(C)C)C3(C)C)cc1]

In a nitrogen stream, the intermediate 6 (160 mg, 0.17 mmol), bispinacolate &boron (51 mg, 0.2 mmol), and potassium acetate (59 mg, 0.6 mmol) were dissolved in dimethylformamide (20 mL), Pd(dppf)Cl₂.CH₂Cl₂ (4.1 mg, 0.005 mmol) was added thereto, and stirred at 85° C. for 15 hours. After the reaction, the solvent was evaporated away, and the residue was processed for liquid-liquid separation (dichloromethane), dried with anhydrous sodium sulfate, roughly purified through column chromatography (hexane/ethyl acetate=4/1), dissolved in diethyl ether, methanol was added thereto, and the resultant precipitate was taken out through filtration to give a yellow powder of an intermediate 7 in a yield amount of 92.0 mg (0.097 mmol) and a yield of 57%.

¹H-NMR (500 MHz, toluene d-8): δ=8.20 (s, 2H, ArH_{f}), 7.34 (d, 2H, J=2.5 Hz, ArH_{dore}), 7.26 (d, 2H, J=2.5 Hz, ArH_{dore}), 7.16 (d, 8H, J=9.0 Hz, ArH_{c}), 6.73 (d, 8H, J=9.0 Hz, ArH_{b}), 3.36 (s, 12H, ArHₐ), 1.57 (s, 12H, ArH_{g}), 1.45 (s, 6H, ArHₕ), 1.20 (s, 12H, ArHᵢ)

### MS (ASAP) m/z 945.34 [M⁺].

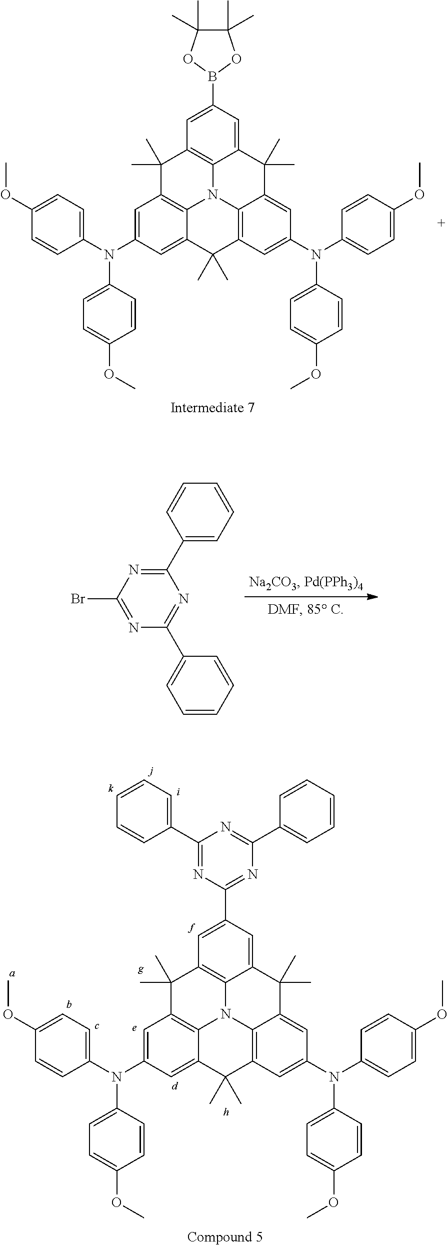 [SMILES: COc1ccc(N(c2ccc(OC)cc2)c2cc3c4c(c2)C(C)(C)c2cc(N(c5ccc(OC)cc5)c5ccc(OC)cc5)cc5c2N4c2c(cc(B4OC(C)(C)C(C)(C)O4)cc2C5(C)C)C3(C)C)cc1 ; Brc1nc(-c2ccccc2)nc(-c2ccccc2)n1 ; COc1ccc(N(c2ccc(OC)cc2)c2cc3c4c(c2)C(C)(C)c2cc(N(c5ccc(OC)cc5)c5ccc(OC)cc5)cc5c2N4c2c(cc(-c4nc(-c6ccccc6)nc(-c6ccccc6)n4)cc2C5(C)C)C3(C)C)cc1]

In a nitrogen stream, the intermediate 7 (92 mg, 0.097 mmol), and 2-bromo-4,6-diphenyl-1,3,5-triazine (118 mg, 0.38 mmol) were dissolved in toluene (20 mL), 2 mol/L, sodium carbonate (2 mL) and Pd(PPh₃)₄ (50 mg, 0.043 mmol) were added. thereto, and heated under reflux for 36 hours. After the reaction, the mixture was processed for liquid-liquid separation, and purified through column chromatography (hexane/ethyl acetate=4/1) to give a yellow powder of a compound 5 in a yield amount of 47 mg (0.045 mmol) and a yield of 46%.

¹H-NMR (500 MHz, toluene d-8): δ=9.06 (s, 2H, ArHₖ), 8.98 (dd, 4H, J=6.0 Hz, 2.0 Hz, ArHⱼ), 7.42, 7.04, 7.36, 7.31 (m, 10H, ArH_{d,e,f,i}), 7.21 (dd, 8H, J=7.0 Hz, 2.0 Hz, ArH_{c}), 6.78 (d, 8H, J=7.0 Hz, 2.0 Hz, ArH_{b}), 3.39 (s, 12H, ArHₐ), 1.69 (s, 12H, ArH_{g}), 1.50 (s, 6H, ArHₕ)

### MS (ASAP) m/z 1050.99 [M⁺].

### (Synthesis Example 6) Synthesis of Compound 6

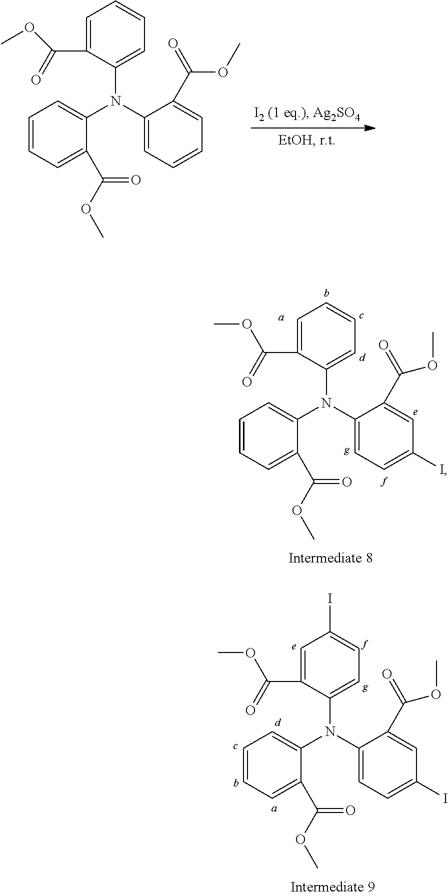

A mixture of trimethyl-2,2′,2″-nitrilotribenzoate (3.00 g, 7.16 mmol), and silver(I) sulfate (2.23 g, 7.16 mmol) was suspended in ethanol (230 mL), and iodine (1.82 g, 7.16 mmol) dissolved in ethanol (220 mL) was gradually and dropwise added thereto with cooling with ice. After dropwise addition, the reaction solution as stirred at room temperature for 12 hours. Solids were removed from the system through Celite filtration, and the solvent was removed with an evaporator. The precipitated solid was purified through silica gel column chromatography using dichloromethane as an eluent to give a pale orange powder of an intermediate 8 in a yield amount of 1.91 g and a yield of 49% and a pale orange powder of an intermediate 9 in a yield amount of 0.407 g and a yield of 8%.

### Intermediate 8:

¹H-NMR (500 MHz, CDCl₃): δ=7.87 (d, 1H, J=2.0 Hz, ArH_{c}), 7.62 (dd, 1H, J=8.0 Hz, 7.5 Hz, ArH_{f}), 7.60 (d, 1H, J=8.5 Hz, ArHₐ), 7.38 (dd, 1H, J=8.0 Hz, 7.5 Hz, ArH_{c}), 7.12 (ddd, 2H, J=7.5 Hz, 7.5 Hz, 3.5 Hz, ArH_{b}), 7.06 (dd, 2H, J=8.0 Hz, 2.0 Hz, ArH_{d}), 6.76 (d, 1H, J=8.5 Hz, ArH_{g}), 3.43 (s, 3H, CH₃), 3.41 (s, 3H, CH₃), 3.35 (s, 3H, CH₃), MS (ASAP) m/z 545.38 [M⁺].

### Intermediate 9:

¹H-NMR (500 MHz, CDCl₃): δ=7.89 (dd, 2H, J=5.5 Hz, 2.0 Hz, ArHₑ), 7.63 (d, 2H, J=7.0 Hz, ArH_{f}), 7.62 (d, 1H, J=9.0 Hz, ArHₐ), 7.39 (dd, 1H, J=8.5 Hz, 7.5 Hz, ArH_{c}), 7.15 (dd, 1H, J=7.5 Hz, 8.0 Hz, ArH_{b}), 7.06 (d, 2H, J 32 9.0 Hz, ArH_{d}), 6.77 (dd, 2H, J=8.5 Hz, 9.0 Hz, ArH_{g}), 3.45 (s, 3H, CH₃), 3.41 (s, 3H, CH₃), 3.38 (s, 3H, CH₃); MS (ASAP) m/z 671.14 [M⁺].

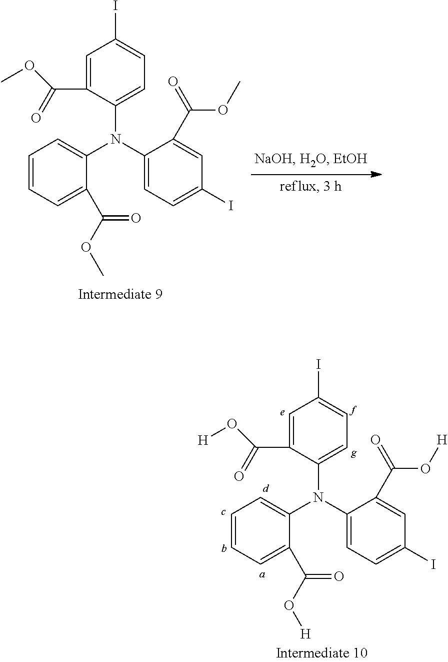

Sodium hydroxide (0.795 g, 19.9 mmol) was added to the intermediate 9 (1.067 g, 1.59 mmol), then heated under reflux in a mixed solution of ethanol/water=1/1 (6.7 mL) for 3 hours, and hydrochloric acid was dropwise added thereto to adjust the pH of the reaction solution to around 2. The precipitated solid was collected through filtration under reduced pressure, and then dried under reduced pressure to give a white powder of an intermediate 10 in a yield amount of 0.913 g and a yield of 91%. ¹H-NMR (500 MHz, DMSO-d6): δ=12.75 (broad, 3H, COOH), 7.93 (broad, 2H, ArH_{c}), 7.75 (broad, 3H, ArH_{a,f}), 7.47 (broad, 1H, ArH_{c}), 7.23 (dd, 1H, ArH_{b}), 6.85 (b, 1H, ArH_{d}), 6.62 (b, 2H, ArH_{g}); MS (ASAP) m/z 629.02 [M⁺], 585.03 [M⁺−CO₂].

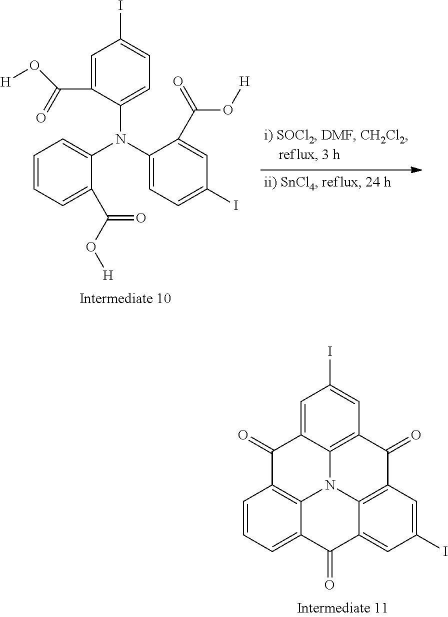 [SMILES: O=C(O)c1ccccc1N(c1ccc(I)cc1C(=O)O)c1ccc(I)cc1C(=O)O ; O=c1c2cccc3c(=O)c4cc(I)cc5c(=O)c6cc(I)cc1c6n(c23)c45]

Thionyl chloride (2.56 mL, 35.7 mmol) and dimethylformamide (0.30 mL) were added to the intermediate 10 (0.752 g, 1.20 mmol) in dewatered dichloromethane (45 mL), and heated under reflux for 3 hours in a nitrogen atmosphere. Subsequently, tin(IV) tetrachloride (2.50 mL, 21.4=mmol) was added thereto and further heated under reflux for 18 hours. After the reaction, an aqueous sodium hydroxide solution was gradually and dropwise added with stirring, and further stirred at room temperature for 1 hour. The resultant solid was collected through suction filtration, and dried under reduced pressure. Subsequently, the solid was recrystallized with nitrobenzene to give a yellow solid of an intermediate 11 in a yield amount of 0.336 g and a yield of 49%.

### MS (ASAP) m/z 575.09 [M⁺].

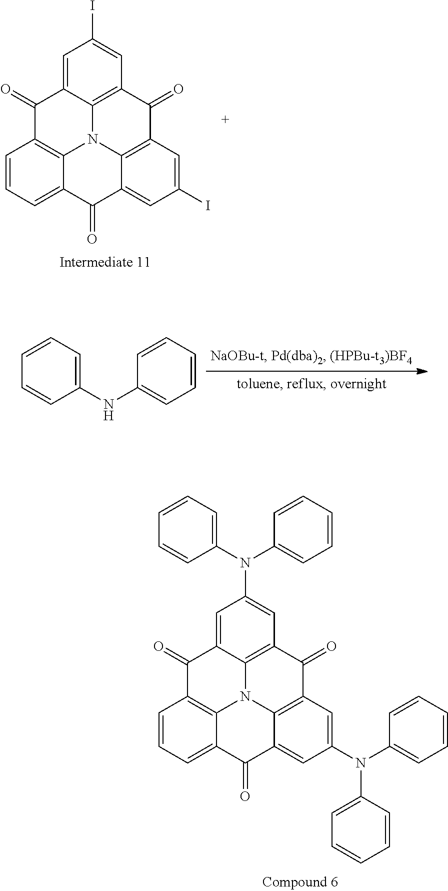 [SMILES: O=c1c2cccc3c(=O)c4cc(I)cc5c(=O)c6cc(I)cc1c6n(c23)c45 ; c1ccc(Nc2ccccc2)cc1 ; O=c1c2cccc3c(=O)c4cc(N(c5ccccc5)c5ccccc5)cc5c(=O)c6cc(N(c7ccccc7)c7ccccc7)cc1c6n(c23)c45]

Dewatered toluene (12 mL) was added to a mixture of the intermediate 11 (0.300 g, 0.521 mmol), diphenylamine (1.13 g, 10.4 mmol), sodium tert-butoxide (2.00 g, 20.8 mmol), bisdibenzylidene acetone palladium (0.0300 g, 0.0521 mmol), and tri-tert-butylphosphonium tetrafluoronorate (0.0151 g, 0.0521 mmol), then degassed and thereafter heated under reflux for some time in a nitrogen atmosphere. After the reaction, the reaction mixture was processed for liquid-liquid separation, then extracted with dichloromethane, and the organic layer was dried with sodium sulfate. Sodium sulfate was removed by filtration, and the solvent was removed with an evaporator. The precipitated solid was roughly purified through silica gel column chromatography using dichloromethane as an eluent. The resultant solid was purified through sublimation to give a dark purple solid of a compound 6 in a yield amount of 0.0864 g and a yield of 25%.

¹H-NMR (500 MHz, CDCl₃): δ=8.96 (dd, 2H, J=7.5 Hz, ArH_{b}), 8.96 (dd, 2H, J=7.5 Hz, ArH_{b}), (dd, 1H, J=7.5 Hz, ArHₐ), 7.34 (dd, 8H, J=7.5 Hz, 8.0 Hz, ArH_{e,g}), 7.19 (d, 8H, J=8.0 Hz, ArH_{d,h}), 7.15 (dd, 4H, J=7.5 Hz, 7.5 Hz, ArH_{f}), MS (ASAP) m/z 657.21 [M⁺].

### (Synthesis Example 7) Synthesis of Compound 7

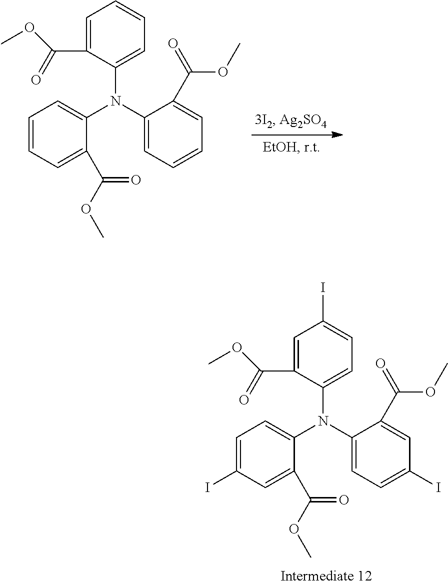

A mixture of trimethyl-2,2′,2″-nitrilotribenzoate (3.01 g, 7.16 mmol), and silver(i) sulfate (5.40 g, 21.5 mmol) was suspended in ethanol (150 mL), and iodine (6.69 g, 21.5 mmol) dissolved in ethanol (300 mL) was gradually and dropwise added thereto with cooling with ice. After the addition, the reaction solution was stirred at room temperature for 15.5 hours. Solids were removed from the system through Celite filtration, and the solvent was removed with an evaporator. The precipitated solid was purified through silica gel column chromatography using dichloromethane as an eluent to give a beige powdery solid of an intermediate 12 in a yield amount of 1.16 g and a yield of 20%.

### MS (ASAP) m/z 545.38 [M⁺].

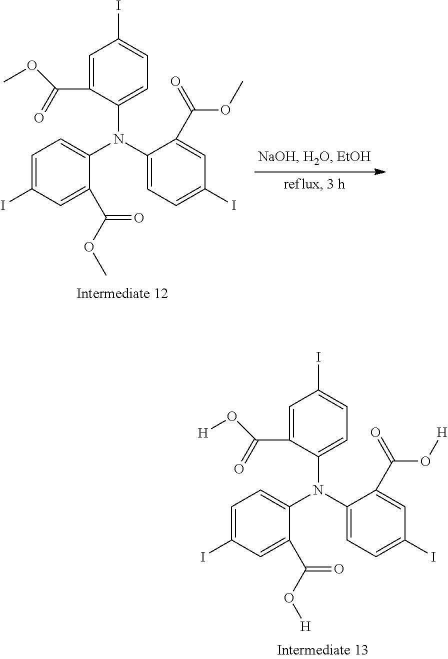 [SMILES: COC(=O)c1cc(I)ccc1N(c1ccc(I)cc1C(=O)OC)c1ccc(I)cc1C(=O)OC ; O=C(O)c1cc(I)ccc1N(c1ccc(I)cc1C(=O)O)c1ccc(I)cc1C(=O)O]

Sodium hydroxide (0.795 g, 19.9 mmol) was added to the intermediate 12 (1.27 g, 1.59 mmol), then heated under reflux in a mixed solvent of ethanol/water=1/1 (6.7 mL) for 3 hours, and hydrochloric acid was dropwise added thereto to adjust the pH of the reaction solution to about 2. The precipitated solid was collected through filtration under reduced pressure, and dried under reduced pressure to give a white powder of an intermediate 13 in a yield amount of 1.18 g and a yield of 98%.

MS (ASAP) m/z 755.00 [M⁺], 710.99 [M⁺-CO₂].

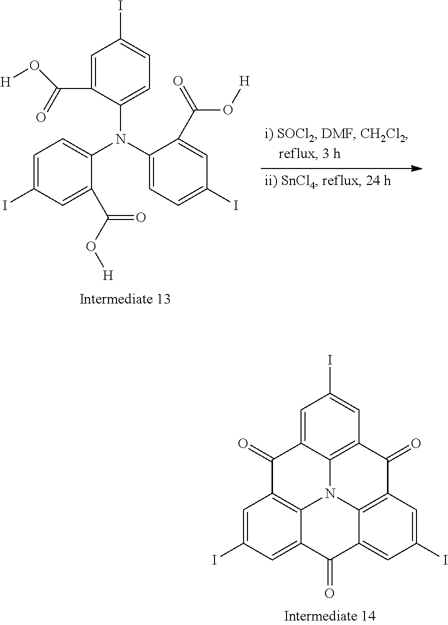 [SMILES: O=C(O)c1cc(I)ccc1N(c1ccc(I)cc1C(=O)O)c1ccc(I)cc1C(=O)O ; O=c1c2cc(I)cc3c(=O)c4cc(I)cc5c(=O)c6cc(I)cc1c6n(c23)c45]

Thionyl chloride (2.56 mL, 35.7 mmol) and dimethylformamide (0.30 mL) were added to the intermediate 13 (0.900 g, 1.19 mmol) in dewatered dichloromethane (50 mL), and heated under reflux for 3 hours in a nitrogen atmosphere. Subsequently, tin(IV) tetrachloride (2.50 mL, 21.4 mmol) was added, and further heated under reflux for 18 hours. After the reaction, an aqueous sodium hydroxide solution was gradually and dropwise added with stirring, and further stirred at room temperature for 1 hour. The resultant solid was collected through suction filtration, and dried under reduced pressure, Subsequently, the solid was recrystallized with nitrobenzene to give a yellow solid of an intermediate 14 in a yield amount of 0.553 g and a yield of 66%. MS (ASAP) m/z 700.99 [M⁺].

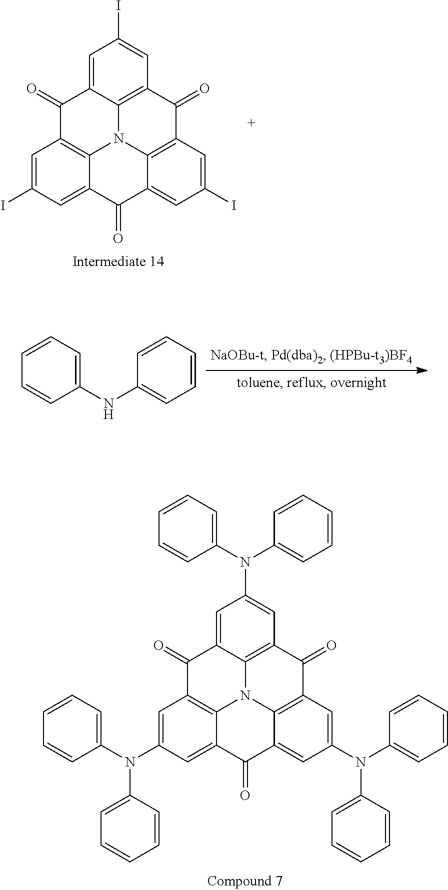 [SMILES: O=c1c2cc(I)cc3c(=O)c4cc(I)cc5c(=O)c6cc(I)cc1c6n(c23)c45 ; c1ccc(Nc2ccccc2)cc1 ; O=c1c2cc(N(c3ccccc3)c3ccccc3)cc3c(=O)c4cc(N(c5ccccc5)c5ccccc5)cc5c(=O)c6cc(N(c7ccccc7)c7ccccc7)cc1c6n(c23)c45]

Dewatered toluene (15 mL) was added to a mixture of the intermediate 14 (0.450 g, 0.642 mmol), diphenylamine (3,25 g, 19.3 mmol), sodium tort-butoxide (3.70 g, 38.5 mmol, bisdibenzylidene acetone palladium (0.0367 g, 0.0642 mmol), and tri-tert-butylphosphonium tetrafluoroborate (0.0186 g, 0.0642 mmol) and degassed, and then heated under flux for 19 hours in a nitrogen atmosphere. After the reaction, the reaction mixture was processed for liquid-liquid separation, extracted with dichloromethane, and the organic layer was dried with sodium sulfate. Sodium sulfate was removed through filtration, and the solvent was removed with an evaporator. The precipitated solid was roughly purified through silica gel column chromatography using dichloromethane as an eluent. The resultant solid was purified through sublimation to give a dark purple solid of a compound 7 in a yield amount of 0.0910 g and a yield of 17%.

¹H-NMR (500 MHz, CDCl₃): δ=8.57 (s, 6H ArH_{f}), 7.32 (dd, 12H, J=8.0 Hz, 7.5 Hz, ArH_{b,d}), 7.17 (d, 12H, J=7.5 Hz, ArH_{a,c}), 7.13 (dd, 12H, J=7.5 Hz, 7.5 Hz, ArH_{c}), MS (ASAP) m/z 825.16 [M⁺].

### (Example 1) Production Evaluation of Organic Photoluminescent Device Using Compound 1

In an Ar atmosphere glove box, a toluene solution of the compound 1 (concentration 1.0×10⁻⁵ mol/L) was prepared.

According to a spin coating method, a thin film (polymer film) composed of the compound 1 and polymethyl methacrylate was formed on a quartz substrate in a thickness of 200 nm to be an organic photoluminescent device. At this time, the concentration of the compound I was 0.1 mol % or 10 mol %. In the polymer film in which the concentration of the compound 1 was 0.1 mol %, the compound 1 existed as uniformly dispersed therein, while in the polymer film in which the concentration of the compound 1 was 10 mol %, the compound 1 became massed together.

In addition, according to a vacuum evaporation method, a thin film of the compound 1 (single film) was formed on a quartz substrate under a condition of a vacuum degree of 4×10⁻⁴ Pa or less in a thickness of 100 nm to be an organic photoluminescent device.

Apart from these and according to a vacuum evaporation method, the compound 1 and DPEPO were vapor-deposited from different evaporation sources on a quartz substrate under a condition of a vacuum degree of 4×10⁻⁴ Pa or less, thereby forming a thin film (doped film) having a concentration of the compound 1 of 0.5% by weight, 2% by weight or 10% by weight in a thickness of 40 nm to be an organic photoluminescent device.

In addition, a doped film containing the compound 1 was formed in the same mariner as above except that mCP or mCBP was sued in place of DPEPO to be an organic photoluminescent device.

In a state uniformly dissolved in toluene (homogeneous system), the compound 1 was electrochemically measured, and the HOMO level thereof was −5.16 eV and the LUMO level thereof was −2.13 eV. The HOMO level of the deposited single film (aggregated system) of the compound 1, determined through photoelectron spectroscopy in air and from the absorption end of the absorption spectrum thereof, was −5.51 eV, and the LUMO level thereof was −2.81 eV. From the fluorescent spectrum and the phosphorescent spectrum of the toluene solution of the compound 1, the excited singlet energy level E_{S1} of the compound 1 was estimated to be 2.884 eV, the excited triplet energy level E_{T1} thereof was to be 2.758 eV, and the difference ΔE_{ST} between the excited singlet energy level and the excited triplet energy level was to be 0.128 eV.

The toluene solution, the polymer film, the single film and the doped film with the compound 1 produced in Example 1 were evaluated for the emission characteristics thereof Data of the photoluminescent quantum yield (PL quantum yield) measured in air or in an argon atmosphere are shown in Table 1. The emission maximum wavelength of the toluene solution of the compound I was 460 nm, and the emission maximum wavelength of the single film of the compound 1 was 490 nm.

TABLE 1

| | | | | PL Quantum Yield | | Excitation |
|---|---|---|---|---|---|---|
| | | Concentration | | | in argon | Light Energy |
| Type of Device | Medium | of Compound 1 | | in air | atmosphere | (nm) |
| Solution of compound 1 | toluene | 1 × 10⁻⁵ | mol/L | 0.644 | 0.835 | 415 |
| Polymer film containing | PMMA | 0.1 | mol % | 0.245 | 0.257 | 415 |
| compound 1 | PMMA | 10 | mol % | 0.143 | 0.157 | 415 |
| Doped film with | DPEPO | 0.5 | wt % | 0.437 | 0.497 | 415 |
| compound 1 | DPEPO | 2 | wt % | 0.584 | 0.633 | 415 |
| | DPEPO | 10 | wt % | 0.155 | 0.253 | 415 |
| | mCP | 2 | wt % | 0.364 | 0.433 | 415 |
| | mCP | 10 | wt % | 0.530 | 0.614 | 415 |
| | mCBP | 2 | wt % | 0.718 | 0.848 | 340 |
| | mCBP | 10 | wt % | 0.758 | 0.847 | 340 |
| Single film of | — | 100 | wt % | 0.21 | 0.22 | 415 |
| compound 1 | | | | | | |

As shown in Table 1, the toluene solution, the polymer film and the doped film with the compound 1 all showed a higher PL quantum yield in an argon atmosphere than in air. This is considered to be because, in an argon atmosphere, deactivation of the triplet exciton by oxygen could be suppressed. This suggests that the phosphorescent emission process of the compound 1 may include a reverse intersystem crossing step from the excited triplet state T₁ to the excited single state S₁.

In addition, the polymer film, the single film and the doped film with the compound 1 were analyzed to measure the transient decay curve of emission thereof a 300 K, and it was known that all the films gave delayed fluorescence. Further, as a result of measurement in a range of 300 to 10 K, temperature dependency to increase the emission lifetime with the rise of temperature was recognized.

From these results, it is known that the compound 1 is a thermal activation type delayed fluorescent material that emits light via reverse intersystem crossing from the excited triplet state T₁ to the excited single state S₁ thereof.

### (Examples 2 to 4) Production and Evaluation of Organic Photoluminescent Devices using Compounds 2 to 4

A toluene solution, a polymer film and a doped film with any of the compounds 2 to 4 were produced to be organic photoluminescent devices in the same manner as in Example 1, except that any of the compounds 2 to 4, 6 and 7 were used in place of the compound 1 and mCBP only was used as the host material for the doped film. However, the concentration of the compounds 2 to 4, 6 and 7 was 1.0×10⁻⁵ mol/L in the toluene solution, 0.1 mol % in the polymer film, and 3% by weight in the doped film.

### (Example 5) Production and Evaluation of Organic Photoluminescent Device using Compound 5

In an Ar atmosphere glove box, a toluene solution of the compound 5 (concentration 1.0×10⁻⁵ mol/L) and a cyclohexane solution thereof (concentration 1.0×10⁻⁵mol/L) were prepared to be organic photoluminescent devices.

### (Comparative Example 1) Preparation and Evaluation of Toluene Solution of Comparative Compound 1

In an Ar atmosphere glove box, a toluene solution of a comparative compound 1 (concentration 1.0×10⁻⁵ mol/L) was prepared to a comparative sample.

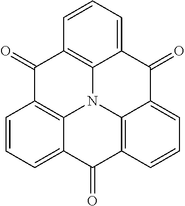 [SMILES: O=c1c2cccc3c(=O)c4cccc5c(=O)c6cccc1c6n(c23)c45]

The HOMO level and the LUMO level, as measured in toluene, of the compounds 2 to 4, 6, 7 and the comparative compound 1 are shown in Table 2; and the excited singlet energy level E_{S1}, the excited triplet energy level E_{T1} and the energy difference ΔE_{ST} of the toluene solutions, the polymer films and the doped films produced in Examples 2 to 4, 6, 7 and Comparative Example 1 are shown in Table 3.

TABLE 2

| | HOMO Level | LUMO Level | Energy Gap |
|---|---|---|---|
| Compound No. | (eV) | (eV) | (eV) |
| Comparative | −6.9 | −2.9 | 4.0 |
| Compound 1 | | | |
| Compound 2 | −5.9 | −3.0 | 2.9 |
| Compound 3 | −5.3 | −2.9 | 2.4 |
| Compound 4 | −5.6 | −2.9 | 2.7 |
| Compound 6 | −5.5 | −2.8 | 2.7 |

TABLE 3

| | | | E_{S1} | E_{T1} | ΔE_{ST} |
|---|---|---|---|---|---|
| Example No. | Type of Device | Medium | (eV) | (eV) | (eV) |
| Comparative Example 1 | Solution of comparative compound 1 | toluene | 3.05 | 2.68 | 0.37 |
| Example 2 | Solution of compound 2 | toluene | 2.64 | 2.41 | 0.23 |
| | Polymer film containing compound 2 | PMMA | 2.77 | 2.51 | 0.27 |
| | Doped film with compound 2 | mCBP | — | — | 0.061 |
| Example 3 | Solution of compound 3 | toluene | 2.48 | 2.32 | 0.16 |
| | Polymer film containing compound 3 | PMMA | 2.68 | 2.51 | 0.17 |
| | Doped film with compound 3 | mCBP | — | — | 0.036 |
| Example 4 | Solution of compound 4 | toluene | 2.41 | 2.07 | 0.34 |
| | Polymer film containing compound 4 | PMMA | 2.49 | 2.30 | 0.19 |
| | Doped film with compound 4 | mCBP | — | — | 0.114 |
| Example 6 | Solution of compound 6 | toluene | 2.33 | 2.06 | 0.27 |
| | Polymer film containing compound 6 | PMMA | 2.38 | 2.07 | 0.31 |
| Example 7 | Solution of compound 7 | toluene | 2.28 | 1.98 | 0.30 |
| | Polymer film containing compound 7 | PMMA | 2.28 | 1.95 | 0.33 |

The toluene solutions, the polymer films and the doped films produced in Examples 2 to 7 and. Comparative Example 1, and the cyclohexane solution produced in Example 5 were evaluated for emission characteristics. The PL, quantum yield and the emission lifetime of each toluene solution and the cyclohexane solution, as analyzed in air or in the absence of oxygen, are shown in Table 4; and the PL quantum yield of each polymer film and each doped film, as measured in air or in the absence of oxygen, and the emission lifetime thereof, as measured in the absence of oxygen, are shown in. Table 5. Here, in the absence of oxygen means after nitrogen bubbling for the toluene solution and the cyclohexane solution, and means in an argon atmosphere for the polymer film and the doped film.

TABLE 4

| | | | | | | Emission Lifetime | | | | Excitation Light | |
|---|---|---|---|---|---|---|---|---|---|---|---|
| | | | | | | Instantaneous | | Delayed | | Wavelength (nm) | |
| | | | Emission | PL Quantum Yield | | Fluorescence (ns) | | Fluorescence (μs) | | at the time of | at the time |
| | | | Maximum | in the | in the | in the | in the | in the | in the | measurement | of |
| | | | Wave- | presence | absence | presence | absence | presence | absence | of PL | measurement |
| Example | Type of | | length | of | of | of | of | of | of | quantum | of emission |
| No. | Device | Medium | (nm) | oxygen | oxygen | oxygen | oxygen | oxygen | oxygen | yield | lifetime |
| Comparative | Comparative | toluene | 425 | <0.01 | <0.01 | 1.2 | 1.3 | 0.6 | 0.7 | 310 | 405 |
| Example 1 | Compound 1 | | | | | | | | | | |
| Example 2 | Compound 2 | toluene | 515 | 0.111 | 0.168 | 9.0 | 10.2 | 0.2 | 3.6 | 410 | 405 |
| Example 3 | Compound 3 | toluene | 550 | 0.057 | 0.140 | 8.1 | 8.7 | 0.3 | 30.9 | 360 | 365 |
| Example 4 | Compound 4 | toluene | 580 | 0.135 | 0.193 | 12.8 | 17.6 | 0.1 | 43.9 | 470 | 405 |
| Example 5 | Compound 5 | cyclo- | 520 | 0.74 | 0.86 | 5.1 | 5.8 | — | — | 470 | 365 |
| | | hexane | | | | | | | | | |
| | | toluene | 580 | 0.53 | 0.63 | 5.3 | 6.0 | — | 25.9 | 470 | 365 |
| Example 6 | Compound 7 | toluene | 580 | 0.11 | 0.13 | 6.4 | 6.9 | — | 151.0 | 510 | 340 |
| Example 7 | Compound 8 | toluene | 580 | 0.06 | 0.07 | 5.1 | 5.5 | — | 103.5 | 550 | 340 |

TABLE 5

| | | | | | Emission Lifetime | |
|---|---|---|---|---|---|---|
| | | | | | Instantaneous | Delayed |
| | | | PL Quantum Yield | | Fluorescence | Fluorescence |
| | | | in the | in the | (ns) | (μs) |
| | | | presence | absence | in the absence | in the absence |
| Example No. | Type of Device | Medium | of oxygen | of oxygen | oxygen | of oxygen |
| Example 2 | Polymer film | PMMA | 0.345 | 0.632 | 3.6 | 19.2 |
| | containing compound 2 | | | | | |
| | Doped film with | mCBP | 0.606 | 0.732 | 7.5 | 771 |
| | compound 2 | | | | | |
| Example 3 | Polymer film | PMMA | 0.168 | 0.474 | 4.9 | 46.5 |
| | containing compound 3 | | | | | |
| | Doped film with | mCBP | 0.514 | 0.659 | 8.6 | 267 |
| | compound 3 | | | | | |
| Example 4 | Polymer film | PMMA | 0.223 | 0.431 | 8.6 | 4.2 |
| | containing compound 4 | | | | | |
| | Doped film with | mCBP | 0.286 | 0.503 | 13 | 5810 |
| | compound 4 | | | | | |
| Example 5 | Polymer film | PMMA | 0.223 | 0.431 | 5.89 | 79.4 |
| | containing compound 5 | | | | | |
| Example 6 | Polymer film | PMMA | 0.115 | 0.117 | 5.1 | 4.0 |
| | containing compound 6 | | | | | |
| Example 7 | Polymer film | PMMA | 0.075 | 0.091 | 5.4 | 163 |
| | containing compound 7 | | | | | |

As shown in Tables 4 and 5, the solutions containing any of the compounds 2 to 5, and the polymer films and the doped films with any of the compounds 2 to 4 all exhibited a higher PL quantum yield in the absence of oxygen than in air, in which a delayed fluorescent component on a microsecond order was observed. From the data of the delayed fluorescence lifetime of the toluene solutions show in Table 4, it is known that removing oxygen tends to increase the delayed fluorescence lifetime. These results can be considered to be because the deactivation of the triplet exciton by oxygen is suppressed, and also for the compounds 2 to 5, intervention of reverse intersystem crossing from the excited triplet state T₁ to the excited single state S₁ may be in the fluorescence emission process thereof The polymer films and the doped films with any of the compounds 2 to 5 were analyzed to draw the transient decay curve of emission thereof at 300 to 10 K, in which temperature dependency to increase the emission lifetime with the rise of temperature was recognized. From these results, it is known that the compounds 2 to 5 are also thermal activation type delayed fluorescent materials that emit light via reverse intersystem crossing from the excited triplet state T₁ to the excited single state S₁ thereof.

### (Example 6) Production of Organic Electroluminescent Device using Compound 1 (0.5 wt %) and DPEPO in Light-Emitting Layer

On a glass substrate with an anode of indium tin oxide (ITO) having a thickness of 100 nm framed thereon, thin films were layered according to a vacuum vapor deposition method under a vacuum degree of 4.0×10⁻⁴ Pa. First, on ITO, NPD was formed in a thickness of 30 nm, and then TCTA was formed thereon in a thickness of 20 nm. Next, the compound 1 and DPEPO were co-deposited from different evaporation sources to form a layer having a thickness of 40 nm as a light-emitting layer. At this time, the concentration of the compound 1 was 0.5% by weight. Next, DPEPO was formed in a thickness of 10 nm, and TPBi was formed thereon in a thickness of 30 nm. Further, lithium fluoride (LiF) was formed in a thickness of 0.8 nm, and then aluminum (Al) was vapor-deposited thereon in a thickness of 80 nm to form a cathode, thereby producing an organic electroluminescent device.

### (Examples 7, 8) Production of Organic Electroluminescent Device using Compound 1 (2 wt % or 10 wt %) and DPEPO in Light-Emitting Layer

Organic electroluminescent devices were produced in the same manner as in Example 6, except that the concentration of the compound 1 in the light-emitting layer was changed to 2% by weight or 10% by weight.

### (Example 9) Production of Organic Electroluminescent Device having two Light-Emitting Layers each having a Different Concentration of the Compound 1

An organic electroluminescent device was produced in the same manner as in Example 6, except that, in place of forming a light-emitting layer having a concentration of the compound 1 of 0.5% by weight, a light-emitting layer having a concentration of the compound 1 of 10% by weight was formed in a thickness of 20 nm and a light-emitting layer having a concentration of the compound 1 of 2% by weight was formed thereon in a thickness of 20 nm to thereby forming a 2-layered light emitting layer.

### (Examples 10, 11) Production of organic electroluminescent device using compound 1 (2 wt % or 10 wt %) and mCP in light-emitting layer

Organic electroluminescent devices were produced in the same manner as in Example 6, except that mCP was used in place of DPEPO and the concentration of the compound 1 in the light-emitting layer was changed to 2% by weight or 10% by weight.

### (Example 12) Production of organic electroluminescent device using compound 1 (2 wt %) and mCBP in light-emitting layer

On a glass substrate with an anode of indium tin oxide (ITO) having a thickness of 100 nm formed thereon, thin films were layered according to a vacuum vapor deposition method under the same vacuum degree as in Example 1, First, on ITO, HAT-CN was formed in a thickness of 10 nm, and Tris-PCz was formed thereon in a thickness of 30 nm. Next, the compound 1 and mCBP were co-deposited from different evaporation sources to form a layer having a thickness of 30 nm as a light-emitting layer. At this time, the concentration of the compound 1 was 2% by weight, Next, T2T was formed in a thickness of 10 nm, and BPy-TP2 was formed thereon in a thickness of 40 nm. Further, lithium fluoride (LiF) was vapor-deposited in a thickness of 0.8 nm, and then aluminum (Al) was vapor-deposited thereon in a thickness of 80 nm to form a cathode, thereby producing an organic electroluminescent device.

### (Example 13) Production of Organic Electroluminescent Device using Compound 1 (10 wt %) and mCBP in Light-Emitting Layer

An organic electroluminescent device was produced in the same manner as in Example 12, except that the concentration of the compound 1 in the light-emitting layer was changed to 10% by weight.

### (Examples 14 to 16) Production and Evaluation of Organic Electroluminescent Devices using Compounds 2 to 4

Organic electroluminescent devices were produced in the same manner as in Example 12, except that the compound 2 was used in place of the compound 1 and the concentration of the compound 2 in the light-emitting layer was changed to 3% by weight.

Regarding the organic electroluminescent devices produced in Examples 6 to 12, the maximum external quantum efficiency obtained from the external quantum efficiency-current density characteristic thereof are shown in Table 6. In the column of device configuration in Table 6, the oblique line indicates a boundary between the adjacent layers, and the numerical value with a unit nm as parenthesized indicates a thickness of the layer.

TABLE 6

| | | Maximum |
|---|---|---|
| | | External |
| | | Quantum |
| | | Efficiency |
| Example No. | Device Configuration | (%) |
| Example 6 | ITO/NPD(30 nm)/TCTA(20 nm)/compound 1(0.5 wt %):DPEPO(40 nm)/DPEPO(10 nm)/TPBi(30 nm)/LiF/Al | 1.68 |
| Example 7 | ITO/NPD(30 nm)/TCTA(20 nm)/compound 1(2 wt %):DPEPO(40 nm)/DPEPO(10 nm)/TPBi(30 nm)/LiF/Al | 2.44 |
| Example 8 | ITO/NPD(30 nm)/TCTA(20 nm)/ compound 1(10 wt %):DPEPO(40 nm)/DPEPO(10 nm)/TPBi(30 nm)/LiF/Al | 6.58 |
| Example 9 | ITO/NPD(30 nm)/TCTA(20 nm)/ compound 1(10 w %):DPEPO(20 nm)/ compound 1(2 wt%):DPEPO | 10.23 |
| | (20 nm)/DPEPO(10 nm)/TPBi(30 nm)/LiF/Al | |
| Example 10 | ITO/NPD(30 nm)/TCTA(20 nm)/ compound 1(2 wt %):mCP(40 nm)/DPEPO(10 nm)/TPBi(30 nm)/LiF/Al | 0.86 |
| Example 11 | ITO/NPD(30 nm)/TCTA(20 nm)/ compound 1(10 wt %):mCP(40 nm)/DPEPO(10 nm)/TPBi(30 nm)/LiF/Al | 2.61 |
| Example 12 | ITO/HAT-CN(10 nm)/Tris-PCz(30 nm)/ compound 1(2 wt %):mCBP(30 nm)/T2T(10 nm)/Bpy-TP2(40 nm)/LiF/Al | 12.8 |
| Example 13 | ITO/HAT-CN(10 nm)Tris-PCz(30 nm)/ compound 1(10 wt %):mCBP(30m)/T2T(10 nm)/Bpy-TP2(40 nm)/LiF/Al | 16.8 |
| Example 14 | ITO/HAT-CN(10 nm)/Tris-PCz(30 nm)/ compound 2(3 wt %):mCBP(30 nm)/T2T(10 nm)/Bpy-TP2(40 nm)/LiF/Al | 14.1 |
| Example 15 | ITO/HAT-CN(10 nm)/Tris-PCz(30 nm)/ compound 3(3 wt %):mCBP(30 nm)/T2T(10 nm)/Bpy-TP2(40 nm)/LiFIAl | 16.3 |
| Example 16 | ITO/HAT-CN(10 nm)/Tris-PCz(30 nm)/ compound 4(3 wt %):mCBP(30 nm)/T2T(10 nm)/Bpy-TP2(40 nm)/LiF/Al | 14.4 |

As shown in Table 6, the compounds 1 to 4 realize organic electroluminescent devices having a high emission efficiency. The CIE chromaticity coordinate of the light emitted by the organic electroluminescent device using the compound 2 (Example 14) was (0.248, 0.611).

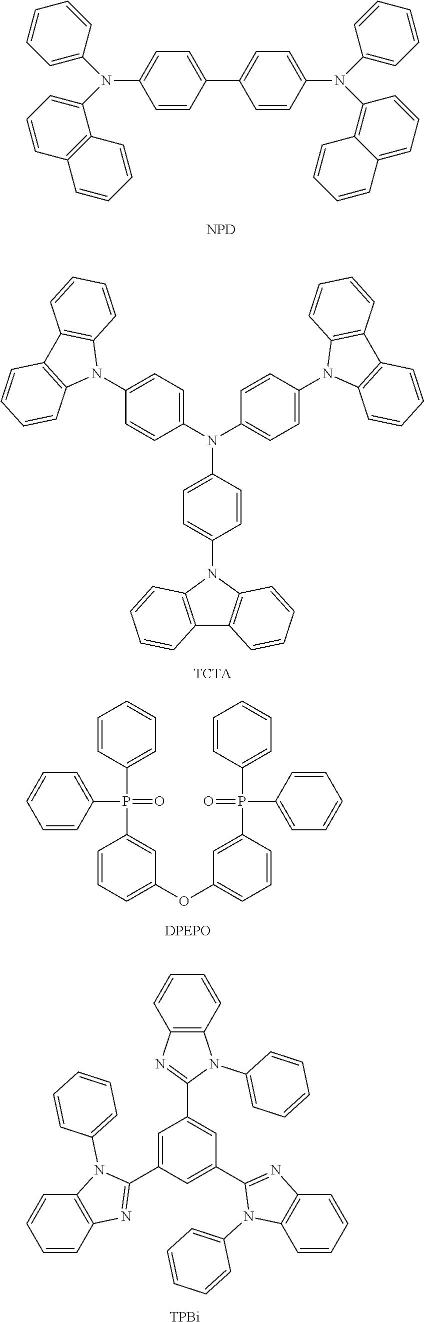

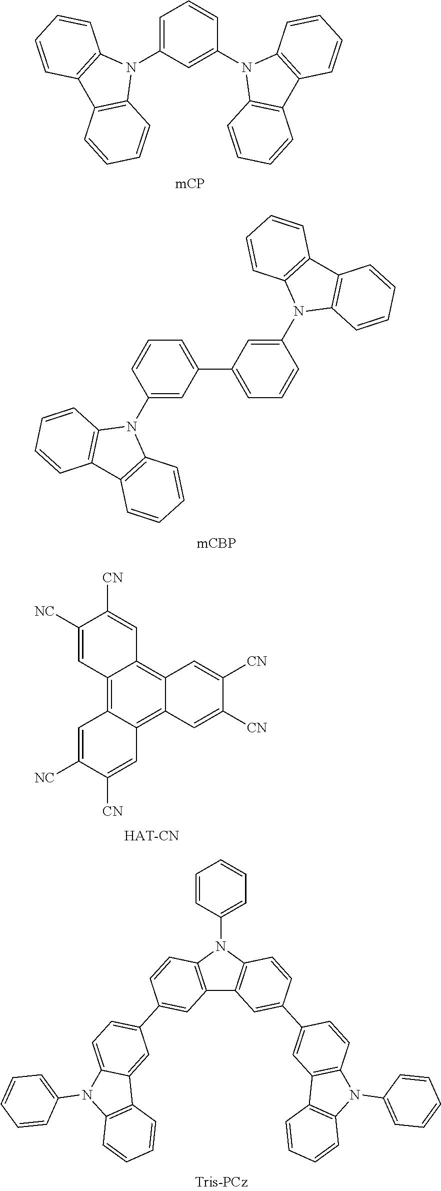 [SMILES: c1cc(-n2c3ccccc3c3ccccc32)cc(-n2c3ccccc3c3ccccc32)c1 ; c1cc(-c2cccc(-n3c4ccccc4c4ccccc43)c2)cc(-n2c3ccccc3c3ccccc32)c1 ; N#Cc1cc2c3cc(C#N)c(C#N)cc3c3cc(C#N)c(C#N)cc3c2cc1C#N ; c1ccc(-n2c3ccccc3c3cc(-c4ccc5c(c4)c4cc(-c6ccc7c(c6)c6ccccc6n7-c6ccccc6)ccc4n5-c4ccccc4)ccc32)cc1]

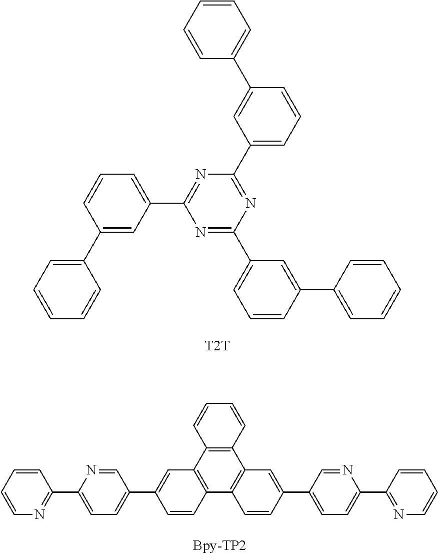 [SMILES: c1ccc(-c2cccc(-c3nc(-c4cccc(-c5ccccc5)c4)nc(-c4cccc(-c5ccccc5)c4)n3)c2)cc1 ; "T2T" ; c1ccc(-c2ccc(-c3ccc4c5ccc(-c6ccc(-c7ccccn7)nc6)cc5c5ccccc5c4c3)cn2)nc1]

### REFERENCE SIGNS LIST

1 Substrate
2 Anode
3 Hole Injection Layer
4 Hole Transport Layer
5 Light-Emitting Layer
6 Electron Transport Layer
7 Cathode

1-40. (canceled)

41. A compound having a structure represented by the following general formula (1): 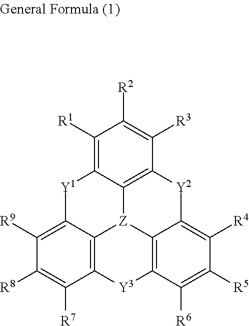
wherein R¹ to R⁹ each independently represent a hydrogen atom or a substituent, and at least one of R¹ to R⁹ is a substituent; Y¹ to Y³ each independently represent a substituted or unsubstituted methylene group (C(R¹⁰)(R¹¹) where R¹⁰ and R¹¹ each independently represent a hydrogen atom or a substituent), a carbonyl group (C═O), a thiocarbonyl group (C═S), a substituted or unsubstituted imino group (N(R¹²) where R¹² represents a hydrogen atom or a substituent), an oxygen atom, a sulfur atom, or a sulfonyl group (SO₂); Z represents a nitrogen atom, a boron atom, or a phosphine oxide group (P═O).

42. The compound according to claim 41, wherein in the general formula (1), one or two of R², R⁵ and R⁸ each are a substituted or unsubstituted diarylamino group having 12 to 40 carbon atoms, or a substituted or unsubstituted carbazolyl group having 12 to 40 carbon atoms, and at least one of Y¹ to Y³ is a carbonyl group (C═O).

43. The compound according to claim 41, wherein in the general formula (1), at least one of R¹ to R⁹ is a substituted or unsubstituted diarylamino group having 12 to 40 carbon atoms, and at least one of Y¹ to Y³ is a carbonyl group (C═O).

44. The compound according to claim 41, wherein in the general formula (1), at least one of R¹ to R⁹ is an aryl group having 6 to 40 carbon atoms and substituted with a substituted or unsubstituted diphenylamino group, and at least one of Y¹ to Y³ is a carbonyl group (C═O).

45. The compound according to claim 44, wherein the aryl group is a phenyl group.

46. The compound according to claim 41, represented by the following general formula (2): 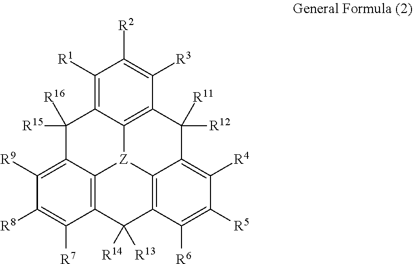
wherein R¹ to R⁹ each independently represent a hydrogen atom or a substituent, and at least one of R¹ to R⁹ is a substituted or unsubstituted heteroaryl group, provided that at least one ring skeleton-constituting atom adjacent to the atom that participates in bonding of the heteroaryl group is a nitrogen atom; R¹¹ to R¹⁶ each independently represent a substituent; Z represents a nitrogen atom, a boron atom, or a phosphine oxide group (P═O).

47. The compound according to claim 46, wherein the substituted or unsubstituted heteroaryl group is a substituted or unsubstituted diaryltriazinyl group, and R¹¹ to R¹⁶ in the general formula (2) each are independently a substituted or unsubstituted alkyl group.

48. The compound according to claim 46, wherein in the general formula (2), R² is a substituted or unsubstituted heteroaryl group, and at least one ring skeleton-constituting atom adjacent to the atom that participates in bonding of the group is a nitrogen atom; and at least one of R⁴ to R⁶, and R⁷ to R⁹ is a group containing a diarylamino structure, or a group containing a carbazole ring.

49. The compound according to claim 48, wherein the substituted or unsubstituted heteroaryl group is a substituted or unsubstituted triazinyl group.

50. The compound according to claim 48, wherein the group containing a diarylamino structure is a diarylamino group substituted with a substituent.

51. The compound according to claim 41, represented by the following general formula (3): 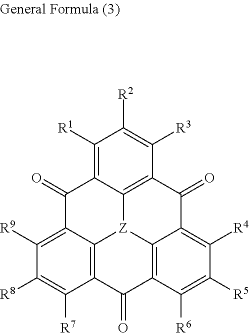
wherein R¹ to R⁹ each independently represent a hydrogen atom or a substituent, and at least one of R¹ to R⁹ is a substituent; Z represents a nitrogen atom, a boron atom or a phosphine oxide group (P═O).

52. The compound according to claim 51, wherein in the general formula (3), one or two of R², R⁵, and R⁸ each are a group containing a diarylamino structure or a group containing a carbazole ring.

53. The compound according to claim 51, wherein in the general formula (3), at least one of R¹ to R⁹ is a group containing a substituted or unsubstituted diarylamino group.

54. The compound according to claim 51, wherein in the general formula (3), at least one of R¹ to R⁹ is a group having a structure represented by the following general formula (4): 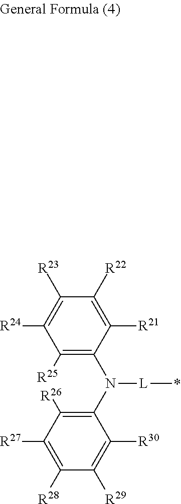
wherein R²¹ to R³⁰ each independently represent a hydrogen atom or a substituent; R²⁵ and R²⁶ may bond to each other to form a single bond or a linking group; L represents a single bond or a substituted or unsubstituted arylene group; * represents a bonding position.

55. The compound according to claim 54, wherein R²⁵ and R²⁶ in the general formula (4) do not bond to each other.

56. The compound according to claim 54, wherein L in the general formula (4) is a substituted or unsubstituted arylene group.

57. The compound according to claim 54, wherein L in the general formula (4) is a substituted or unsubstituted phenylene group.

58. The compound according to claim 54, wherein at least one of R²³ and R²⁴ in the general formula (4) is a hydrogen atom.

59. An organic light-emitting device containing a compound having a structure represented by the following general formula (1): 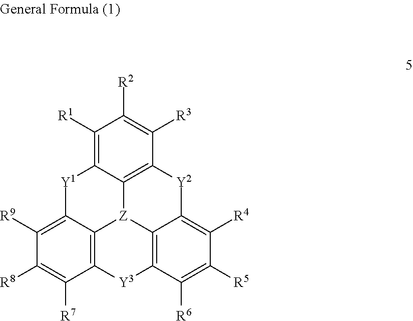
wherein R¹ to R⁹ each independently represent a hydrogen atom or a substituent, and at least one of R¹ to R⁹ is a substituent; Y¹ to Y³ each independently represent a substituted or unsubstituted methylene group (C(R¹⁰)(R¹¹) where R¹⁰ and R¹¹ each independently represent a hydrogen atom or a substituent), a carbonyl group (C═O), a thiocarbonyl group (C═S), a substituted or unsubstituted imino group (N(R¹²) where R¹² represents a hydrogen atom or a substituent), an oxygen atom, a sulfur atom, or a sulfonyl group (SO₂); Z represents a nitrogen atom, a boron atom, or a phosphine oxide group (P═O).

60. The organic light-emitting device according to claim 59, which emits delayed fluorescence.
